(19) United States
(12) Patent Application Publication
(10) Pub. No.: US 2017/0049513 A1
(43) Pub. Date: Feb. 23, 2017

(54) MULTIPLE ELECTRODE GENERATOR

(71) Applicants: Eric R. Cosman, JR., Belmont, MA (US); Eric R. Cosman, SR., Belmont, MA (US)

(72) Inventors: Eric R. Cosman, JR., Belmont, MA (US); Eric R. Cosman, SR., Belmont, MA (US)

(73) Assignee: COSMAN MEDICAL, INC., Burlington, MA (US)

(21) Appl. No.: 12/835,489

(22) Filed: Jul. 13, 2010

Related U.S. Application Data
(60) Provisional application No. 61/258,971, filed on Nov. 6, 2009.

(51) Int. Cl. A61B 18/18; A61B 18/14; A61B 18/12

(52) U.S. Cl. CPC A61B 18/18; A61B 18/14; A61B 2018/00791

(57) ABSTRACT

A system and a method for applying energy, particularly radiofrequency electrical energy, to a living body.

### CLAIM OF PRIORITY

This application claims the benefit of prior U.S. Provisional Application No. 61/258,971, filed on Nov. 6, 2009, which is incorporated by reference in its entirety.

### TECHNICAL FIELD

This invention relates generally to the advances in medical systems and procedures for prolonging and improving human life. The present invention relates generally to a system and method for applying energy, particularly radiofrequency electrical energy, to a living body. The present invention also relates generally to a system and method for apply energy for the purpose of tissue ablation, including the ablation of nervous tissue. The present invention also relates generally to a system and method for apply energy to a living body for the purpose of treating a medical disorder.

### BACKGROUND

The theory behind and practice of RF heat ablation has been known for decades, and a wide range of suitable RF generators and electrodes exists. For example, equipment for causing heat lesions is available from Radionics, Inc., located in Burlington, Mass. A research paper by E. R. Cosman, et al., entitled “Theoretical Aspects of Radio Frequency Lesions in the Dorsal Root Entry Zone,” Neurosurgery, Vol. 15, No. 6, pp. 945-0950 (1984), describes various techniques associated with radio frequency lesions and is hereby incorporated by reference herein in its entirety. Also, research papers by S. N. Goldberg, et al., entitled “Tissue Ablation with Radio Frequency: Effect of Probe Size, Gauge, Duration, and Temperature on Lesion Volume,” Acad. Radiol., Vol. 2, pp. 399-404 (1995), and “Thermal Ablation Therapy for Focal Malignancy,” AJR, Vol. 174, pp. 323-331 (1999), described techniques and considerations relating to tissue ablation with radio frequency energy and are hereby incorporated by reference herein in its entirety.

Examples of high frequency generators and electrodes are given in the papers of entitled “Theoretical Aspects of Radiofrequency Lesions and the Dorsal Root Entry Zone,” by Cosman, E. R., et al., Neurosurg 15:945-950, 1984; and “Methods of Making Nervous System Lesions,” by Cosman, E. R. and Cosman, B. J. in Wilkins R. H., Rengachary S. S. (eds): Neurosurgery, New York, McGraw-Hill, Vol. III, pp. 2490-2498, 1984, and are hereby incorporated by reference herein in their entirety.

The Untied Stated Patent Application Publication entitled Method and Apparatus for Diagnosing and Treating Neural Dysfunction, by W. J. Rittman, Pub. No. US 2007/0032835 A1, Pub. Date: Feb. 8, 2007, describes an RF generator system comprising an RF generator with multiple active electrode output connections that enables the RF signal output the generator to be connected and delivered simultaneously to more than one electrode to deliver a therapeutic effect at each of the electrode positions at the same time. The RF generator's signal output is switched by switches and switch controllers so that the RF generator's output is applied to multiple needle-type treatment electrodes at the same time, and a reference electrode that does not have a specified treatment objective (such as a ground pad) is used as the path for return currents from the treatment electrodes. In another aspect, the switch and switch controller for one of the treatment electrodes performs independently from those of a second treatment electrode or from those of multiple individual treatment electrodes. This has one disadvantage that, because the same the signal output potential can be applied to more than one treatment electrode at the same time, the voltage of the generator's power supply and output electronics can be loaded down at the same time, causing sag or droop of the signal output voltage during application. Another disadvantage is that the electrical field from each of the treatment electrodes adds coherently in the bodily tissue, making it more difficult to separate their individual effects on the bodily tissue. Another disadvantage is that it makes it more difficult to control the RF signal output and to maintain the RF signal output so as to maintain the temperatures of the treatment electrodes at a set temperature chosen by the user. Another disadvantage is that no control objective, such as a target temperature value, is specified for the reference electrode. Another limitation is that inserted treatment electrodes are all connected via switches to the same output pole of the generator so that electric current does not flow between any two inserted treatment electrodes. Another limitation is that an indifferent reference electrode serves as the path for all return currents from all inserted electrodes. Another limitation is that the reference electrode is connected to one output pole of the generator for all steps in switching sequences produced by said system. Another limitation is that a switch is not specified for the reference electrode.

The use of radiofrequency (RF) generators and electrodes in neural tissue for the treatment of pain and functional disorders is well known. Included herein by reference, an as an example, the RFG-3C Plus RF Generator of Radionics, Inc., Burlington, Mass., and its associated electrodes are used in the treatment of the nervous system, and the treatment pain and functional disorders. The RFG-3C Plus generator has one electrode output jack for connection to a single active electrode, and it has one reference electrode jack for connection to a reference electrode. When the active electrode is inserted into the body, and the reference electrode is placed, typically on the patient's skin, then RF current form the RF generate flows through the patient's body between the two electrodes. The generator can be activated and its signal output can be applied between the electrodes. Typically, this is referred to as a monopolar configuration because the active electrode is of smaller area than the reference electrode, and so the concentration of RF current is highest near it and the action of the RF electric field, whether for heating or for pulsed RF field therapy is greater there. This usually referred to as a single electrode configuration since there is only one “active” electrode. Parameters that can be measured by the RFG-3C Plus RF generator include impedance, HF voltage, HF current, HF power, and electrode tip temperature. Parameters that may be set by the user include time of energy delivery, desired electrode temperature, stimulation frequencies and durations, and level of stimulation output. In general, electrode temperature is a parameter that may be controlled by the regulation of high frequency output power. Existing RF generators have interfaces that allow the selection of one or more of these treatment parameters, as well as various methods to display the parameters mentioned above.

In another example, the reference electrode can be inserted into the patient's body, and it can have an active area that is smaller and of comparable size to the active electrode. In that case, both electrodes become “active” in the sense that both of the electrodes have high temperature or electrical field effects on the tissues around them, so that they are both involved actively in the therapeutic effects the RF signal output. This can be referenced to as a single “bipolar configuration”.

A limitation for the monopolar and the bipolar configuration just described is that it limits the RF therapy to one or two electrode locations, respectively. In some situations it is desirable to treat more than one or two positions in the bodily tissue, and thus desirable to have more electrodes involved as the procedure goes on. For example, this can save time if there are multiple sites to be treated, as for example, multiple levels of the spinal medial branches to be treated for back pain.

The use of high frequency electrodes for heat ablation treatment of functional disease and in the destruction of tumors is well known. One example is the destruction of cancerous tumors of the kidney using radio frequency (RF) heat ablation. A paper by D. W. Gervais, et al., entitled “Radio Frequency Ablation of Renal Cell Carcinoma: Early Clinical Experience,” Radiology, Vol. 217, No. 2, pp. 665-672 (2000), describes using a rigid tissue perforating and penetrating electrode that has a sharpened tip to self-penetrate the skin and tissue of the patient. This paper is hereby incorporated by reference herein in its entirety.

Four patents have issued on PRF by Sluijter M. E., Rittman W. J., and Cosman E. R. They are “Method and Apparatus for Altering Neural Tissue Function,” U.S. Pat. No. 5,983,141, issued Nov. 9, 1999; “Method and System for Neural Tissue Modification,” U.S. Pat. No. 6,161,048, issued Dec. 12, 2000; “Modulated High Frequency Tissue Modification,” U.S. Pat. No. 6,246,912 B1, issued Jun. 12, 2001; and “Method and Apparatus for Altering Neural Tissue Function,” U.S. Pat. No. 6,259,952 B1, issued Jul. 10, 2001. These four patents are hereby incorporated by reference herein in their entirety.

United States patents by E. R. Cosman and W. J. Rittman, III, entitled “Cool-Tip Electrode Thermal Surgery System,” U.S. Pat. No. 6,506,189 B1, date of patent Jan. 14, 2003, and “Cluster Ablation Electrode System,” U.S. Pat. No. 6,530,922 B1, date of patent Mar. 11, 2003, described systems and method related to tissue ablation with radiofrequency energy and electrodes and are hereby incorporated by reference herein in their entirety.

In the prior art, the Cosman G4 Radiofrequency generator, in one mode of operation, switches radiofrequency electrical signal output among one, two, three, or four treatment electrodes such that a dispersive electrode carries all return currents from said treatment electrodes. This mode of operation can be referred as a “monopolar” mode. The energy delivered to each electrode can be adjusted to independently control one electrode-specific parameter for each electrode at the same time. In one sub-mode of operation, the said one electrode-specific parameter is the temperature measured at an electrode. In one sub-mode of operation, the said one electrode-specific parameter is the voltage applied between an electrode and the dispersive electrode. In one sub-mode of operation, the said one electrode-specific parameter is the output current flowing from an electrode. In one sub-mode of operation, the said one electrode-specific parameter is the power delivered to tissue due to signal output flowing from an electrode. One limitation of the prior art is that a reference electrode is used in addition to the four treatment electrodes. Another limitation is that the reference electrode is connected to signal output for all steps in all switching sequences whereby said treatment electrodes are connected to signal output.

In another mode of operation, the Cosman G4 Radiofrequency generator (Cosman Medical, Inc., Burlington, Mass.) connects radiofrequency electrical signal output to two treatment electrodes in a bipolar manner, without the use of a ground pad, such that each electrode serves as the path for return currents from the other electrode. In this bipolar configuration, said two treatment electrodes can be referred to a “bipolar pair”. Energizing two electrodes in a bipolar manner tends to focus the electrical current density and deposition of energy into the tissue between the two electrodes when said electrodes are close to each other. Energizing two electrodes in a bipolar manner can be used to create a “bipolar lesion” or “strip lesion”. Generation bipolar RF lesions is described in a paper by M. F. Ferrante, et al., entitled “Radiofrequency Sacroiliac Joint Denervation for Sacroiliac Syndrome”, Reg Anesth Pain Med 2001; 26(2):137-142, which is hereby incorporated by reference herein in its entirety. Generation bipolar RF lesions is described in a paper by C. A. Pino, et al., entitled “Morphologic Analysis of Bipolar Radiofrequency Lesions: Implications for Treatment of the Sacroiliac Joint”, Reg Anesth Pain Med 2005; 30(4):335-338, which is hereby incorporated by reference herein in its entirety. Generation bipolar RF lesions is also described in a paper by R. S. Burnham, et al., entitled “An Alternate Method of Radiofrequency Neurotomy of the Sacroiliac Joint: A Pilot Study of the Effect on Pain, Function, and Satisfaction”, Reg Anesth Pain Med 2007; 32(1):12-19, which is hereby incorporated by reference herein in its entirety. Generation bipolar RF lesions is also described in a paper by E. R. Cosman, Jr., et al., entitled “Bipolar Radiofrequency Lesion Geometry: Implications for Palisade Treatment of Sacroiliac Joint Pain”, Pain Practice 2010 (publication is currently pending), which is hereby incorporated by reference herein in its entirety. In the bipolar configurations described in the prior art, one parameter at a time is controlled independently by the energy delivered by the bipolar pair, and is hereby incorporated by reference herein in its entirety. In one sub-mode of operation, said one parameter is the maximum of the two temperatures measured at electrodes in a pair. In one sub-mode of operation, said one parameter is the RF voltage between the two electrodes in the pair. In one sub-mode of operation, said one parameter is the RF current flowing between the two electrodes in the pair. In one sub-mode of operation, said one parameter is the power delivered to tissue delivered by signal output delivered to the pair of electrodes. One limitation of the prior art is that the temperatures measured at each electrode in a bipolar pair are not controlled substantially independently. Another limitation of the prior art is that the temperature of one electrode in a bipolar pair can be substantially below a target set temperature for a substantial portion of the total treatment time; one example of a explanation for this phenomenon is that the electrical current flowing through one electrode in a bipolar pair is substantially the same as the electrical current flowing through the other electrode in a bipolar pair, because both electrodes in a bipolar pair serves as a path for return current for the other electrode. Another limitation is that the power delivered by one electrode in a pair is the same as the power delivered as the other electrode in said pair. Another limitation is the signal output for one electrode in the bipolar pair is not controlled independently of the other. Another limitation is that the voltage for one electrode is not controlled independently of the other. Another limitation is that the current for one electrode is not controlled independently of the other. Another limitation is that the power for one electrode is not controlled independently of the other.

In another mode of operation, four electrodes, labeled “E1”, “E2”, “E3”, and “E4”, are placed in tissue and connected to the Cosman G4 Radiofrequency generator. In this mode of operation, the generator produces a sequence of switch states, where the switch states can take one of three forms at any one time. In the first said form of switch states, E1 and E2 are connected to opposite poles of an RF power supply and E3 and E4 and disconnected from signal output. In the second said form of switch states, E3 and E4 are connected to opposite poles of an RF power supply and E1 and E2 are disconnected from signal output. In the third said form of switch states, all electrodes are disconnected from signal output. As such the generator produces energizes fixed, disjoint pairs of electrodes, E1-E2 and E3-E4, in sequence, where each pair is energized in a bipolar manner and where each electrode in a pair serves as the path for return currents for the other electrode in the pair, for the entire duration of the operational mode. The energy delivered to each pair is adjusted to independently control one pair-specific parameter for each pair of the other pair's pair-specific parameter. In one sub-mode of operation, said one pair-specific parameter is the maximum of the temperatures measured at each electrode in a pair. In one sub-mode of operation, said one pair-specific parameter is the voltage between the two electrodes in a pair. In one sub-mode of operation, said one pair-specific parameter is the current flowing between electrodes in a pair. In one sub-mode of operation, said one pair-specific parameter is the power delivered to tissue by a pair. One limitation of the prior art is that temperature is not independently controlled at all four electrodes at the same time. Another limitation of the prior art is that an electrode-specific parameter, such as the temperature measured at an electrode, is not controlled for each electrode at the same time in a manner that is substantially independent of the electrode-specific parameters associated with all other electrodes. Another limitation is that a parameter that is a function of the signal applied to one electrode over more than one switching step, is not independently controlled for each electrode. Another limitation is that the root-mean-square (RMS) voltage applied to one electrode over a duration containing more than one switching step, is not independently controlled for all electrodes. Another limitation is that the root-mean-square (RMS) current applied to one electrode over a duration containing more than one switching step, is not independently controlled for all electrodes. Another limitation is that the average power applied to one electrode over a duration containing more than one switching step, is not independently controlled for all electrodes. Another limitation of the prior art is that the temperature of one electrode in each bipolar pair may be substantially below a target set temperature for a substantial portion of the total treatment time; one example of a reason for why this can occur is that the electrical current flowing through one electrode in a bipolar pair is substantially the same as the electrical current flowing through the other electrode in a bipolar pair, because both electrodes in a bipolar pair serves as a path for return current for the other electrode. Another limitation is that at most two electrodes are connected to signal output in all steps of sequences by which electrodes are connected and disconnected from signal output.

In the prior art, the Cosman G4 radiofrequency generator can be switched between monopolar and bipolar modes operation by manual action of the user, said actions including changing controls in the user interface of the generator, and said actions including attaching electrodes and dispersive ground pads into external jacks of the generator. Such mode switching is not automated and requires a duration that is very long relative to the time in which a single set of switch states is held before it is changed in an automated manner during mode of automated multi-electrode control. A limitation of this prior art is that the Cosman G4 is not configured to automatically generate a sequence of connections between electrodes and system output poles that includes a step in which three or more electrodes are connected to system output poles at the same time, and in which a reference ground pad is not persistently connected to a reference output pole for the purpose of collecting return currents from other electrodes. Another limitation of this prior art is that the Cosman G4 is not configured to rapidly generate a sequence of connections between electrodes and system output poles that includes a step in which three or more electrodes are connected to system output poles at the same time, and in which a reference ground pad is not persistently connected to a reference output pole for the purpose of collecting return currents from other electrodes. Another limitation of the prior art is the temperature at each electrode is not controlled at the same while delivering electrical current between arbitrary connections of electrodes and ground pads to generator output poles. Another limitation of the prior art is an electrode-specific parameter, such as average power, at each electrode is not controlled at the same while delivering electrical current between arbitrary grouping of electrodes and the ground pad.

In the prior art, the Neurotherm SimplicityIII probe (Neurotherm, Wilmington, Mass.) has three metallic elements which are integrated into a single elongated probe, such that each metallic element is electrically insulated from the other elements, and such that each metallic element can be connected to system power supplies independently. Each of these metallic elements constitutes a treatment electrode and can be referred to as “E1”, “E2”, and “E3”, respectively. When the Simplicity III probe is connected to the Neurotherm NT1100 radiofrequency generator and is placed in the sacroiliac region of a body, and a reference ground pad “GP” is attached to the NT 1100 and to the said body, a non-repeating automatic sequence is produced. In one step of the sequence, E1 and E2 are energized in a bipolar manner, with E3 and the ground pad disconnected, and maximum of the temperatures at E1 and E2 is controlled to produce a heat lesion. In another step of the sequence, E2 and E3 are energized in a bipolar manner, with E1 and the ground pad disconnected, and maximum of the temperatures at E2 and E3 is controlled to produce a heat lesion. In another step of the sequence, the ground pad carries return currents from the electrode E1, with electrodes E2 and E3 disconnected, and the temperature at E1 is controlled to produce a heat lesion. In another step of the sequence, the ground pad carries return currents from the electrode E2, with electrodes E2 and E3 disconnected, and the temperature at E2 is controlled to produce a heat lesion. In another step of the sequence, the ground pad carries return currents from the electrode E3, with electrodes E2 and E1 disconnected, and the temperature at E3 is controlled to produce a heat lesion. One limitation of the prior art is that the steps of this sequence typically have duration on the order of 1 to 1.5 minutes or greater. Another limitation of the prior art is that the steps of this sequence do not have duration that is small relative to the thermal dynamics of an electrode when they are placed in a living body. Another limitation of the prior art is the temperatures measured at each of the three treatment electrodes are not controlled independently of each other at the same time. Another limitation of the prior art is the temperature of one electrode in a bipolar pair may be substantially below a target set temperature for a substantial portion of the phase of the sequence in which that pair is energized; one example of a reason for this phenomenon is that the electrical current flowing through one electrode in a bipolar pair is substantially the same as the electrical current flowing through the other electrode in a bipolar pair, because both electrodes in a bipolar pair serves as a path for return current for the other electrode. Another limitation of the prior art is that at most two electrodes are connected to signal output at once during the entirety of the sequence. Another limitation of the prior art is that no electrode-to-ouput-pole configuration is repeated in another step. The Neurotherm NT1100 can be manually switched to another mode of operation, the “SimplicityII” mode, in which the automatic sequence only includes the E1-E2, E1-GP, and E2-GP steps. The Neurotherm NT1100 can be manually switched to another mode of operation in which one, two, or three treatment electrodes are placed in a living body and energized in a monopolar configuration such that a reference ground pad carries the return currents from all treatment electrodes. The Neurotherm NT1100 can be manually switched to another mode of operation in which two treatment electrodes can be energized in a bipolar configuration. The time to switch between the monopolar, bipolar, SimplicityII, SimplicityIII, and other output modes is longer than 5 seconds and in typical use is not performed in less than 10 seconds. A limitation of this prior art is that the Neurotherm NT1100 is not configured to automatically generate a sequence of connections between electrodes and system output poles that includes a step in which three or more electrodes are connected to system output poles at the same time, and in which a reference ground pad is not persistently connected to a reference output pole for the purpose of collecting return currents from other electrodes. Another limitation of this prior art is that the Neurotherm NT1100 is not configured to rapidly generate a sequence of connections between electrodes and system output poles that includes a step in which three or more electrodes are connected to system output poles at the same time, and in which a reference ground pad is not persistently connected to a reference output pole for the purpose of collecting return currents from other electrodes.

In the prior art, multiple, non-temperature-sensing electrodes are placed in porcine or human liver and energized by rapidly duty-cycling between pairs of said electrodes, in order to control the power delivered to each pair of electrodes, in order to reduce blood flow in a region of the liver to facilitate tissue resection or ablate tumors. This prior art is described in the following publications, which are hereby incorporated by reference herein in their entirety: a paper by D. Haemmerich, et al., entitled “A device for radiofrequency assisted hepatic resection”, Proceedings of the 26th Annual International Conference of the IEEE EMBS, Sept. 1-5, 2004: 2503-2506; a paper by I. dos Santos I, et al., entitled “A surgical device for radiofrequency ablation of large liver tumors”, Physiol. Meas. 2009; 29: N59-N70.; a paper by D. J. Schutt, et al., entitled “An electrode array that minimizes blood loss for radiofrequency assisted hepatic resection”, Med Eng Phys. 2008 May; 30(4): 454-459; and a paper by R. M. Striegel entitled “An Electrode Array for Limiting Blood Loss During Liver Resection: Optimization via Mathematical Modeling”, The Open Biomedical Engineering Journal 2010; 4:39-46. One limitation of the prior art is that rapid duty-cycling among multiple electrode is not configured for control of a measured temperature. One limitation of the prior art is that rapid duty-cycling among multiple electrode is not configured for control of a temperature at each electrode. Another limitation to the prior art is that rapid switching between electrode pairs is not configured to control a different parameter for each electrode, such as the average power delivered to a specific electrode over a duration longer than one step of the duty-cycling. Another limitation to the prior art is that rapid switching between electrode pairs is not configured to control a different parameter for each electrode, such as an aggregate measure of current delivered to a specific electrode over a duration longer than one step of the duty-cycling. Another limitation to the prior art is that rapid switching between electrode pairs is not configured to control a different parameter for each electrode, such as an aggregate measure of voltage delivered to a specific electrode over a duration longer than one step of the duty-cycling. Another limitation of the prior art is that rapid duty-cycling among multiple adjacent electrodes is not performed in the sacroiliac region, nor for ablating nervous tissue, nor for managing pain. Another limitation of the prior art is no more than two electrodes are energized in any step of the duty-cycle switching process. Another limitation is that the impedance between pairs of electrodes is not controlled. Another limitation is that voltage between pairs of electrodes is not controlled. Another limitation is that the current between pairs of electrodes is not controlled. Another limitation is the water content of tissue is not controlled. Another limitation is that the blood flow tissue is not controlled.

In a paper by A. Hacker, et al., “Technical characterization of a new bipolar and multipolar radiofrequency device for minimally invasive treatment of renal tumours”, BJU International 2006; 97: 822-828, two, four, or six electrodes are placed in the porcine or human kidney, and pairs of said electrodes are energized in a bipolar manner sequentially, where each pair is energized one after another for a specific period of 3 seconds, and where the output level applied to each pair is adjusted to control either a single impedance or a single resistance measured between the two output poles of a radiofrequency power supply which are sequentially to connected to exactly two electrodes at a time. One limitation of the prior art is that duty-cycling among multiple electrode is not configured for control of a measured temperature. Another limitation to the prior art is that rapid switching between electrode pairs is not configured to control independently an electrode-specific parameter for each electrode, such as the power delivered to a specific electrode. Another limitation to the prior art is that rapid switching between electrode pairs is not configured to control an impedance for each pairing of electrodes. Another limitation to the prior art is that rapid switching between electrode pairs is not configured to control an impedance for each electrode, where each the impedance for a given electrode is measured between said electrode and a reference structure, such as another electrode or set of electrodes connected to a reference potential. Another limitation of the prior art is the respective impedances between each pairing of electrodes are not controlled independently. Another limitation of the prior art is that rapid duty-cycling among multiple adjacent electrodes is not performed in the sacroiliac region, nor for ablating nervous tissue, nor for managing pain. Another limitation of the prior art is no more than two electrodes are energized in every step in the duty-cycle switching process. Another limitation is that the duration of connection to each pair of electrodes is a fixed duration. Another limitation is that the output level is adjusted to control only one parameter at a time.

The present invention overcomes the stated and other limitations of the prior art.

### SUMMARY OF THE INVENTION

In one exemplary embodiment, the present invention is directed towards systems and methods for ablating tissue in the living body, including use of multiple probes comprising high frequency electrodes and temperature-sensing probes.

In another example of the present invention, a system for ablating tissue includes more than two electrodes that are inserted into a patient's tissue and includes an RF generator that can apply output signal from the generator and a control system that can monitor the temperature of the electrode, adjust the amplitude, distribution, and timing of the of the output signal in a bipolar manner across at least two subgroups of electrodes at a given time so as to achieve a uniform temperature distribution on the inserted electrodes.

In another example of the present invention, a system for ablating tissue includes more than two electrodes that are inserted into a patient's tissue and includes an RF generator that can apply output signal from the generator and a control system that can monitor the temperature of the electrode, adjust the amplitude, distribution, and timing of the of the output signal in a bipolar manner across at least two subgroups of electrodes at a given time so as to achieve a desired temperature distribution on the inserted electrodes.

In another example of the present invention, a system for ablating tissue using a multiplexing system so that the output of a high frequency generator can be applied in a bipolar configuration across a first subgroup of n electrodes and a second subgroup of m electrodes that are subgroups of a total of N electrodes inserted into a patient's body, where N is an integer greater than two. A control system can vary the amplitude, time duration, and the distribution of n and m so that the respective temperatures of the N electrodes can each be held at a desired temperature.

In another example of the present invention, a system and method includes at least three electrodes inserted into a patient's body and a high frequency generator that can be connected to the electrode through a switching and control system so that the output signal of the generator can be connected to subgroups of the electrodes in a distributions of time and of electrode combinations to achieve a constant temperature on the electrodes.

In another example of the present invention, a system and method can include in addition to at least two electrodes inserted into the patient's body, a ground electrode or reference electrode in contact with the patient's skin or other anatomy remote of the treatment location, that can also be introduced into the switching and control system so that the reference electrode can be used at controlled times and durations as one of a bipolar pair involving the reference electrode as one of the pairs and one or more of the inserted electrode as the other pair of the bipolar configuration. In one more specific example, said system and method can be used to control the temperature measured at each of the said at least two electrodes inserted into the patient's body. In another more specific example, said system and method can be used to control the power delivered to each of the said at least two electrodes.

In another example of the present invention, a system and method for applying electrical output signals to tissue can include at least three electrodes that are placed in or on a patient's body, and includes a control system that can produce a sequence of operational states of an electrical signal generator; where said electrical signal generator includes at least two output poles capable for producing different electrical potentials; where a step of said sequence of operational states can be configured to connect each of at least two disjoint subsets of electrodes to a different output pole of the electrical signal generator; where in at least one step of said sequence, the total number of inserted electrodes connected to any output pole is at least three; where there is no designated reference electrode that carries substantial return current from other electrodes during every step of the said sequence of operational states; where electrical signal applied to each output pole and/or the duration of a step and/or the assignment of electrodes to poles can be adjusted by the controller during and/or between each step of said sequence of operational states. In one more specific example, the said controller can produce said sequence of operational states in a fully automated manner. In one more specific example, the said controller can produce said sequence of operational states in a rapid manner. In one more specific example, the said controlled can produce said sequence of operational states, where each step of said sequence has a duration of less than one minute. In one more specific example, the said controller can produce said sequence of operational states, where each step of said sequence has a duration of less than five seconds. In one more specific example, the said controlled can produce said sequence of operational states, where each step of said sequence has a duration of less than one second. In one more specific example, the said controlled can produce said sequence of operational states, where each step of said sequence has a duration of less than 250 milliseconds. In one more specific example, the said controlled can produce said sequence of operational states, where each step of said sequence has a duration of less than 100 milliseconds. In one more specific example, the sequence can be configured to use electrical signal output to control other parameters at the same time, substantially independently of each other, where the number of other parameters is at equal to the total number of electrodes connected to the system. In another more specific example, the sequence can be configured to control the temperature of tissue near each electrode at the same time. In another more specific example, the sequence can be configured to control simultaneously the average power delivered to each electrode over a window of time that contains more than one step in the sequence.

In another example of the present invention, a system and method can deliver electrical signal output sequentially to subsets of at least three electrodes placed in bodily tissue for the purpose controlling a parameter for each of said at least three electrodes, where control of all parameters is achieved at the same time, and where each parameter is controlled substantially independently of all other parameters. One advantage of this aspect of the present invention that a substantially independent parameter can be controlled for each of said at least three electrodes at the same time without the use of an additional electrode.

In another example of the present invention, a system and method can deliver electrical signal output sequentially to subsets of at least three electrodes placed in bodily tissue for the purpose of controlling multiple parameters, where the number of said multiple parameters is at least the number of said at least three electrodes, where control of all parameters is achieved at the same time, and where each parameter is controlled substantially independently of all other parameters. One advantage of this aspect of the present invention is that more independent parameters can be controlled than the number of electrodes.

In another example of the present invention, a system and method can switch electrical energy among at least two electrodes placed in tissue of a body and at least one additional electrode placed in contact with the said body for the purpose controlling a parameter for each of said at least two electrodes, where control of all parameters is achieved at the same time, and where each parameter is controlled substantially independently of all other parameters. One advantage of this aspect of the present invention is that a substantially independent parameter can be controlled for each of said at least two electrodes at the same time by delivery of electrical energy, where said electrical energy is delivered only to the said two electrodes for some duration of the time into which electrical energy is delivered. Another advantage of this aspect of the present invention that delivery of electrical energy can be focused in a tissue region between the said at least two electrodes, while at the same time independently and simultaneously controlling a parameter for each of said two electrodes.

In another example of the present invention, a system and method can switch electrical energy among at least two electrodes placed in tissue of a body and at least one additional electrode placed in contact with the said body for the purpose controlling multiple parameters, where the number of said multiple parameters is at least the number of said at least two electrodes, where control of all parameters is achieved at the same time, and where each parameter is controlled substantially independently of all other parameters. One advantage of this aspect of the present invention is that more independent parameters can be controlled than the number of the said at least two electrodes. Another advantage is that the operating conditions under which control of all said parameters are achievable can be expanded relative to the case where the said at least one additional electrodes are not used.

In another example of the present invention, a system and method can switch electrical energy among at least three electrodes inserted into bodily tissue where for at least one step of a switching sequence more than two electrodes are connected to generator signal output at the same time. In one more specific example, at least two of the said at least three electrodes can be integrated into different physical structures. In one more specific example, all said at least three electrodes can be integrated in the same physical structure. One advantage of this aspect of the present invention is additional patterns of electrical energy delivery are possible when more than two inserted electrodes are energized for some duration of an overall energy-delivery period, than are possible if at most two electrodes are energized at any time during an overall energy-delivery period.

Advantages of the system and method of the present invention include the ability to heat multiple electrodes in the same clinical intervention using multiple and multiplexed bipolar configurations so that uniform temperature can be achieved on the electrodes.

In another aspect, an advantage of the system and method of the present invention is that multiple electrodes can be placed and heated in a simultaneous, or nearly simultaneous, process while avoiding the differences of impedance characteristics that would cause non-uniform heating in the case of standard bipolar RF application wherein difference in tissue impedance would cause runaway heating on one electrode of a bipolar pair.

Another advantage is that use of varied numbers of subsets of the electrodes in the bipolar pairs enables control of the balance in thermal heating around several electrodes at the same time. This has an advantage, for example, in the application of pain therapy of the sacroiliac (SI) joint where it is an advantage to be able to simultaneously heat several electrodes in one procedure process. This can save time for the clinical and provide a more complete and uniform thermal lesion to be made over a large area of innervations as in the SI joint.

Another advantage is that multiple bipolar electrodes heating can produce a more complete and greater heating between the electrodes than multiple monopolar heating for comparable electrode spacing.

The invention can be used in numerous organs in the body, including the brain, spine, liver, lung, bone, kidney, abdominal structures, etc., and for the treatment of cancerous tumors, functional disorders, pain, tissue modifications, bone and cartilage fusions, and in cardiac ablation.

The detail of one or more embodiments of the invention are set forth in the accompanying drawings and description below. Other features, objects and advantages of the invention will be apparent from the description and drawings and from the claims.

### BRIEF DESCRIPTION OF THE DRAWINGS

In the drawings that constitute a part of the specification, embodiments exhibited various forms and features hereof are set forth, specifically:

FIG. 1 is a schematic diagram showing percutaneous placement of electrodes into the tissue of a patient's body, a ground electrode, and a high frequency generator and switching control system.

FIG. 2 is a schematic diagram showing a sequence of bipolar, multipolar, and multiplexing procedure using three high frequency electrodes.

FIG. 3 is a schematic diagram showing a sequence of bipolar, multipolar, and multiplexing procedure using four high frequency electrodes.

FIG. 4 is a schematic flow chart of a process of bipolar multiplexing on electrodes inserted into the patient's body.

FIG. 5 is a schematic flow chart of a process of bipolar multiplexing on electrodes inserted into the patient's body and including the use of a reference electrode.

FIG. 6 is a schematic flow chart of a process of bipolar multiplexing on electrodes inserted into the patient's body and including the use of a reference electrode.

FIG. 7A is a schematic diagram showing a system for bipolar, multipolar, and multiplexing procedure using multiple high frequency electrodes.

FIG. 7B is a schematic diagram showing a system for bipolar, multipolar, and multiplexing procedure using multiple high frequency electrodes.

FIG. 8 is a schematic diagram in block diagram form showing a system for bipolar, multipolar, and multiplexing procedure using multiple high frequency electrodes.

FIG. 9 is a schematic flow chart of a process of bipolar multiplexing on electrodes inserted into the patient's body and including the use of a reference electrode.

FIG. 10 is a schematic diagram showing a sequence of bipolar, multipolar, and multiplexing procedure using multiple electrodes.

FIG. 11 is a schematic diagram showing a sequence of bipolar, multipolar, and multiplexing procedure using multiple electrodes.

FIG. 12 is a schematic diagram showing a sequence of bipolar, multipolar, and multiplexing procedure using multiple electrodes.

FIG. 13 is a schematic diagram showing a sequence of multipolar, and multiplexing procedure using multiple electrodes.

FIG. 14 is a schematic diagram showing bipolar, multipolar, and multiplexing procedure using multiple electrodes inserted into the SI joint region.

FIG. 15 is a schematic diagram showing a sequence of bipolar, multipolar, and multiplexing procedure using multiple electrodes inserted into the spinal region.

FIG. 16 is a schematic diagram showing a sequence of bipolar, multipolar, and multiplexing procedure using multiple electrodes.

FIG. 17 is a schematic diagram showing a sequence of bipolar, multipolar, and multiplexing procedure using multiple electrodes.

FIG. 18 is a schematic diagram showing a sequence of bipolar, multipolar, and multiplexing procedure using multiple electrodes inserted into target volume such a tumor or internal organ target region.

FIG. 19 is a schematic diagram showing a sequence of bipolar, multipolar, and multiplexing procedure using multiple electrodes inserted into target volume such a tumor or internal organ target region.

FIG. 20 is a schematic diagram showing a sequence of bipolar, multipolar, and multiplexing procedure using multiple electrodes inserted into target volume such a tumor or internal organ target region.

FIG. 21 is a schematic diagram showing a sequence of bipolar, multipolar, and multiplexing procedure using multiple electrodes inserted into target volume with multiple electrode contacts on a curva-linear structure.

FIG. 22 is a schematic diagram showing a sequence of bipolar, multipolar, and multiplexing procedure using multiple electrodes.

FIG. 23 is a schematic diagram showing an array of bipolar, multipolar, and multiplexing procedure using multiple electrodes.

FIG. 24 is a schematic diagram showing an array of bipolar, multipolar, and multiplexing procedure using multiple electrodes.

FIG. 25 is a schematic diagram showing an array of bipolar, multipolar, and multiplexing procedure using multiple electrodes.

FIG. 26 is a schematic diagram showing an array of bipolar, multipolar, and multiplexing procedure using multiple electrodes.

FIG. 27 is a schematic diagram shown an array of electrodes configured to control parameters whose number is greater than the number of said electrodes.

FIG. 28 is a schematic diagram showing percutaneous placement of electrodes into the tissue of a patient's body, a ground electrode, and a high frequency generator and switching control system.

FIG. 29 is a schematic diagram of a sequence of bipolar multiplexing on electrodes inserted into the patient's body, including the use of a reference electrode. In the sequence, there is a step in which each of three electrodes are attached to one of the two system output poles, and there is a step in which the reference electrode is not the path for return currents for other electrodes.

FIG. 30 is a schematic diagram of a sequence of bipolar multiplexing on electrodes inserted into the patient's body, not including the use of a reference electrode. In the sequence, there is a step in which each of three electrodes are connected one of the two system output poles at the same time.

FIG. 31 is a schematic diagram of a sequence of multipolar multiplexing on electrodes inserted into the patient's body, not including the use of a reference electrode. In the sequence, there is a step in which each of three electrodes are connected one of the three system output poles at the same time.

FIG. 32 is a schematic diagram showing a sequence of bipolar, multipolar, and multiplexing procedure using two high frequency electrodes and a reference electrode to control an electrode-specific parameter for each of the two high frequency electrodes at the same time.

FIG. 33 is a schematic diagram showing a sequence of bipolar, multipolar, and multiplexing procedure using three high frequency electrodes to control simultaneously a parameter for each electrode.

FIG. 34 is a schematic diagram showing a sequence of bipolar, multipolar, and multiplexing procedure using four high frequency electrodes to control simultaneously more independent parameters than the number of electrodes.

FIG. 35 is a schematic diagram showing a sequence of bipolar, multipolar, and multiplexing procedure using three high frequency electrodes to control a parameter for each electrode at the same time by means of varying the duration of each step in the sequence.

FIG. 36 is a schematic diagram showing percutaneous placement of electrodes into the tissue of a patient's body, a ground electrode, and a high frequency generator and switching control system. A probe is shown that houses two electrodes that redundantly control the same parameter, such as a temperature.

### DETAILED DESCRIPTION OF THE INVENTION

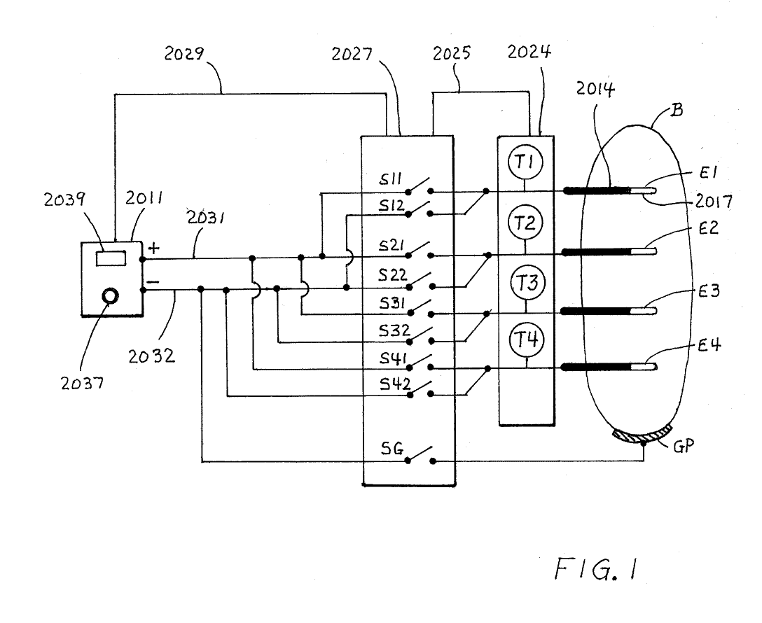

Referring to FIG. 1, an RF generator 2011 that can be connected to multiple electrodes E1, E2, E3, and E4 is shown. Signal output from the generator can connect to output jacks designated by the symbols + and − by the cables 2031 and 2032. The designation + and − are schematic and represent the two output poles of the high frequency generator 2011 between which the signal output of the generator 2011 is impressed. In one example where the output of the generator is an alternative signal output, for example a high frequency signal output, the output signal on both the + and − poles can alternate between positive and negative values; in that example, the + and − designations do not necessarily imply that the output signal on the poles are positive and negative, respectively. Electrodes E1, E2, E3, and E4 can be connected in a controlled way to the jacks + and − through the control unit 2027. Unit 2027 comprises switches designated S11, S12, S21, S22, S31, S32, S41, and S42 that enable the signal output to be switched among the electrodes. The control unit 2027 insures that the switches S11 and S12 are not closed at the same time to prevent shorting out of the outputs + and −. The same is true for the other switch pairs S21 and S22, S31 and S32, and S41 and S42. The unit 2027 can switch any combination of bipolar pairs of electrodes across the outputs + and − according to a control algorithm or electronic sequence control in unit 2027. For example, closing S11 and S22 will put output + on E1 and − on E2. Or, for example, closing S11 and S21 and closing S32 and S42 will put the + output on E1 and E2 and the − output on E3 and E4. In this way, the bipolar output + and − from generator 2011 can be put across any combination of pairs of the electrodes E1, E2, E3, E4. In this way, the output from generator 2011 can be applied to arbitrary combinations of electrodes E1, E2, E3, E4, with one or more electrodes connected to the + output, and one or more electrodes connected to the − output. This can be referred to herein as “bipolar multiplexing”.

Referring to FIG. 1, in another example, the ground pad GP can also be part of the process of applying energy to tissue as another electrode that can be switched in by switch SG. For example, as part of the sequence of electrode combinations, the unit can perform multiplexing bipolar connections as described above and it can also switch in the ground pad as part of the sequence so that GP can become another of the active electrodes that can be used by the units 2027 to achieve a desired temperature on the inserted electrode E1, E2, E3, E4. The ground pad GP can also be referred to as a reference electrode, for example, if it is connected to the − output pole of the generator 2011. The ground pad GP can also be referred to as an indifferent electrode. In another example, a connection between the GP and the + output pole can be made via an additional switch, so that the GP be connected to either the + or the − output pole. In one example, a ground pad GP can take the form of an area gel pad. In one example, a ground pad GP take the form of a conductive plate. In one example, a ground pad GP can take the form of a probe inserted into a non-treatment region of the body. In one example, a ground pad GP take the form of a conductive plate. In one example, a ground pad GP can be capacitively coupled to bodily tissue. In one example, a ground pad GP can be resistively coupled to bodily tissue. In one example, a ground pad can take one of a number of different forms which should be familiar to one skilled in the art. Then current output from the unit 2011 can run from the inserted electrodes E1, E2, E3, E4 to the GP. GP is typically a conductive area electrode that is in electrical contact with the skin of the patient's body B. Thus a combination of multiplexing bipolar connections can be a mixture of connections to inserted electrodes alone or, in another example, and can include connection to the GP another electrode as the controller is configured in FIG. 1.

Referring to FIG. 1, a system of four electrode probes E1, E2, E3, E4, are shown in inserted into tissue of a living body B through the skin. The probes can be high frequency electrodes having elongated shafts that are configured to be directed into the body and bodily organs to a target region, for example, the region around the spinal nerves or the SI joint. The probes can comprise a metal or a plastic tube. The tubes can be flexible or rigid, depending on the clinical application. The electrodes can be elongated shafts that are partially insulated as illustrated by the black area 2014 on E1, and can have an exposed conductive tip 2017 inside of which can be a temperature measuring sensor such as a thermocouple. The temperature of the tissue around each electrode and thereby be measured by detectors T1, T2, T3, T4, respectively, and the temperature information can be fed into the control unit by the cable 2025. The control unit can use this thermal information from each electrode for example, to produce a switching sequence to achieve a constant temperature on each of the electrodes during the process of high frequency heating of the tissue around the electrodes.

In one example, a desired high frequency voltage amplitude V(RF) can be produced by generator 2011 and the signal outputs at the output jacks + and − will be voltage +V and −V, and this voltage will oscillate at the frequency of the high frequency generator 2011. This oscillating signal output can then be connected to the exposed conductive tips of the bipolar pairs of electrodes via the switches at any given time and in a sequence that is controlled by unit 2027. In one example, the Voltage amplitude V can be controlled by a manual control 2037 unit 2011. In another example, it can be controlled by the control unit 2027, the control signal being fed back into the generator 2011 by the connection 2027. During the process of tissue heating, the voltage V can be adjusted so that a desired temperature is reached on all the electrodes.

In another example, referring to FIG. 1, the signal output from the generator 2011 can be chosen according to clinical needs and can be varied in coordination with the switching process controlled by 2027 to achieve a desired thermal distribution around the electrodes in accordance with clinical objectives. In one example, as the switched connections to the electrodes are being made, the voltage +V and −V from the output jacks + and − on unit 2011 can be adjusted to increase or decrease the heating on the connected electrodes so as to have the thermal distribution converge on a desired objective, and that objective can be to achieve a desired size of lesion volume or to modify, alter the shape of, enlarge, or modify the lesion volume. For example, as the unit 2027 switches to one set or combination of bipolar electrodes, the voltage output can be V1. When controller 2027 switches to another set of bipolar electrodes, the voltage from unit 2011 can be changed to another value V2. That variation or change of the voltage output corresponding to different switch positions among the electrodes can be used to tailor the temperatures on the multiple electrodes so as to converge to a desired overall temperature distribution on the electrodes. That objective can be, for example, to equalize the temperatures at all of the electrodes to achieve a desired targeted value. In another example, the objective can be to have a desired non-uniform temperature distribution among the electrodes so as to shape the heat lesion volume as desired. In another example, the objective can be to bring each electrode's temperature into a range which is particular to that electrode, and where the range can either vary over time or be constant over time. In another example, the output signal can be chosen to be the electrical current I from the outputs + and −, and that current I can be modulated as the controller 2027 switches connection to the multiplexed bipolar electrodes.

Referring to FIG. 1, in another example, the number of electrodes can be different from the number of four electrodes as schematically depicted in the FIG. 1. In one example, a more general case and embodiment can comprise the number N of inserted electrodes, where N can be and number greater that three. The corresponding electrodes can be designated as E1, E2, . . . , E(N−1), and EN, analogous to designation as for the four electrodes in FIG. 1. The controller can correspondingly comprise a more general set of switches S11, S12, S21, S22, . . . , SN1, and SN2. The thermal readout can be designated T1, T2, . . . , TN, if all of the electrodes have built-in temperature sensors. In one example of this embodiment, there can be no ground pad GP present, and in that case, the inserted electrodes E1 through EN can be activated in a multiplexed bipolar manner as described above and in the further embodiments described herein. In another example, a ground pad GP can be present, and it can become part of the multiplexed bipolar switching combinations as describe in related embodiments herein. The number N of electrodes can be chosen to accommodate clinical needs. For example, for making linear lesions in the case of the SI joint, N=3, 4, 5, 6, 7, or even more electrodes can be used to make a sufficiently large “strip” lesion to denervate some or all of SI joint. In another example, for devascularizing the kidney preparatory to hemi-nephrectomy procedures, N=3, 4, 5, 6, 7, 8, or more electrodes may have to be inserted to produce a coagulative “palisade” barrier to reduce bleeding risk during surgical resection of the kidney lobe. In another example, non-linear arrays of inserted electrodes can be used so that a desired lesion volume can be achieved, or a shaped lesion volume can be achieved according to clinical needs.

Referring to FIG. 1, in another example, the controller 2027 can execute a switching sequence in each step of which a subset of n electrodes of the N inserted electrodes is connected to the + output pole and a subset of m electrodes of the N inserted electrodes in connected to the − output pole, where n is an integer greater than zero and where m is an integer greater than zero. In one example of the present invention, the sum of n and m can be greater than two for at least one step is said switching sequence. In one example of the present invention, the sum of n and m can be greater than two for at least one step is said switching sequence, and the temperature at T1, . . . , TN is being controlled. In another example of the present invention, the sum of n and m can be greater than two for at least one step of said switching sequence and the detectors T1, . . . , TN can be omitted from the system. In another example of the present invention, the sum of n and m can be greater than two for at least one step of said switching sequence and the detectors T1, . . . , TN can have no influence on the delivery of output to the N inserted electrodes. An advantage of a system where n+m>2 for at least one step of said switching sequence is that the controller can have more flexibility in achieving clinical objectives than it would in a system were n+m=2 for all steps in said switching sequence.

Referring to FIG. 1, in another example, the generator 2011, the switching control unit 2027, and the measurement readout 2024 can either be housed in the same physical chassis or can be housed in separate physical chassis and connected with cables.

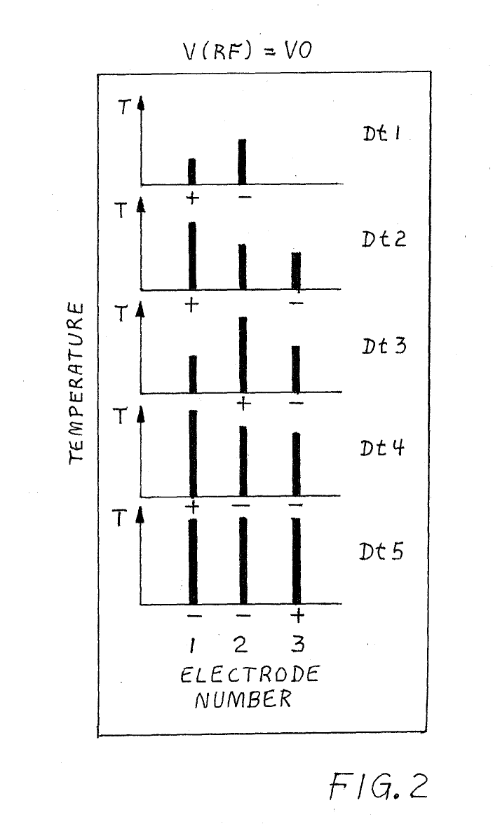

Referring to FIG. 2, in one example, a sequence of switching combinations and resulting temperature readings is schematically shown for the case of N=3 inserted electrodes in the patient's body. In this example, n refers to the number of electrodes E1, E2, E3 connected to one pole of the output of a generator 2011, and m refers the number of electrodes E1, E2, E3 connected to the other output of that generator 2011. For example, n can refer to the number of electrodes connected to the + output and m can refer to the number of electrodes connected to the − output. FIG. 2 illustrates multiplexing bipolar switching in which combinations of bipolar pairs with single electrodes on each pole, that is n=1 and m=1, are used, and in which bipolar combinations are used with the number of activated electrodes in one of the bipolar poles in greater one, that is n=1 and m=2. Schematically, the time intervals Dt1, Dt2, etc. represent the time durations for which the switched-in bipolar combinations are connected to the signal outputs + and − of the high frequency generator 2011 in FIG. 1. For example, in the time period Dt1, electrodes E1 and E2 are switched in, and the + signal output pole of generator 2011 is connected to E1, and the − output pole of 2011 is connected to E2. This is schematically illustrated by the + symbol beneath the E1 temperature histogram, and the − symbol beneath the E2 histogram. In one example, during that time interval Dt1, the signal output is maintained at V(RF)=V0 (where the “RF’ stands for radiofrequency signal output from 2011). The FIG. 2 illustrates that during Dt1, E1 heats the tissue somewhat less than E2, i.e., the temperature measured by the temperature sensor in E1 as detected by the readout T1 measures a lower temperature than that of E2 during that interval Dt1. The difference in heating at the two electrodes can be an intrinsic physical characteristic of the tissue impedance around each electrode, and for a given pair of bipolar electrodes this becomes a problem to overcome if, for example, a uniform temperature distribution is desired. It is one objective of the present patent to overcome this problem by using the multiplexing bipolar sequence and the proper control of time intervals, signal output levels, and multiplex combinations.

Referring to FIG. 2, in the next time interval Dt2, the signal output V0 is applied to the electrodes E1 and E3, as illustrated by the + and the − symbol beneath their respective temperature readout histograms. Electrode E1 has the + output connected to it, and its temperature reading continues to rise above the value achieved in interval Dt1. The temperature reading at electrode E3 also begins to rise during Dt2. The temperature of E2, which during Dt2 does not have any connection to the signal output, remains about as it left of during interval Dt1, or may drop off slightly during Dt2 as the temperature of the tissue near it diffuses away. The exact temperature at each electrode during interval Dt2 or any other interval, depend on the tissue impedance and other tissue characteristics near each electrode, the amount of signal output applied (for example, in the illustration, the voltage V(RF)), the combination of electrodes that are switched in during the interval, the duration of the interval Dt2, and the thermodynamics of thermal diffusion that tends to wash away the tissue heat as time progresses by the thermal diffusion equation. These compound factors are at work during each heating interval and for each of the chosen multiplex combinations of electrodes that are switched in by the controller 2027 during that interval. The controller monitors the temperatures of each electrode during each interval, and the control algorithm in 2027 determines which combination of multiplexed electrodes should be switched in the next time interval and for how long so that the temperature distribution on all of the electrodes converges to the desires distribution according to clinical objectives. In one example, that objective can be to achieve a uniform temperature distribution on all of the electrodes as measured by their thermal readouts T1, T2, etc so that a desired and controlled therapeutic lesion volume of target tissue is achieved.

Referring to FIG. 2, in the next time interval Dt3, the + output is applied to E2 and the − output is applied to E3. The controller 2027 controls this because it detects that the temperatures of E2 and E3 lag that of E1 so that power must be delivered to E2 and E3 to increase their temperatures. The temperatures of E2 and E3 then rise. The temperature of E1 begins to fall lower by thermal diffusion. At an appropriate duration determined by the degree of rise of the temperatures of E2 and E3 the interval Dt3 is ended, and the next interval Dt4 is started. In the interval Dt4, the controller 2027 switches the + output to E1 and the − output is applied to both E2 and E3. This is done because the temperature of E1 must be increased more rapidly without excessive increase of the individual temperatures on E2 and E3, so that by using both E2 and E3 as the other − bipolar component, the power is shared by both them. In the interval Dt5, E1 and E2 are used as one side of the bipolar pair, and E3 alone is the other side of the bipolar pair. This results in E3 heating up preferentially, and the temperatures of the three electrodes becomes equal. If the temperature levels and durations are satisfactory, the process can be stopped. If the temperatures are too low, a further series of intervals like Dt1 through Dt5 can be continued and the output V(RF) can be incrementally increased. One additional type of interval (not depicted in FIG. 2) can also be used in which the + output is applied to E2 and the − output is applied to both E1 and E3. At the end of each interval the controller algorithm determines how long the interval lasts and which multiplex bipolar combination will be activated in the succeeding interval. A continuing series of such cycles can ultimately achieve the desired temperature on the electrodes, and procedure can be terminated.

The duration, number, combinations of multiplexing pairs and temperature objectives for each interval, the level incremental changes in the signal output level applied to the electrodes during each interval, and the ultimate end point temperature distribution on the electrodes for the desires clinical objectives can be determined and adjusted by the controller 2027 based on the measured temperatures at each point in time during the heating process, the degree and rapidity of the temperature rise at each interval, the electrodes' impedances and temperature imbalances at each point in time, and the controller's control algorithm that utilizes this information to determine the successive switching combinations on the multiplexing process.

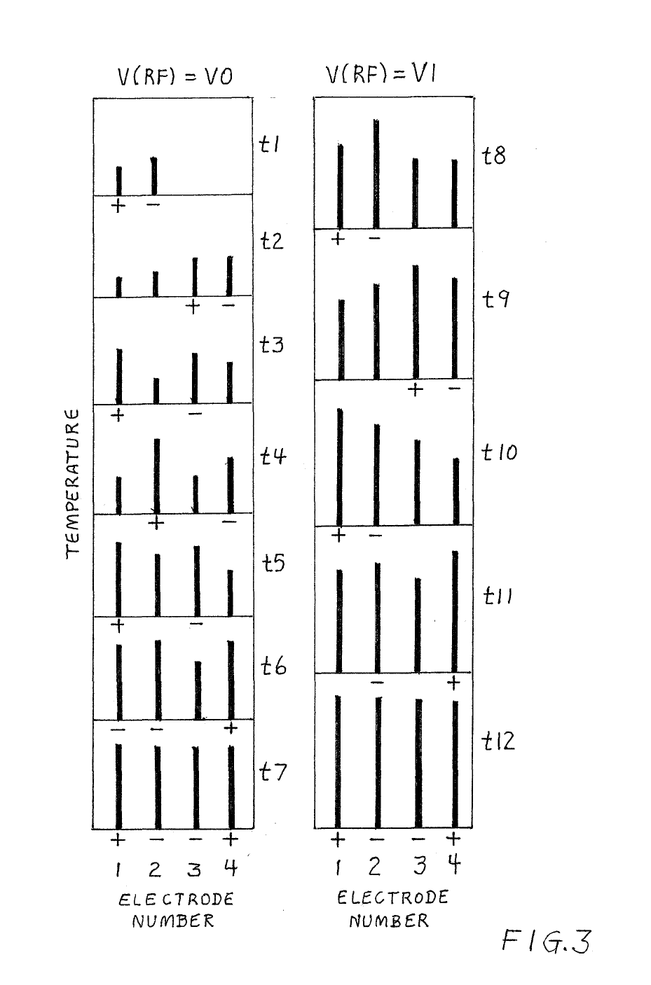

Referring to FIG. 3, a schematic example of multiplexing bipolar lesioning is shown for N=4 electrodes. In this example, n refers to the number of electrodes E1, E2, E3, E4 connected to one pole of the output of a generator 2011, and m refers the number of electrodes E1, E2, E3, E4 connected to the other output of that generator 2011. For example, n can refer to the number of electrodes connected to the + output and m can refer to the number of electrodes connected to the − output. The example comprises use of intermixed switching of bipolar pair combinations including switching on individual bipolar pairs of electrodes for which n=1 and m=1; switching on bipolar pairs in which n=1 and m=2; and switching on bipolar pairs in which n=2 and m=2. Said intermixed switching is done by the controller 2017 to achieve a balance of temperatures on all the electrodes. Furthermore, the example illustrates the increase of the output signal during the multiplexing sequences to raise the overall temperature distribution the electrodes. In the first switching intervals t1, t2, t3, t4, and t5, the + and the − outputs of the generator 2011 are applied to different pairs on individual bipolar pairs of individual electrodes, i.e., n=1 and m=1. At the end of t5, there are still differences in the temperatures at the four electrodes. The controller then switches to an n=1 and m=2 multiplex combination with the + output on the E4 electrode on one side, and the − output on the E1 and E2 electrodes on the other side. This increases the temperature of E4 and also, to a lesser degree E2. The controller 2027 switches to an n=2 and m=2 multiplex combination with the + output on the E1 and E2 electrodes and the − output on the E2 and E3 electrodes. This brings all the electrode temperatures up to an equal value. The above multiplexes for those time intervals are for a constant output signal voltage of V(RF)=V0. The temperatures for the four electrodes are still not high enough for the desired clinical end point, so further multiplex sequences are continued but at a higher voltage setting. These are illustrated by the intervals t8 through t12 which are done at V(RF)=V1, where V1 is greater than V0. The intervals t8 though t11 involve bipolar pairs with single electrodes on each pole; i.e., n=1 and m=1. Then for interval t12, the controller switches to an n=2 and m=2 multiplex combination to bring the temperatures of the four electrodes to an equal value. This value has been increased over the value at the end of the previous V(RF)=V0 series of intervals. If the achieved temperatures value is clinically sufficient, the procedure can be stopped. However, if it is clinically necessary to achieve a higher temperature, then further multiplex sequences can be applied with higher voltages levels V(RF). In another example, time intervals with n=1 and m=3 can also be used, although they are not depicted in the particular example shown in FIG. 3.

The time intervals can have a range of values according to clinical objectives and the controller algorithm. In one example, the intervals, such as Dt1 through Dt5 in FIG. 2 or t1 through t12 in FIG. 3 can be in the range of milliseconds, or seconds, or minutes, or tens of minutes, or longer depending on the tissue conditions near the electrodes, the rapidity of the desired temperature rise, the number of electrodes N, the characteristics of electrical conditions of the tissue, such as impedance, around the electrodes, and the incremental amount of temperature raise desired in a given interval or series or intervals, and the end temperature that is desired. In one example, the controller 2027 can cycle around the multiplexed bipolar pairs quickly and repetitively. The dwell time of an interval in which any multiplexed bipolar pair is connected to the signal output can be in the range of: one microsecond to one millisecond; or one millisecond to one second; or one second to one minute; or one minute to tens of minutes or even hours. The duration of the interval and cycle times can depend, for example, on the degree of smoothness of temperature changes desired during the interval. It can also depend on the desired speed of feedback of temperature readings from readout 2024 to controller 2027 to achieve a desired feedback and control performance. For example, interval times in the range of one millisecond to several seconds can have the advantage of fast feedback and rapid convergence of the temperature trends on the electrodes toward a desired distribution of temperature on the electrodes. In another example, the interval can be in the range of tens of milliseconds, hundreds of milliseconds, or seconds, or minutes in the case in one example, that the electrodes impedances and conductive tip sizes are relatively uniform and there is no runaway of temperature on any one or more of the electrodes during the RF heating. In one example, this can be dependent of the uniformity of tissue characteristics around the N inserted electrodes, or the rapidity of the desired temperature rise and convergence to a desired temperature distribution according to clinical needs. The interval times can also be of different durations. This can depend on the degree of heating of any given bipolar electrode pair during the interval or the criterion of the controller algorithm to converge to a desires temperature distribution during the interval.

One advantage of the examples of FIG. 2 and FIG. 3 is that because the multiplex bipolar switching is done only between the inserted electrodes, a temperature distribution objective for the electrodes (which in these examples is that of achieving a uniform temperature of a desired level) can be accomplished without the need for a ground pad GP placed on the patient's skin. This saves time and money for the clinician, and it avoids complications of cabling that are involved when a ground pad is used.

Another advantage of the examples of FIGS. 1, 2, and 3 is that by using multiplexed bipolar electrodes configurations, the current from the electrodes, and therefore the heating of the tissue around the electrodes, can progress between the electrodes so that more effective filling of the heat lesion between the electrodes is accomplished. This is important in clinical situations where neural, tumor, or other target structures are spread out in the target volume and it is desirable to fill in the interstices and spaces between the implanted electrodes so as to more effectively cover the target volume.

Another advantage of the multiplexed bipolar system and method herein is that if one electrode of the pair of the bipolar electrodes configuration does not heat up compared to the other electrode of the bipolar pair, then by switching in another multiplexed electrode bipolar configuration, as illustrated by the exemplary embodiment s of the FIGS. 1, 2, and 3, the heating of the lagging electrode (or electrodes) can be brought up preferentially so that the objective of a more uniform thermal distribution among all of the electrodes can be approached.

Referring to FIG. 2 and FIG. 3, in another example, the number of electrodes N can be any integer number greater than or equal to three, and the n and m can each take on any integer value greater than or equal to 1 such that N≧n+m.

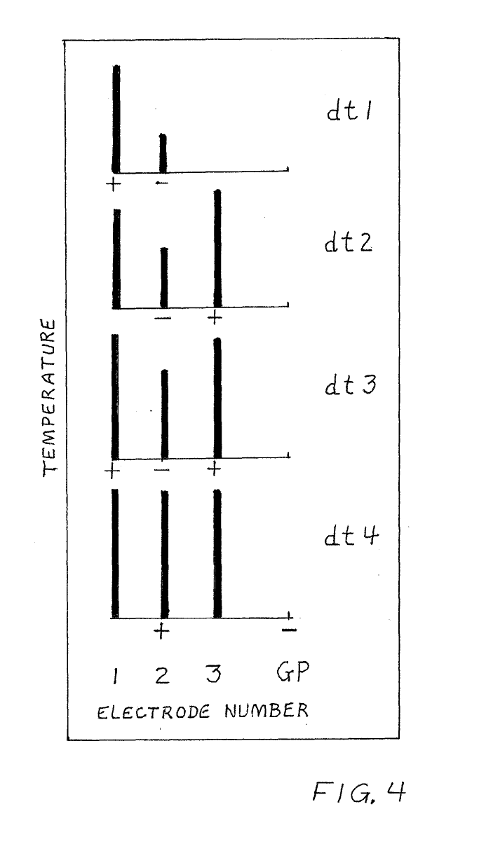

Referring to FIG. 4, an example of a heating sequence is schematically shown comprising intervals in multiplexing bipolar switching is done, in one series of intervals, among N=3 inserted electrodes and, in another interval, the bipolar switching is done between inserted electrodes and a ground pad GP. In one example, the generator signal output during interval dt1 is applied across E1 and E2 as schematically indicated be the + and − symbols beneath the corresponding electrodes positions on the temperature versus electrode number histogram. Because of tissue impedance differences or other inhomogenieties of the tissue or electrode characteristics, in this example, E1 heats up much faster that E2 during dt1. The controller 2027 determines the next interval dt2 will involve a bipolar connection between E2 and E3. During dt2, E3 heats up faster than E2, so that E2 still lags in temperature behind E1 and E3. Next the controller, having detected these temperatures at the end of dt2, switches to a multiplexed bipolar connection comprising the − pole connected to E2 and the + pole connected to both E1 and E3. Under some conditions of tissue impedances near the electrodes that would be a good algorithmic step for the controller 2027 to take because, in the example, the current from E1 and form E3 will both flow to E2. Also, the thermal diffusion of the heat from E1 and E3 will also summate towards E2. Therefore, under some conditions the controller algorithm would predict that E2 could heat up preferentially, and thus its temperature would tend to catch up with higher temperatures of E1 and E3. However, in this illustrative example in FIG. 4, the tissue and electrodes characteristics around the three electrodes are such that E2's temperature still does not catch up with temperature s of E1 and E3 as shown in the diagram for interval dt3. In this example, a ground pad (reference electrode) GP, which is in contact with the patient's skin, as illustrated in FIG. 1, and the controller makes the decision step to switch the signal output of generator 2011 between the inserted electrode E2 and the skin-based electrode GP. As is commonly known about ground pads, typically their surface area is sufficiently larger that the inserted electrodes conductive tip area. So there is no substantially heating of the tissue near the ground pad GP. Therefore the temperature of GP does not rise during the interval dt4. However, all of the generator's output current is now running from E2 to the GP electrode, and the temperature of the tissue at E2 rises as shown in FIG. 2 Thus the temperature of E2 can catch up to the temperatures of E1 and E3, and the temperature distribution of all of the electrodes becomes balanced and substantially equal.

One advantage of the embodiment of FIG. 4, is that, in the situation of inhomogeneous tissue impedances or other electrode characteristics of the inserted electrodes, and in which one of the electrode temperatures is difficult to bring up to the temperatures of the other electrodes, it is convenient to connect as one side of the bipolar pair to a ground pad or surface area electrode to boost the heating on one or more of the inserted electrodes during one or more of the time intervals. Thus the mixed multiplexing of bipolar connections comprising switching between only inserted electrodes and switching between inserted electrodes and a reference electrode can speed up the convergence of the temperatures of the inserted electrodes to a predetermined target temperature distribution.

One advantage of the embodiment presented in FIG. 4 is that the electrical output signal of the generator can flow among electrodes placed in the target volume or region of the body, as well as flowing from electrodes placed in the target volume of region of the body to a ground pad, during the same treatment period. Another advantage of the embodiment presented in FIG. 4 is the generator can dedicate a predominance of the signal output to be delivered between electrodes inserted in a target region, with a smaller amount of the signal output delivered between said electrodes inserted in the target regions and the ground pad; one objective of this said dedication can be focusing of currents and temperature in the region between said electrodes inserted in the target region and at the same time maintaining the temperatures measured at all said electrodes inserted in the target region.

Referring to FIG. 4, in another example, the number of inserted electrodes N can be any number greater than or equal to two. In another example, the ground pad GP can be an electrode inserted into a non-treatment region of the body, such as a large muscle that is remote of the treatment site.

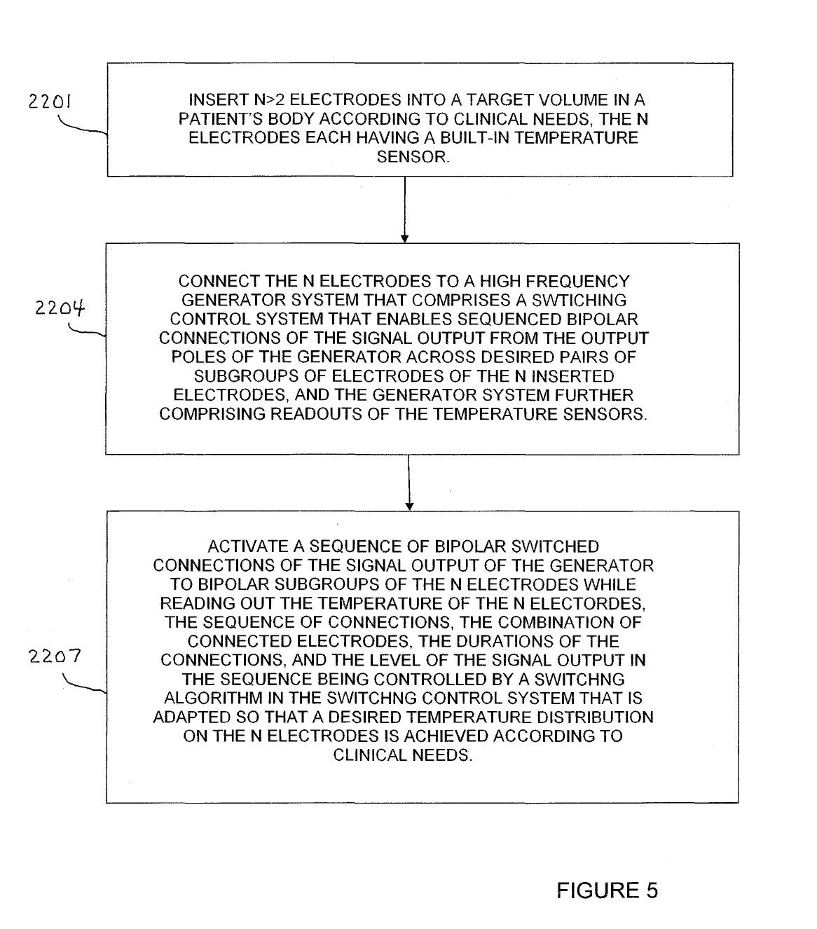

Referring to FIG. 5, a process is shown for activation of multiplexed bipolar electrodes that includes a step 2201 in which greater than two (N>2) electrodes are inserted into the patient's body so that the electrodes' active or conductive tips are in a target tissue volume in the body. In another example, step 2201 can include selecting a target tissue volume or target region beforehand base on imaging or other diagnostic analysis of the body. In one example, the electrodes can have built in temperature sensors to measure the temperature of the tissue near the electrode's tip. In another example, the electrodes can be cannulae that are inserted into the body, and temperature sensors can be probes that are inserted into the cannulae. In step 2204, the N electrodes are connected to a generator system that can comprise a high frequency generator and a switching control system as shown in FIG. 1. The step further comprises switching the signal output from the high frequency generator to combinations of subgroups of the N electrodes. Said subgroups can be designated as a group of n electrodes and another group of m electrodes such that the output connections of the generator is put across the n group and the m group so that the n group and the m group become a bipolar pair. The high frequency current from the generator can, in one example, flow between the n group and the m group to heat the tissue around and between these groups of electrodes. The switching control system can switch during successive time intervals between different n and m groups to control the heating of the tissue near the N electrodes. In step 2207, a sequence of switched patterns of connections between the n-group and the m-groups is activated. The signal output, the sequence of connections, the duration of the switched connections are controlled by the switching control system. The temperature at each of the N electrodes is measured and read out in the control system. The control system has a built-in algorithm that can analyze the temperatures and the successive heating level during the switching sequences, and can determine which n-group and m-group of electrodes are switched in for successive intervals. It can also determine the signal output levels for the each successive intervals and switched-in pairs of n-group and m-group electrodes to achieve a desired clinical objective. In one example, the objective can be that all of the electrodes achieve a desired temperature level. The control system can also determine when the entire process can be terminated based on clinical objectives.

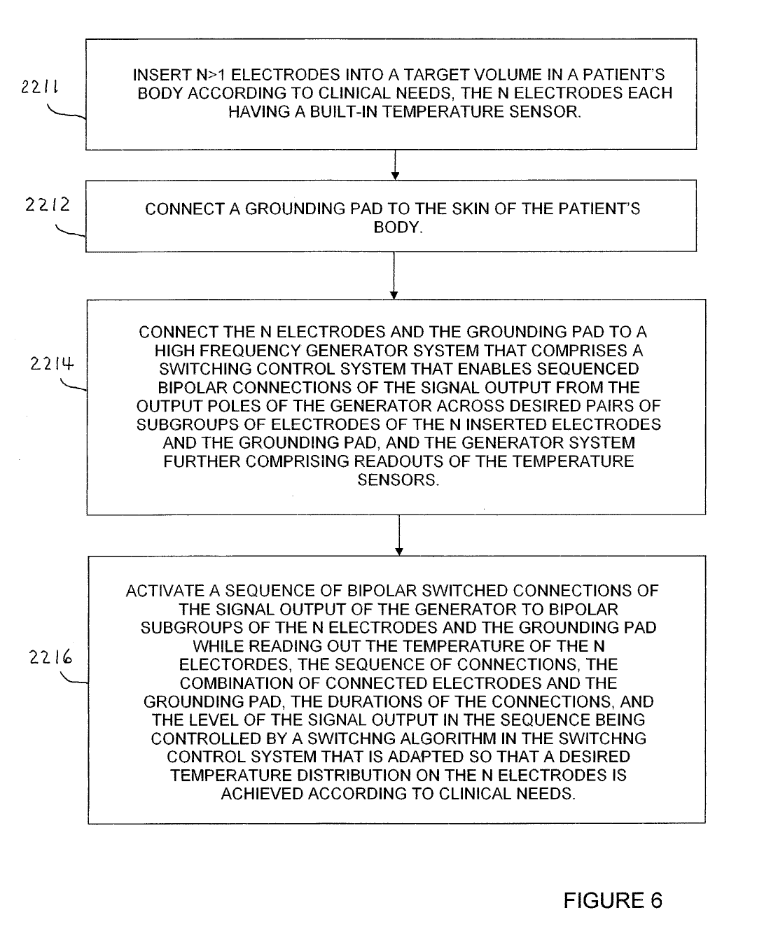

Referring to FIG. 6, a process is shown that includes step 2211 in which N>1 electrodes are inserted into the patient's body, and associated temperature sensors are connected and/or built into the electrodes to measure the temperature of the tissue near the electrodes. In step 2212 a ground pad electrode, or another type of reference electrode, is placed on the patient. In one example, the ground pad is a surface area electrode that is attached to the patient's skin. In step 2214, the N inserted electrodes and the grounding pad electrode are connected to a generator system that can comprise a high frequency generator and a switching control system as shown in FIG. 1. The step further comprises switching the signal output from the high frequency generator to combination of subgroups of the N electrodes and/or the grounding pad electrode, the subgroups of electrodes can be designated as a group of n electrodes, the n-group electrodes, and another group of m electrodes, the m-group electrodes, such that the output connections of the generator is put across the n group and the m group, or across the n-group electrodes and the grounding pad, or across a combination of the n-group, m-group, and/or the grounding pad electrode. The high frequency current from the generator can, in one example, flow between the n group and the m group, or across the n-group electrodes and the grounding pad, or across the n-group electrodes and a combination of the m-group electrodes and the grounding pad electrode to heat the tissue around and between these groups of n-group and m-group electrodes according to clinical objectives. The switching control system can switch during successive time intervals between different n-groups, m-groups, and the grounding pad to control the heating of the tissue near the N electrodes. In step 2214 a sequence of switched patterns of connections between the n-group, m-groups, and the grounding pad is activated. The signal outputs, the sequence of connections, and the duration of the switched connections are controlled by the switching control system. The temperatures at the N electrodes are measured and readout in the control system. The control system has a built-in algorithm that can analyze the temperatures and the successive heating level during the switching sequences, and determined which n-group, m-group, and grounding pad combinations of electrodes are switched in for successive intervals. It also determines the signal output levels for the each successive intervals and switched-in pairs of n-group, m-group and grounding pad electrodes to achieve a desired clinical objective. In one example, the objective can be that all of the N electrodes achieve a desired temperature level. The control system can also determine when the entire process can be terminated based on clinical objectives.

Referring to FIG. 6, in one example, the number of inserted electrodes can be N=2. This single bipolar pair, if heated alone, can have the problem of uneven temperature rise of the tissue near the two electrodes due to different tissue impedances of the nearly tissue. In that case, the controller can switch to using the ground pad and only the electrode with the lower temperature to give a boost in temperature to the electrode while giving no heating to the other electrode. This switching between a pure bipolar implanted pair to the electrode plus ground pad, can then equalize, on average the temperatures around each of the N=2 two electrodes. In another example, N can be three or more and, as illustrated in FIG. 4, switching intermittently to the ground pad at the same time as one of the inserted electrodes can enable equalizing the temperatures on all of the electrodes. This combination of implanted multiplex bipolar switching and switching to one or more implanted electrodes against a ground pad, has the advantage of producing thermal filling between the electrodes like in pure bipolar implanted switching and the advantage of boosting the temperature on one give electrode that is lagging in temperature rise.

Referring to FIGS. 2, 3, 4, 5, and 6, in another example, the measured parameters being controlled can quantities other than temperature of an electrode. In one example, one of the parameters under control can be the temperature of tissue adjacent to an electrode. In one example, one of the parameters under control can be the power output delivered through an electrode. In one example, one of the parameters under control can be the current output delivered through an electrode. In one example, one of the parameters under control can be the impedance between an electrode and other structures, such as other electrodes. In one example, one of the parameters under control can be the resistance between an electrode and other structures, such as other electrodes. In one example, one of the parameters under control can be a function of the electrical signal output connected to an electrode. In one example, one of the parameters under control can be the water content of tissue nearby an electrode. In one example, one of the parameters under control can be the blood perfusion of tissue in nearby an electrode. In one example, one of the parameters under control can be a physical quantity measured at that electrode. In one example, one of the parameters under control can be the time-dependence of one of the exemplary parameters just stated. In one example, one of the parameters under control can be a physical quantity measured in the tissue adjacent to or near to that that electrode. In one example, the same type of parameter can controlled for all electrodes. In one example, different types of parameters can be controlled across the electrodes.

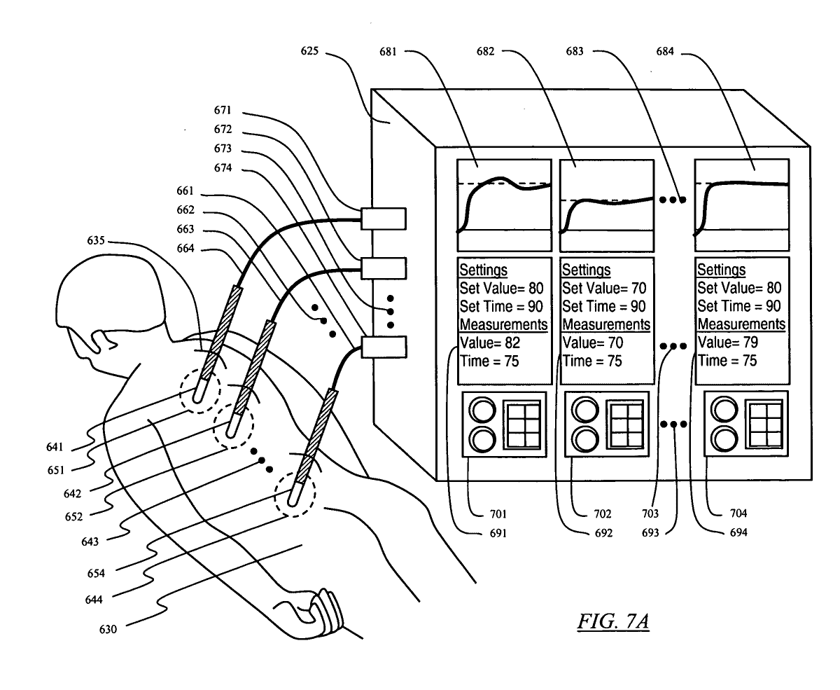

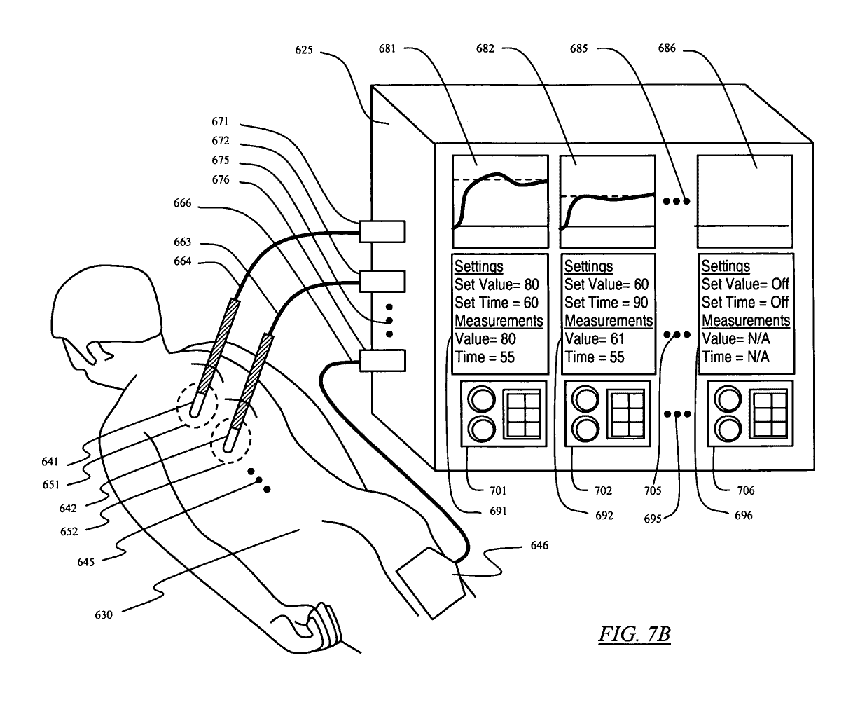

Referring to FIG. 7A and in accordance with one example of the present invention, a system for application of electrical energy to tissue is provided. A generator 625 has connection jacks, illustrated by four electrodes 671, 672, 673, 674, representing at least three electrodes, where dot 673 represents schematically possible additional electrode connection jacks. Electrodes 641, 642, 643, 644 are connected to electrode connection jacks 671, 672, 673, 674 by cable systems 661, 662, 663, 664, respective and where dot 663 can represent schematically possible additional cable systems, and where dots 643 represents schematically possible additional of electrodes (as was not depicted in the example provided in FIG. 1).

In one example, generator 625 can be an electrosurgical generator. In one example, generator 625 can be a radiofrequency generator. In one example, generator 625 can be a high-frequency generator. In one example, generator 625 can generator electrical output signals.

Each electrode, such as 641, 642, 643, or 644, can have an active region, for example, an exposed conductive tip, through which electrical output of the electrosurgical system 625 can be applied, and can have other regions which are composed of an electrical insulator. Said at least three electrodes 641, 642, 643, 644 can be placed in a living body 630, such as the human body. Said at least three electrodes 641, 642, 643, 644 can be placed in a living body 630 percutaneously. For example, electrode 641 is shown passing through the surface of the skin 635. Said at least three electrodes 641, 642, 643, 644 can also be placed in a living body 630 during an open surgical procedure, a laproscopic surgical procedure, or an endoscopic surgical procedure.

For example, each of electrodes 641, 642, and 644 can have an elongated shaft with an active conductive tip at its distal end, with the remaining proximal aspect of the shaft being non-active. An active zone can be constructed from an electrical conductor, such as a metal, which is in electrical communication with the electrical output of the generator 625 via wires shrouded in the insulated, non-active portions of the electrode probe and cable system.

Each electrode, such as 641, 642, 643, or 644, can have an active region through which electrical output of the electrosurgical system 625 can be applied, and can have other regions which are composed of an electrical insulator. For example, each of electrodes 641, 642, 643, and 644 can have an elongated shaft with an active region at its distal end, with the remaining proximal aspect of the shaft being non-active. An active zone can be constructed from an electrical conductor, such as a metal, which is in electrical communication with the electrical output of the generator 625 via wires shrouded in the insulated, non-active portions of the electrode probe and cable system.

The electrical output of the generator 625 can have a physical effect in a region of influence near an electrode's active zone. Electrodes 641, 642 and 644 have regions of influence 651, 652, and 654, respectively, in the tissue of living body 630. Ellipsis 643 can represent a non-negative integral number of electrodes, where each electrode has an active zone and an associated region of influence. The physical effect in an electrode's region of influence can be a change in temperature, change in electrical resistance, change in electrical impedance, protein denaturation, coagulation, tissue desiccation, tissue ablation, lesion generation, tumor ablation, destruction of nervous tissue, stimulation of nervous tissue, modification of nerves, exposure of tissue to electrical phenomena, integrated exposure of tissue to electromagnetic fields, exposure of tissue to an electric field, exposure of tissue to a current density field, frictional heating, tissue heating, ohmic power loss, and other physical effects that should be familiar to one skilled in the art. The regions of influence of different electrodes can overlap or can be physically disjoint. The extent of the region of influence for a given electrode can be influenced by another electrode. The region of influence of two electrodes can be between those electrodes, such as when two electrodes are connected to signal output of the generator 625 in a bipolar manner.

The placement of treatment electrodes 641, 642, 643, 644 can be configured so that their regions of influence are near to, next to, adjacent to, inside, or enveloping a target structure, in whole or in part. A target structure can be a nerve; nervous tissue; a set of nerves; a tumor; an organ; a tissue structure; an anatomical structure; a membrane; a blood vessel; a set of blood vessels; tissue surrounding a tumor; tissue adjacent to a tumor; tissue carrying blood supply for a tumor; a region of the liver; a region of the heart; a region of the kidney; a region of the brain; a region of the lung; a region of the pancreas; a region of the prostate grand; a region of the breast; tissue in the sacroiliac region; innervations of the sacroiliac joint; the medial branch nerves; innervations of a facet joint; medical branch nerves in the lumbar, thoracic, or cervical region of the spine; intraarticular nerves; a joint; and the interior of a joint.

It is understood that said electrodes, cables, and jacks can be integrated into one or more physical structure. For example, a single electrode connection jack can connect to a branching cable which can connect to multiple individual electrodes. For example an electrode connection jack can refer to one or more pins in a larger connection structure. For example, multiple electrodes can be integrated into a single physical structure. For example, multiple active zones can be integrated in a single physical structure such that each zone is substantially electrically isolated from the other active zones, and such that said active zone are connected to the electrosurgical generator 625 in a manner that they each act as an individual electrode 641, 642, 643, 644. Each electrode, such as 641, can take a variety of forms which should be familiar to one skilled in the arts of electrosurgery, tissue ablation, neural tissue ablation, or tumor ablation. For example, each electrode, such as 641, 642, 643, or 644, can be a combination of a cannula and electrode used for radiofrequency tissue ablation, such as the Cosman CC cannula and Cosman CSK-TC electrode, respectively. For example, each electrode can be an internally-cooled radiofrequency electrode, such as the ValleyLab CoolTip electrode.

The electrical output of the electrosurgical system 625 can be electrical current, electrical voltage, electoral potential, electrical energy, electrical power, a reference signal, a reference potential, a radiofrequency signal, a stimulation signal, a pulsed radiofrequency signal, radiofrequency current, radiofrequency voltage, a signal whose carrier signal is in the range 300 kHz to 1000 kHz, a signal whose carrier signal is 480 kHz, a 50 kHz signal, a signal configured for impedance monitoring, an oscillating signal, a signal configured to ablate tissue, a signal configured to coagulate blood, a signal configured to stimulate nerve tissue, or other types of outputs which should be familiar to one skilled in the art of electrosurgery, tumor ablation, radiofrequency lesioning, and radiofrequency pain management. The electrical output of the electrosurgical system can be the superposition of multiple signal types, such as the aforementioned types.

Electrodes 641, 642, 643, or 644 can be connected and disconnected from the output of the electrosurgical system 625 by means of switches integrated into the electrosurgical system 625, integrated into the cable system 661, 662, 663, 664, or otherwise connected to both the electrosurgical system and the electrodes. The switching system can be manually controlled, automatically controlled, or both. The switches can connect each electrode 641, 642, 643, or 644 to multiple different system electrical potentials, either at the same time, or at different times. The switches can connect each electrode 641, 642, 643, or 644 to multiple different output types, either at the same time, or at different times. For example, switches can be configured such that, by changing the state of the switches, each electrode can be connected to a reference potential, connected to a radiofrequency signal, or disconnected from system potentials. An electrode that is disconnected from direct connection to system power supplies by its switches can be referred to a “floating” or “electrically passive”. The electrical state of a floating electrode can be influenced by the electrosurgical system via output delivered to other electrodes, but a floating electrode does not draw or emit a substantial amount of electrical current from system power supplies through its cable system. Switches can be configured such that pairs of electrodes can be energized in a bipolar manner, where each electrode in a pair serves as the path for return currents from the other electrode in the pair. Switches can be configured such that groups of electrodes are connected to various system potentials at the same time. Switches can be changed over time such that at any time only one pair of electrodes is connected to electrical signal output in a bipolar manner, with all other electrodes floating. Switches can be changed over time such that groups of electrodes are energized in sequence, such that when electrodes in a group are connected to system potentials, electrodes not in that group are floating. Switch positions can be changed at a rate sufficient to achieve system control objectives. In one example, switch positions can be changed after durations of at most 1 millisecond. In one example, switch positions can be changed after durations of at most 2 milliseconds. In one example, switch positions can be changed after durations of at most 5 milliseconds. In one example, switch positions can be changed after durations of at most 10 milliseconds. In one example, switch positions can be changed after durations of at most 50 milliseconds. In one example, switch positions can be changed after durations of at most 100 milliseconds. In one example, switch positions can be changed after durations of at most 500 milliseconds. In one example, switch positions can be changed after durations of at most 1 second. In one example, switch positions can be changed after durations of at most 2 seconds. In one example, switch positions can be changed after durations of at most 3 seconds. In one example, switch positions can be changed after durations of at most 5 seconds. In one example, switch positions can be changed after durations of at most 10 seconds. In one example, switch positions can be changed after durations of at most 15 seconds. In one example, switch positions can be changed after durations of at most 30 seconds. In one example, switch positions can be changed after durations that are configured to achieve a control objective. In one example, switch positions can be changed after durations that are configured to achieve a clinical objective. One advantage of selecting the a particular maximum duration in which switch positions are changed is that the said duration can be configured to physical dynamics of a parameter under control.

Electrodes 641, 642, 643, or 644 can also be connected and disconnected from the output of the electrosurgical system 625 by means of enabling and disabling power supplies associated with the electrosurgical system 625. For example, an electrode can be connected to only one power supply, and that electrode can be put into an electrically passive “floating” state by disabling its sole dedicated power supply.

The electrosurgical generator 625 can have a measurement system. The measurement system can monitor the state of the three or more electrodes 641, 642, 643, and 644, and can measure time. The measurement system can monitor the electrical output delivered to each electrode, such as the Voltage, Current, or Power delivered to an electrode. The measurement system can measure over time tissue properties related to the electrical output of the generator, such as the tissue resistance and tissue impedance. The measurement system can monitor signals from sensors integrated into any electrode. For example, each electrode can include a temperature sensor. For example, each electrode can include sensors configured to monitor its active zone or its region of influence in the tissue. For example, each electrode can include a temperature sensor in the active zone configured such that the temperature sensor's readings are indicative of the tissue temperature in that active zone's region of influence in the tissue. The measurement system can associate each measurement it collects with a time stamp. The measurement system can compute functions of some measurements to produce other measurements. A measurement can be referred to as a “parameter”. A measurement can be an estimate of a quantity based on other measured values. For example, the measurement system can divide a Voltage measurement V by a Current measurement I, to yield an Impedance measurement Z=V/I. For example, the measurement system can integrate or average functions of measurements over time. For example, the measurement system can measure the average power delivered to a particular electrode over a period of time. The measurement system can also monitor quantities that are not specific to only one electrode. For example, the measurement system can monitor the electrical potential between two electrodes. For example, the measurement system can monitor the total power delivered to a group of electrodes that are connected to system output potentials at the same time. Examples of an electrode-specific measurement, a function of electrode-specific measurements, an electrode-specific parameter, or an estimate of an electrode-specific quantity which an electrosurgical system 625 can control include, but are not limited to, a parameter that is influenced by the application of signal output to an electrode, the Voltage or RMS Voltage applied to an electrode, the Voltage amplitude of an radiofrequency signal applied to an electrode, the Current or RMS Current applied to an electrode, the Current amplitude of a radiofrequency signal applied to an electrode, the Power or average Power applied to an electrode, the energy applied to an electrode, the energy applied to an electrode over a period of time, the ohmic power loss in tissue to which an electrode is delivering energy, the Impedance measured at an electrode, the resistance measured at an electrode, the Impedance magnitude measured at an electrode, the Impedance phase measured at an electrode, the Temperature measured at or near an electrode, the electric field exposure of tissue near an electrode, the current density exposure of tissue near an electrode, the power-density exposure of a tissue near an electrode, and the average exposure of tissue near an electrode to electromagnetic phenomena.

The electrosurgical generator 625 can have alphanumerical displays 691, 692, 693, and 694 that display one or more measured values for each electrode 641, 642, 643, and 644, respectively, where ellipsis 693 represents a non-negative, integral number of displays that correspond respectively to the non-negative, integral number of electrodes represented by ellipsis 643. Each electrode display 691, 692, 693, and 694 can display a timer value. The electrosurgical generator 625 can have graphical displays 681, 682, 683, and 684 that graph over time one or more measured values for each electrode 641, 642, 643, and 644, respectively, where ellipsis 683 represents a non-negative, integral number of displays that correspond respectively to the non-negative, integral number of electrodes represented by ellipsis 643. Said graphical displays can be dynamic plots which are updated regularly to show ongoing changes.

The electrosurgical generator 625 can have a control system. The control system can use measurements from the three or more electrode 641, 642, 643, and 644 to adjust the output delivered to each electrode for the purpose of achieving a control objective for each electrode. In one example, the control objective for each electrode is unique. In another example, the control objective for each electrode is substantially independent of the control objectives associated with other electrodes. In another example, the number of control objectives can exceed the number of electrodes. A control objective can be that of changing the value of a measurement, or a function of a measurement or measurements, from its idle value. A control objective can be that of holding the value of a measurement near a target value, within a target range, within a range of a target value, or within a range of a time-varying target value. A control objective can be configured to achieve, approach, or control a physical effect, such as the aforementioned physical effects in the region of influence of an electrode. A control objective can be the control of an electrode-specific measurement, an electrode-specific parameter, or an estimated electrode-specific quantity. For example, a control objective can be that of holding a temperature measured at an electrode at a set value. In one example, a control objective can be that of holding the temperature measured at an electrode within 0.5° C. of a desired value. In one example, a control objective can be that of holding the temperature measured at an electrode within 1° C. of a desired value. In one example, a control objective can be that of holding the temperature measured at an electrode within 2° C. of a desired value. In one example, a control objective can be that of holding the temperature measured at an electrode within 5° C. of a desired value. In one example, a control objective can be that of holding the temperature measured at an electrode within 10° C. of a desired value. In one example, a control objective can be that of holding the temperature measured at an electrode within 20° C. of a desired value. In one example, a control objective can be that of holding the temperature measured at an electrode within a desired range of temperature values that is configured to achieve a clinical objective such as achieving cell death. One advantage of selecting a small range around a set value is that a more predictable lesion can be formed. One advantage of selecting a larger range around a set value is that a controller is less constrained in achieving its objectives. For example, an electrode-specific control objective can be that of holding the temperature measured at an electrode below a value, such as 42° C., or such as 45° C. For example, a control objective can be that of holding the temperature measured at an electrode above a value, such as 42° C., 45° C., 50° C., 60° C., 70° C., 80° C., 90° C., 95° C., 100° C., or a value configured to achieve a clinical objective. For example, a control objective can be that of delivering an amount of electrical power to an electrode. For example, a control objective can be that of delivering an amount of electrical current to an electrode. For example, a control objective can be that of delivering an amount of electrical voltage to an electrode. For example, a control objective can be that of delivering an average power to an electrode over a time period, such as a time period less than 100 millisecond, a time period less than 1 second, a time period less than 15 seconds, a time period less than 30 seconds, a time period less than 1 minute, a time period dependent on measured values, a time period terminated by a measured rise a measured impedance, a time period terminated by a change in a measured electrical current, the a time period terminated by a measured rise in temperature. For example, a control objective can be that of holding the impedance between a specific electrode and reference structures at, near, or below a set value; an example of said reference structure can be one or more other electrodes. For example, a control objective can be that of preventing a rise in the impedance between an electrode and reference structures, where said impedance rise is indicative of a change in the temperature of tissue near the said electrode. For example, a control objective can be that of preventing a rise in the impedance between an electrode and reference structures, where said impedance rise is indicative of tissue boiling or beginning to boil near said electrode.

In one example, a parameter can be said to be “under control” or “controlled” if it induced to fall within a target range of values. In one example, said target range can vary over time. In another example, said target range can be fixed. In one example, a parameter that has been induced into a target range by delivery of electrical energy by one or more electrodes, can remain in said target range for a period of time after the cession of all energy delivery that substantially affects said parameter. In one example, the temperature of an electrode placed in tissue can stay within a target temperature range a period of time after energy delivered through that electrode has ceased due the thermal dynamics of the said electrode and tissue, even if energy delivered through other electrodes during said period of time does not substantially influence the temperature of the first said electrode. In one example, the power delivered to an electrode in bursts of energy can fluctuate, but still achieve an average power value that falls within a target range over a specified moving-average time window; in this example, the power can be said to be controlled. In one example, the average power delivered to an electrode in bursts of energy can fluctuate, but still fall within a target range over a moving time window; in this example, the power can be said to be under control. In one example, for an electrode whose temperature is elevated into a target range by bursts of electrical energy delivered to said electrode, the said temperature does not fall out of said target range between bursts of energy, if the rate of energy bursts rapid relative to the physical dynamics of said temperature; in this example, said temperature can be said to be controlled. In one example, for the tissue near an electrode whose impedance has a value induced into a target range by bursts of electrical energy delivered to said electrode, the said impedance does not leave that target range between bursts of energy, when the rate of energy bursts rapid relative to the physical dynamics of said temperature; in this example, said impedance can be said to be controlled.

The electrosurgical generator 625 can have control panels 701, 702, 703, and 704 by means of which a human operator can adjust the control objective and output parameters for each electrode 641, 642, 643, and 644, respectively, where ellipsis 703 represents a non-negative, integral number of controls that correspond respectively to a non-negative, integral number of electrodes represented by ellipsis 643. Each control panel can consist of one or more controls. For example, a control can be a lesion time, a set value for a measurement or a function of measurements, a target value for a measurement or function of measurements, a target range for a measurement or function of measurements, or a limit on the output level. A control panel can include a control knob, buttons, elements of graphical user interface, or items displayed on a touch screen. The control panels 701, 702, 703, and 704 can also be configured such that one or more specific control applies to more than one electrode, or to all electrodes. For example, all the controls in a single control panel can apply either to all electrodes, or only to all electrodes selected as active on the control panel. The electrosurgical generator 625 can have alphanumerical displays 691, 692, 693, and 694 which show the control settings for each electrode 641, 642, 643, and 644, respectively, where ellipsis 693 represents a non-negative, integral number of controls that correspond respectively to a non-negative, integral number of electrodes represented by ellipsis 643. The electro surgical generator 625 can have graphical displays 681, 682, 683, and 684 which graph measurements over time relative to control targets for those measurements, for each electrode 641, 642, 643, and 644, respectively, where ellipsis 683 represents a non-negative, integral number of controls that correspond respectively to a non-negative, integral number of electrodes represented by ellipsis 643. In FIG. 7A, each graphical display 681, 682, 683, and 684 shows, as an example, a graph in which time is plotted on the horizontal axis, a measured parameter is plotted on the vertical axis, and three lines are plotted on these axes: (1) a solid, straight line which can represent that idle or initial value of a measured quantity that is associated with the electrode to which the display corresponds; (2) a dashed line which can represent the target value of the said measured parameter; (3) a heavier line which can represent a plot of recent measured values over time. In accordance with the present invention, and as illustrated by graphical displays 681, 682, 683, and 684, each parameter measured for each of the three or more electrodes 641, 642, 643, and 644 can be controlled at the same time by the electrosurgical system 625 such that it changes from its initial value to a value near or at a target value. In one example, the said dashed line can represent a constant target value. In another example, the said dashed line can represent a target value that changes over time. Each graphical display can take one of a variety of forms which should be familiar to one skilled in the art, including the form of a bar graph, a graph in which time is plotted on the vertical axis and the measurement value is plotted on the horizontal axis, or in which time is plotted on the horizontal axis and the measurement value is plotted on the vertical axis.

The control system for an electrosurgical system can employ one or more of a number of types of stopping criteria to determine when to stop delivering generator output to an electrode. For example, the control system can discontinue delivering output to an electrode if a parameter exceeds a maximum value, or if a parameter falls below a minimum value. Examples of said parameters that can be employed for the purpose of a stopping criteria include, but are not limited to, a duration of time for which an electrode is energized, the duration of time elapsed since an electrode was initially connected to a system output signal, the number of bursts of generator output delivered to an electrode, the number of burst of radiofrequency energy applied to an electrode, the time-integral of an electromagnetic quantity applied to an electrode, the time-integrated Voltage applied to an electrode, the time-integrated Current applied to an electrode, the time-integrated Power applied to an electrode, the time-integral of the electric-field applied to tissue near an electrode, the time-integral of the current-density field applied to tissue near an electrode, the time-integral of the power-density field applied to an electrode.

The control system of the electrosurgical generator 625 can operate such that it intermittently determines an output signal, such as an output level, and switch positions which are configured to achieve electrode-specific control objectives associated with each of the three or more electrodes 641, 642, 643, and 644. The system settings or sequence of system settings determined by the control system can be effected by the system power supplies and switching system until the next determination by the control system. The time interval between said determinations by the control system can be referred to as the “control period”, “control update period”, or “control update time”. A said determination of system output and switches by the control system can be referred to as a “control update”. For each electrode, the control system can use the history of a measurement and the current control objective, such as a target value, to determine an output signal or output level for the upcoming control period. An example of an output level is the average power delivered to an electrode over the upcoming control period. Another example of an output level is the voltage amplitude, current amplitude, squared voltage amplitude, squared current amplitude, or average power of a radiofrequency signal delivered to an electrode. An example of a parameter that can be subject to a control objective is the temperature measured by an electrode. Another example of a measurement and a parameter subject to a control objective is the impedance measured at an electrode with respect to reference structures. The control system can produce a control update for each electrode by means of algorithms or methods such as a proportional controller, a proportional-integral (PI) controller, a proportional-integral-derivative (PID) controller, or another control algorithm which should be familiar to one skilled in the art. The control system can implement that control update for each electrode by energizing groups of electrodes in sequence, such that during the “on-time” in which an electrode group is connected to an electrical output signal, the switching system connects system power supplies to electrodes that are included in the current group, and the switching system disconnects from system power supplies all electrodes that are not included in the current group. The controller can assign different electrical potentials to each electrode in a group. The term “duty-cycle period” can refer to the period of time over which the energy is delivered to electrode groups sequentially. A given electrode can be assigned to one or more electrode groups. The controller can restrict the electrode groups to pairs, so that during each pair's on-time the constituent electrodes are connected to system power supply output in a bipolar configuration. The controller can assign one of two electrical potentials to each electrode in a group, for all groups during a control period. The controller can assign electrical potentials to each electrode in a group such that the potential difference between any two electrodes in that group is either zero or a non-zero value, for all groups during a control period. The controller can predict the output level, such as the electrical current, for each electrode in an electrode group while that group is connected to a generator output signal; said predictions can be made using, for example, past measurements, exploratory measurements, and system identification techniques. The timing of cycling among electrode groups and the output signals applied to each electrode in each electrode group can be determined by the control system such that over the duty-cycle period, each electrode is delivered an output signal which is equivalent to that specified by that electrode's control update for the purpose of achieving an electrode-specific control objective for that electrode. For example, for the purpose of controlling an electrode's temperature or impedance, an average output level can be delivered to that electrode over a period time by applying output to that electrode intermittently over the said period of time. The said determination of timing of cycling among groups and the assignment of output signals to electrodes in each group can be made by the controller by solving a system of equations. For three or more treatment electrodes, the controller can assign output signals and an on-time for each group by solving a system of equations. For example, said system of equations can state that, for each electrode, the total exposure to the output signal for that electrode is the sum of products of the output level and the on-time for each electrode group in which that electrode is included. The duration of the duty-cycle period and the duration of the control period can each be determined by the controller as part of each overall control update, or can each be pre-selected as part of the controller's design. The duration and of the duty-cycle period and the duration of the control period can each be configured such that each electrode's electrode-specific control objective can be targeted or achieved even if the specified output level is not delivered to each electrode in a continuous time segment of the said upcoming period of time. The duration and of the duty-cycle period and the duration of the control period can each be configured such that each electrode's electrode-specific control objective can be targeted or achieved even if the output is delivered to electrodes by duty-cycling among electrode groups during the said duty-cycle period.

In one example, the electrosurgical system 625 can individually control the temperature measured by each of three or more electrode electrodes 641, 642, 643, and 644 placed in the human body by energizing electrode groups in sequence such that the generator output delivered to each electrode in the course of switching among electrode groups is configured to control that electrode's measured temperature, without the use of an additional electrode, such as a ground pad. In one example, the electrosurgical system 625 can individually control the tissue impedance in the region of influence of each of three or more electrode electrodes 641, 642, 643, and 644 placed in the human body by energizing electrode groups in sequence such that the generator output delivered to each electrode in the course of switching among electrode groups is configured to control the impedance measured by that electrode, without the use of an additional electrode, such as a ground pad. In one example, the electrosurgical system 625 can individually control the average power delivered to each of three or more electrode electrodes 641, 642, 643, and 644 placed in the human body by energizing electrode groups in sequence such that the generator output delivered to each electrode in the course of switching among electrode groups is configured to control the average power delivered to that electrode, without the use of an additional electrode, such as a ground pad. In one example, the electrosurgical system 625 can achieve an electrode-specific control objective for each of three or more electrode electrodes 641, 642, 643, and 644 placed in the human body by energizing electrode groups in sequence such that the generator output delivered to each electrode in the course of switching among electrode groups is configured to control parameters subject to that electrode-specific control objective, without the use of an additional electrode, such as a ground pad.

It is understood that controls 701, 702, 703, 704 can be used to manually disable one or more of the electrodes 641, 642, 643, 644 plugged into the electrode jacks 671, 672, 673, 674, respectively, while three or more other of the electrodes are connected to electrical signal output. Said disabling of an electrode can be performed by opening the switches which connect that electrode to system power supplies, or by disabling the power supplies that are dedicated to that electrode.

Referring to FIG. 7A and in accordance with one example of the present invention, subsets of electrodes 641, 642, 643, and 644 can be connected to electrical output energy in a sequence, said sequence containing at least one step in which more than two electrodes are energized at the same time. In one more specific example, the number of control objectives can be less than the number of electrodes 641, 642, 643, and 644. In one more specific example, the number of controlled parameters can be less than the total number of electrodes connected to electrical output signals over the entirety of the sequence of steps. One advantage of these examples of the present invention is that a sequence that contains a step in which more than two electrodes are energized is more flexible than is a sequence that only contains steps in each of which at most two electrodes are energized. In one example, an electrode can be said to be “energized” if it connected to a output signal. In one example, an electrode can be said to be “energized” if it is connected electrical output current flows through said electrode.

Referring now to FIG. 7B and in accordance with one example of the present invention, the electrosurgical system 625 can also be operated in a configuration that includes an indifferent electrode 646, such as a ground pad, reference electrode, or other type of electrode for which the system 625 does not specify an individual control objective. A non-negative, integral number of treatment electrodes 641, 642, 645 can be placed in a living body 630, either through the skin 635 or in an open procedure. Said treatment electrodes 641, 642 are connected to jacks 671, 672 via cable systems 661, 662, respectively. Said treatment electrodes 641, 642 can each have a region of influence 651, 652, respectively, in the tissue into which they are placed. Ellipsis 645 can represent a non-negative, integral number of electrodes, each with a region of influence in the tissue in which they are placed, and each with a cable system that can connect to the jacks represented by ellipsis 675. The electrode connection jack 676 can be connected to an indifferent electrode 646, which can take the form of a ground pad, a ground plate, a dispersive electrode, an tissue-piecing needle, a non-tissue piecing elongated structure, and which can be placed on the surface of the skin, into the skin, under the skin, into a muscle, into a region of living body 630 not substantially related to, or substantially affected by, the electrical current carrier by said indifferent reference electrode 646. The indifferent electrode connection jack 646 can be the same electrode connection jack as 644 in FIG. 7A, or it can be a different specialized jack. Control panels 691, 692, 695, 696 can contain user controls for electrodes 641, 642, 645, 646, respectively, where ellipsis 695 represents a non-negative, integral number of control panels that correspond to electrodes 645. Alphanumeric displays 701, 702, 705, 706 can contain measurements and settings parameters for electrodes 641, 642, 645, 646, respectively, where ellipsis 705 represents a non-negative, integral number of alphanumeric displays that correspond to electrodes 645. Graphical displays 681, 682, 685 can graph measurement values for electrodes 641, 642, 645, respectively, where ellipsis 705 represents a non-negative, integral number of alphanumeric displays that correspond to electrodes 685. The settings of control panel 696 designate electrode 646 as a indifferent electrode, which can carry some of the return currents from the other electrodes 641, 642, 645. An alphanumeric display corresponding to the indifferent electrode 646 can indicate that the electrode is indifferent. The control system can have no specified control objective for the indifferent electrode 646. The control system can have cut-off controls for the indifferent electrode. For example, a cut-off control can be configured to stop generator output if a measurement related to the indifferent electrode exceeds or falls below a threshold value, even if the measurement is not specifically controlled. For example, a cut-off control can limit the output of the generator to treatment electrodes 641, 642, and 645 such that their respective control objectives may not be achievable. The indifferent electrode can or cannot have a measurement system. The graphical display 686 and measurement display in alphanumeric display 706 for indifferent electrode 646 can be absent, can display that no measurement is being made for electrode 646, or can display a measured value that is not explicitly controlled by the electrosurgical generator. The control panel 696 can be absent and electrode jack 676 can be a fixed indifferent electrode jack. The controller of the electrosurgical system 625 can include the indifferent electrode 646 in some of the electrode groups connected to electrical output signals during phases of duty-cycle period. The controller of the electrosurgical system can implement duty-cycle periods in which there is an electrode group which does not contain the indifferent electrode 646, so that signal output flows between two or more of the treatment electrodes 641, 642, 645. For embodiments in which 641, 642, 645 represents two or more electrodes, the controller can include the indifferent electrode 646 for the purpose of achieving control objectives on all of the treatment electrodes 641, 642, 645. An advantage of the embodiment of the present invention provided in FIG. 7B, this that an electrosurgical system can deliver signal output both among treatment electrode 641, 642, 645, and between treatment electrodes 641, 642, 645 and the indifferent electrode 646, during the same operational session. An advantage of the embodiment of the present invention provided in FIG. 7B, this that an electrosurgical system can increase the range of operating conditions under which the control objectives of all treatment electrodes 641, 642, 645 can be achieved at the same time.

Referring to FIG. 7A and FIG. 7B and in accordance with one example of the present invention, it is understood that measurements, parameters, and quantities that are the subject of control objectives can be collected by devices that are physically separate from the electrodes shown in FIG. 7A and FIG. 7B. For example, temperature probes can be placed in or in contact with the body 630 and connected to the electrosurgical system

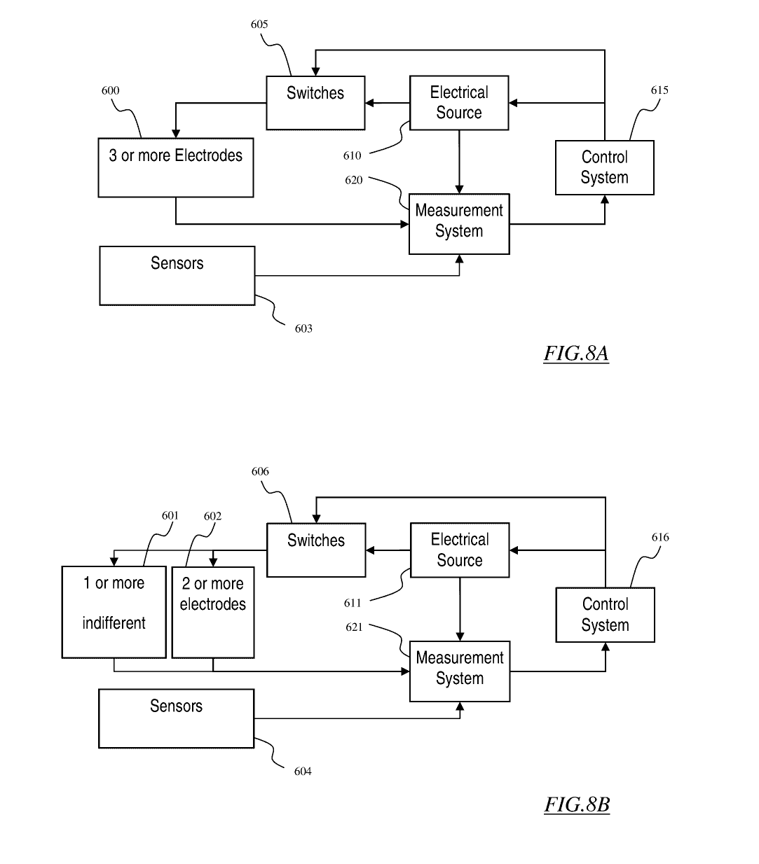

Referring now to FIG. 8A and in accordance with one example of the present invention, an electrosurgical system is provided. Said electrosurgical system can consist of three or more electrodes placed in a body of tissue 600; a switching system 605 that can connect and disconnect said electrodes 600 to electrical power supplies 610; one or more electrical power supplies 610 that can generate electrical output signals such as differences in electrical potentials; sensors 603 configured to monitoring the electrodes 600 or the tissue in which the electrodes 600 are in contact; a measurement system 620 which can monitor the electrical output of the generator, the electrical signal delivered to each electrode, the electrical signal delivered to pairs of electrodes, the power delivered to an electrode, the power delivered to a group of electrodes, the electrical potential of an electrode relative to other electrodes, the electrical current flowing through an electrode, the power delivered to an electrode, the Temperature of an electrode, the Impedance measured between two electrodes, impedance, resistance, voltage, current, power, time, signals from sensors 603 integrated into each electrode 600, signals from sensors 603 physically separate from the electrodes, signals from sensors 603 that are remote of the active zone of any electrode, elapsed time, the time elapsed since an electrode was first energized, other signals, quantities that are derived from other measured quantities, the number of pulses of electrical output delivered to an electrode or a group of electrodes, the time-integral of measured or derived quantities, and the time-average of measured or derived quantities; a control system 615 which can use the quantities produced by the measurement system 620 to adjust the electrical power supplies 610 and switching system 605 in order to control measured or derived quantities for each of the three or more treatment electrodes 600 at the same time, or in order to control measured or derived quantities whose number is at least the number of said three or more electrodes 600. The control system 615 can determine an output level for an upcoming time period configured to achieve an electrode-specific control objective for each of the three or more treatment electrodes 600; determine a sequence of three or more grouping of said electrodes 600; determine electrical potentials to deliver to each electrode in each grouping in the sequence; determine a duration within the said upcoming time period for which each group is connected to electrical signal output in the sequence; configure said determinations of groupings, potentials, and timings such that the aggregate output level for each electrode during the said upcoming time period is equivalent to that determined for control of that electrode's electrode-specific control objective; cause the power supplies 610 and switches 605 to execute the timed sequence of grouping and assigned potentials; and then repeat the same operations for the next upcoming time period, unless a stopping criteria is met.

It is understood that the switching system 605 can be omitted and that each one of the electrodes 600 can be individually energized and de-energized by enabling and disabling power supplies 610. For example, an individual power supply component of 610 can be dedicated to one and only one of the electrodes 600, so that disabling the said power supply component puts its dedicated electrode into an electrically-passive state, and so that enabling the said power supply component and setting its output potential puts sets the output potential for its dedicated electrode.

Referring now to FIG. 8B and in accordance with one example of the present invention, an electrosurgical system is provided. Said electrosurgical system can consist of one or more indifferent electrodes 601 in contact with a body of tissue; two or more treatment electrodes placed in a body of tissue 602; a switching system 606 that can connect and disconnect said electrodes 601 and 602 to electrical power supplies 611; one or more electrical power supplies 611 that can generate electrical output signals such as differences in electrical potentials; sensors 604 configured to monitoring the electrodes 602 or the tissue in which the electrodes 602 are in contact; a measurement system 621 which can monitor the electrical output of the generator, the electrical signal delivered to each electrode, the electrical signal delivered to pairs of electrodes, the power delivered to an electrode, the power delivered to a group of electrodes, the electrical potential of an electrode relative to other electrodes, the electrical current flowing through an electrode, the power delivered to an electrode, the temperature of an electrode, the Impedance measured between two electrodes, impedance, resistance, voltage, current, power, time, signals from sensors 604 integrated into each electrode 602, signals from sensors 604 that are physically separate from the electrodes 602, signals from sensors that are remote of the active zone of any electrode 604, elapsed time, the time elapsed since an electrode was first connected to an output signal, other signals, quantities that are derived from other measured quantities, the number of pulses of electrical output delivered to an electrode or a group of electrodes, the time-integral of measured or derived quantities, and the time-average of measured or derived quantities; a control system 616 which can use the quantities produced by the measurement system 620 to adjust the electrical power supplies 611 and switching system 606 to control measured or derived quantities for each of the two or more treatment electrodes 602 at the same time. The control system 616 can determine an output level for an upcoming time period configured to achieve an electrode-specific control objective for each of the two or more treatment electrodes 602; determine a sequence of two or more groupings of both the treatment and indifferent electrodes 602 and 601, such that at least one of the groupings includes only treatment electrodes 602; determine electrical potentials to deliver to each electrode in each grouping in the sequence; determine a duration within the said upcoming time period for which each group is connected to electrical output signals in the sequence; configure said determinations of groupings, potentials, and timings such that the aggregate output level for each treatment electrode 602 during the said upcoming time period is equivalent to that determined for control of that electrode's electrode-specific control objective; cause the power supplies 611 and switches 606 to execute the timed sequence of grouping and assigned potentials; and then repeat the same operations for the next upcoming time period, unless a stopping criteria is met.

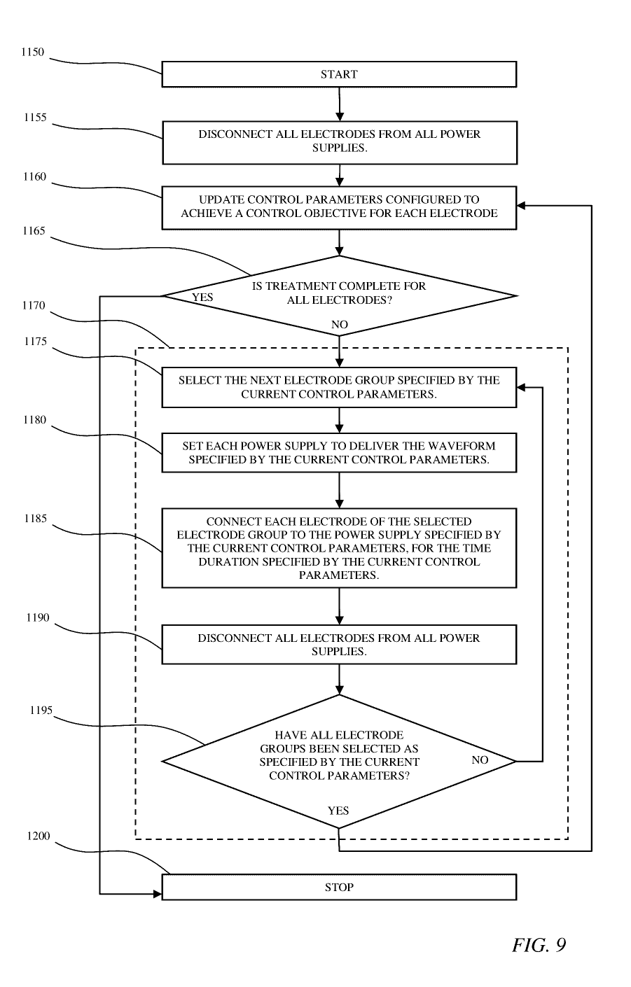

Referring now to FIG. 9 and in accordance with one example of the present invention, a control algorithm and method for control for an electrosurgical system is provided. The algorithm can be applied to a system for control of three or more electrodes placed in a living body, that is implemented without connecting additional electrodes to system power supplies. Item 1150 can represent the start of the algorithm. In step 1150, the electro surgical generator can be in an idle mode or another operating mode. In step 1150, the generator can deliver idle-type output to some or all electrodes, or the generator's switches can disconnect all electrodes from system power supplies. In step 1150, the generator can require the conditions are met before transitioning to item 1155. In step 1155, all electrodes connected to the generator can be disconnected from the electrosurgical generator's power supplies by opening the switches which connect them to those power supplies. Item 1160 can represent a control update step in which control parameters are determined, computed, changed or otherwise updated, which determine system operation in subsequent algorithm steps. Step 1160 can include a determination of an electrical output signal configured to achieve a control objective for each said three or more electrodes. Step 1160 can include a determination of an electrical output signal configured to achieve a control objective for more parameters than the number of said three or more electrodes. Said electrical output signal can contain periods in which a signal is actively connected to the electrode, and periods in which the electrode is disconnected from system power supplies by a switching system. Step 1160 can include an assignment of each electrode to one or more electrode groups, a determination of a sequence of electrode groups in a step of which only electrodes contained in the current electrode group are directly connected to a system power supply by their respective switches, and an assignment of electrical potentials, currents, waveforms, or signals to each electrode in each group for each step in the sequence in which that group is energized; said assignments and determination can be configured to produce the electrical output signal which is identical to, or configured to be equivalent to, an electrical output signal configured to achieve a control objective for each electrode. Step 1160 can use the history of measurements obtained from each electrode, for each electrode, or from sensors. For example, step 1160 can use the past Temperature value measured at each electrode to determine an output signal for each electrode configured to raise and hold its Temperature to a set value. For example, step 1160 can use past values of the Impedances measured between each electrode and other electrodes to determine an output signal for each electrode configured to prevent said Impedances from rising while energy is being delivered to said electrodes. Decision step 1165 can represent a check of criteria for discontinuing delivery of electrical output to said electrodes or for changing the type of output delivered. Step 1165 can include a check of whether the time elapsed since the start step 1150 exceeds or is equal to the value of a time setting. Step 1165 can include a check of whether a quantity which aggregates or summarizes the output delivered to one or more electrodes since the start step 1150, exceeds, is equal to, or falls below a threshold value. In step 1165, if a condition for stopping has been met, the algorithm transitions to the stop state 1200. In step 1165, if no condition for stopping has been met, the algorithm transitions to 1175. Item 1200 represents the stop state of the algorithm. In step 1200, the generator can discontinue delivering output to the electrodes. In step 1200, the generator can transition to an idle state. In step 1200, the generator can transition to another operating mode. Item 1170 collects steps 1175, 1180, 1185, 1190, 1195, which taken together constitute an algorithmic loop. Item 1170 is configured to implement duty-cycling among electrode groups. The time period over which item 1170 is executed can constitute a duty-cycle period. In step 1175, the next electrode group specified by the current control parameters, which were set in step 1160, is selected. When step 1175 follows step 1165, the first electrode group specified by the current control parameters, which were set in step 1160, is selected. Each said three or more electrodes can be assigned to one or more electrode group. In step 1180, the output signal of each of the electrosurgical system's one or more power supplies is activated as specified by the current control parameters, which were set in step 1160. In step 1180, the system power supplies can be enabled. For example, step 1180 can set the amplitude of a radiofrequency waveform produced by a system power supply. In step 1185, electrodes in the currently-selected electrode group are connected to electrical output signals for the amount of time and with the output signals specified by the current control parameters, which were set in step 1160, and all electrodes that are not in the currently-selected electrode group, are not directly connected to generator output signals. In step 1185, the switches associated with each electrode in the currently-selected electrode group can change position in order that said electrodes are connected to the power supply or power supplies specified by the current control parameters. Step 1190 is configured so that all electrodes are disconnected from electrical output signals. In step 1190, the switches that connect all electrodes to system power supplies can be opened. In step 1190, all electrodes can be disconnected from system power supplies. In step 1190, the output of all system power supplies that are connected to system power supplies can be disabled. Branch step 1195 terminates loop 1170 if it termination criteria is met, and otherwise continues to the next iteration of the loop 1170. Step 1195 can check whether the sequence of electrode groups specified by the current control parameters has been completely executed. In step 1195, if it is found that additional electrode groups have not yet been energized as specified in the sequence set by the current control parameters, the algorithm can transition to step 1175. In step 1195, if it is found that all electrode groups have been energized in sequence as specified by the current control parameters, then the algorithm can transition to step 1160.

It is understood that error-checking steps can be included in any part of the algorithm provided in FIG. 9. It is understood that detection of an error at any time in the execution of this algorithm can stop the operation of the algorithm, force transition to the stop state 1200, or force transition to another state of operation. It is understood that the order of some operations can be changed without affecting the algorithm's overall effect. For example, the order of steps 1175 and 1180 can be reversed. For example, it is understood that different specific steps can be used to implement duty-cycling step 1170 in which each electrode group is energized according to the current control parameters in a sequence such that when the electrodes of one group are energized by system power supplies, electrodes not in that group are not directly energized by system power supplies. For example, if the system power supplies can be enabled and disabled, the order of steps 1180 and 1185 can be reversed so that output levels from system power supplies are set after switches connect electrodes to the power supplies. For example, during step 1185, computation can be performed concurrently to implement subsequent control steps 1195 and 1175, and the process of de-engerizing the current electrode group and energizing the next electrode group to the specified output level, can be performed in a single step. It is understood that the flow of control structure of the algorithm can be stated differently without the affecting the algorithm's effective operation. For example, the loop 1170 can be implemented using a “for loop”. It is understood that the algorithm can be implemented in multiple computation threads either on a single processor or on multiple processors. For example, computation of control parameters which are updated in step 1155, can be performed concurrently to steps 1160, 1175, 1180, 1185, 1190, and/or 1195. It is understood that other operations, such as measurements and timing delays, can be inserted between any steps of the algorithm, or can be performed in parallel to any of the steps of the algorithm.

In accordance with one example of the present invention, a control update step 1160 is provided. Integer N can refer to a number of treatment electrodes greater than or equal to three, N≧3. Integer i can index said electrodes by taking values i=1, . . . , N. Integer M can refer to a number of electrode groups. Integer j can index said electrodes by taking values j=1, . . . , M. Each electrode group, indexed by j=1, . . . , M, can be represented by mathematical object Cⱼ, for j=1, . . . , M respectively. For a given value of j, Cⱼ is a collection of electrode indices. For example, if an electrode group with index j=3 contains electrodes indexed i=2, i=4, and i=7, this group can be denoted C₃, and its contents can be denoted (2,4,7). The contents of the same example electrode group C₃ can also be denoted (2,7,4), (4,2,7), (4,7,2), (7,2,4), or (7,4,2). An indicator variable Hᵢⱼ can be defined for all indices i=1, . . . , N and j=1, . . . , M. For a given value of i and j, indicator variable Hᵢⱼ takes value one (1) if the electrode indexed i is contained in the group indexed j, and otherwise takes value zero (0). For example, given the example electrode group C₃ containing electrodes indexed i=2, i=4, and i=7, indicator variables take the following values H₁₃=0, H₂₃=1, H₃₃=0, H₄₃=1, H₅₃=0, H₆₃=0, H₇₃=1. For each of the electrodes with respective indices i=1, . . . N, the value Uᵢ can denote an output level which is configured to achieve an control objective for electrode i by means of delivering output of level value Uᵢ to electrode i over the upcoming duty-cycle period of time duration t_{d}. Each value Uᵢ can be determined by a control algorithm. For example, each Uᵢ can be determined by an algorithm configured to control of the Temperature Tᵢ measured as electrode i, by means of adjusting the output level delivered to electrode i. In one example, an output level value Uᵢ can be a scalar variable representing an average power output; an average radiofrequency voltage amplitude; an average radiofrequency current amplitude; an average voltage; an average electrical current; an average of a function of a voltage, a current, or both; a radiofrequency voltage amplitude; a radiofrequency current amplitude; a radiofrequency power; the square of the value of a radiofrequency voltage amplitude; a voltage; a current; a power; a parameter of an electrical signal output; or a time-averaged parameter of an electrical signal output. In another example, an output level value Uᵢ can be a vector-value parameterizing a waveform or can be a representation of a waveform over the upcoming duty-cycle period t_{d}. For each electrode group, indexed j=1, . . . , M respectively, the value tⱼ can denote the amount of time that electrode group j is energized during the upcoming duty-cycle period. Each value tⱼ can have the property tⱼ≧0. The sum of the on-times t₁, . . . , t_{M} over all groups can be restricted to be less than or equal to the duty-cycle period t_{d}. An output level variable Vᵢⱼ can be defined for all indices i=1, . . . , N and j=1, . . . , M. For a given value of i and j, the variable Vᵢⱼ can denote an output level which is delivered to electrode i when electrode group j is energized during the upcoming duty-cycle period. Each variable Vᵢⱼ can be scalar-valued or vector-valued, can represent the same types of output levels and waveforms that each Uᵢ can represent. In one example, each Vᵢⱼ and Uᵢ represent the same physical quantity, such as power, for all indices i=1, . . . , N and j=1, . . . , M.

Variables Vᵢⱼ, Hᵢⱼ and tⱼ for all i=1, . . . , N and j=1, . . . , M can be determined by solving a system of equations configured to describe the equivalence of electrode-independent output waveforms parameterized by U₁, . . . U_{N} and group-duty-cycled output waveforms parameterized by Vᵢ₁, . . . , V_{iM}, t₁, . . . t_{M}, Hᵢ₁, . . . , H_{iM} over the upcoming time period t_{d}, for the purpose of achieving a control objective for each electrode i=1, . . . , N. For example, if a control algorithm employs the time-averaged output level applied to each electrode over the upcoming duty-cycle period for the purpose of achieving an control objective for each electrode, then said equations can equate the time-averaged output level due to the output waveforms parameterized by U₁, . . . , U_{N} over the upcoming time period t_{d}, to the time-averaged output level due to the output waveforms parameterized by Vᵢ₁, . . . , V_{iM}, t₁, . . . t_{M}, Hᵢ₁, . . . , H_{iM} over the upcoming time period t_{d}. The said system of equations can also include equations that describe any mutual constraint on values V₁ⱼ, . . . , V_{Nj} for a each electrode group j, given the assignment of electrodes to groups as indicated by H₁ⱼ, . . . , H_{Nj}. Said mutual constraint can be determined by the electrodes' interaction through the tissue and by the design of the electrode surgical generator. An example of such a mutual constraint include the law of electrical current conservation. Said mutual constraint can be determined by physical relations among the electrodes and the electrosurgical generator applicable when an electrode group j is energized during the upcoming duty-cycle period. A controller can determine said system of equations either by means of known physical relations among the electrodes and the electrosurgical generator, or by means of information about the physical relations among the electrodes and the electrosurgical generator that is collected in the source of energizing said electrodes. For example, said physical relations among the electrodes and the electrosurgical generator can be determined or estimated by means of measurements collected from said electrodes and system identification methods.

Said system of equations can be solved by algebraic or numerical methods. Said system of equations can be solved exactly or approximately. When said system of equations has no solution Vᵢ₁, . . . , V_{iM}, t₁, . . . t_{M}, Hᵢ₁, . . . , H_{iM} the controller can go into an error mode; or, the controller can select values for variables Vᵢ₁, . . . , V_{iM}, t₁, . . . t_{M}, Hᵢ₁, . . . , H_{iM} that produce suboptimal control results that are do not exceed other bounds. For example, the said system of equations can have no solution for which tⱼ≧0 for all j=1, . . . , M. For example, if the control objective for each electrode is to raise its temperature to a set value, Vᵢ₁, . . . , V_{iM}, t₁, . . . t_{M}, Hᵢ₁, . . . , H_{iM} can be selected such that the temperature of some or all electrodes are raised, but not above their set temperature values.

The values determined by the said system of equations Vᵢ₁, . . . , V_{iM}, t₁, . . . t_{M}, Hᵢ₁, . . . , H_{iM}, or other equation in the case where said system has no solution, can be used to determine behavior of the electrosurgical generator in the upcoming duty-cycle loop 1170. The values determined by the above system of equations Vᵢ₁, . . . , V_{iM}, t₁, . . . t_{M}, Hᵢ₁, . . . , H_{iM} can determine which electrodes are assigned to which electrode groups, how long each electrode group is energized, and the output applied to each electrode during the time when each electrode group is energized during the upcoming duty-cycle period.

In one example, the said system of equations can be written as shown below, using functions F₁, . . . , F_{N}, f₁, . . . , f_{N}, G₁, . . . , G_{M}, each of which can be scalar-valued or vector-valued.

Fᵢ(Vᵢ₁, . . . ,V_{iM},t₁, . . . t_{M},Hᵢ₁, . . . ,H_{iM})=fᵢ(Uᵢ,t_{d}), for i=1, . . . ,N (1)

Gⱼ(V₁ⱼ, . . . ,V_{Nj},H₁ⱼ, . . . ,H_{Nj})=0, for j=1, . . . ,M (2)

The above equations (2) involving G₁, . . . , G_{M}, are one example of the form of equations can be configured to describe mutual constraints among the output waveforms applied to electrodes in each electrode group while that electrode group is energized. A controller can determine functions F₁, . . . , F_{N}, f₁, . . . , f_{N}, G₁, . . . , G_{M} either by means of known physical relations among the electrodes and the electrosurgical generator, or by means of information about the physical relations among the electrodes and the electrosurgical generator that is collected in the source of energizing said electrodes. Given values for F₁, . . . , F_{N}, f₁, . . . , f_{N}, G₁, . . . , G_{M}, U₁, . . . , U_{N}, and t_{d}, above example of a system of equations can be solved for Vᵢ₁, . . . , V_{iM}, t₁, . . . t_{M}, Hᵢ₁, . . . , H_{iM}.

In one example of an electrosurgical system in accordance with the present invention, all electrode groups restricted to contains two and only two electrodes. A control update step 1160 can operate in accordance with bipolar restriction. Such an electrode group can be referred to as an “electrode pair group”, a “pair group”, an “electrode pair”, or a “pair”. One advantage of the said restriction is that two electrodes are energized in a bipolar manner, with all other electrodes floating. Another advantage of the said restriction is that when an electrode pair group is energized, each electrode can serve as the path for return currents for the other electrode. Another advantage of the said restriction is that when an electrode pair group is energized, each electrode can serve as reference electrode for the other electrode. Another advantage the said restriction is that the same relative electrical voltage can be applied to both electrodes of a group when that group is energized. Another advantage of the said restriction is that the same electrical current flows through both electrodes of a group when that group is energized. Another advantage of the said restriction is that the same electrical power loss can be ascribed to both electrodes of a group when that group is energized. For example, with the said bipolar restriction, if the total number of electrodes is N=3, there are three possible electrode groups (1,2), (2,3), and (3,1). For example, with the said bipolar restriction, if the total number of electrodes is N=4, there are six possible electrode groups (1,2), (2,3), (3,4), (4,1), (1,3), (2,4). For example, with the said bipolar restriction, if the total number of electrodes is an integer greater than or equal to three N≧3, the number of possible electrode groups is (N*(N−1)/2). With the said bipolar restriction, a control update step 1160 can use the same variable Vⱼ can parameterize the output for both electrodes in the electrode group with index j. With the said bipolar restriction, if Hᵢⱼ=1, then the restriction Vᵢⱼ=Vⱼ can be applied; this can be considered a specialization of equation (2). For example, a possibly time-varying electrical potential difference between the two electrodes in each electrode group j=1, . . . , M can be regulated to establish the output level Vⱼ.

In a more specific example of the said bipolar restriction, the controller can be configured to determine a time-average output level Uᵢ to achieve a control-objective for each electrode i. In this more specific example of the bipolar restriction with a time-average controller output, the output level for each electrode pair group j=1, . . . , M can be determined by a parameter Vⱼ for the time-average output level when that group j is energized, with all non-group electrodes floating. In this more specific example, for the upcoming duty-cycle period, the controller can determine electrode pair assignments Hᵢⱼ for i=1, . . . , N and j=1, . . . , M, electrode pair output parameters Vⱼ for j=1, . . . , M, and electrode pair on-times tⱼ by equating, for each electrode i, the time-average output level Uᵢ over duty-cycle period t_{d} is equated to the time-average output level delivered to electrode i by duty-cycling using the parameters Hᵢ₁, . . . , H_{iM}, V₁, . . . , V_{M}, t₁, . . . , t_{M}. The said equating can be performed by the following equations (3).

V₁×t₁×Hᵢ₁+ . . . +V_{M}×t_{M}×H_{iM}=Uᵢ×t_{d}, for i=1, . . . ,N (3)

In another example of an electrosurgical system in accordance with the present invention, a control update step 1160 can use a single variable V to parameterize the output assigned to each electrode group. With this single-output-level restriction, if Hᵢⱼ=1, then the restriction Vᵢⱼ=V can be applied for i=1, . . . , N and j=1, . . . , M. An advantage of the single-output-level restriction is that the electrosurgical system can contain only one power supply.

In another example of an electrosurgical system in accordance with the present invention, the said bipolar restriction, the said time-average controller output, and the said single-output-level restriction can all be applied the same system at the same time. A control update step 1160 can operate in accordance with all these specializations. An advantage of a system with all these specializations is that the system can have some or all the advantages of each of these specializations. In a system with all of the said specializations, for the upcoming duty-cycle period, the controller can determine electrode pair assignments Hᵢⱼ for i=1, . . . , N and j=1, . . . , M, the output parameter V, and electrode pair on-times tⱼ by equating, for each electrode i, the time-average output level Uᵢ over duty-cycle period t_{d} is equated to the time-average output level delivered to electrode i by duty-cycling using the parameters Hᵢ₁, . . . , H_{iM}, V, t₁, . . . , t_{M}. The said equating can be performed by the following equations (4).

V×(t₁×Hᵢ₁+ . . . +t_{M}×H_{iM})=Uᵢ×t_{d}, for i=1, . . . ,N (4)

An advantage of the N equations (4) is that, given an output level V and group assignments Hᵢ₁ for i=1, . . . , N and j=1, . . . , M, the equations are linear in the group on-times t₁, . . . , t_{M}. An advantage of equations (4) is that if the number of distinct groups M is greater than or equal to the number of electrodes N, the equations (4) can have a valid solution. An output level V and group assignments Hᵢⱼ for i=1, . . . , N and j=1, . . . , M can be selected by a control algorithm on the basis that equations (4) have a valid solution for the on-times t₁, . . . , t_{M} such that t₁≧0, . . . , and t_{M}≧0.

In a more specific example of equations (4), the number of electrodes is N=3 and equations (4) can be written as equations (5). For this example, group 1 contains electrodes 1 and 3; group 2 contains electrodes 1 and 2; and group 3 contains electrodes 2 and 3; however, it is understood that the ordering of the electrode and group indices can change without affecting the essential form of the control update step.

V×(t₁+t₂)=U₁×t_{d}

V×(t₂+t₃)=U₂×t_{d}

V×(t₃+t₁)=U₃×t_{d} (5)

In a more specific example of equations (4), the number of electrodes is N=4 and equations (4) can be written as equations (6). For this example, without loss of generality, group 1 contains electrodes 1 and 2; group 2 contains electrodes 2 and 3; group 3 contains electrodes 3 and 4; group 4 contains electrodes 4 and 1; group 5 contains electrodes 2 and 4; and group 6 contains electrodes 3 and 1; however, it is understood that the ordering of the electrode and group indices can change without affecting the essential form of the control update step. Equation (6) can have more than one solution for a given value of V.

V×(t₁+t₄+t₆)=U₁×t_{d}

V×(t₁+t₂+t₅)=U₂×t_{d}

V×(t₃+t₂+t₆)=U₃×t_{d}

V×(t₃+t₄+t₅)=U₄×t_{d} (6)

In another example of an electrosurgical system in accordance with the present invention, each electrode group is restricted such all electrodes in the group but one are set to a reference potential, and an output waveform is delivered to the said remaining one electrode in the group. A control update step 1160 can operate in accordance with this single-non-reference-electrode-per-group restriction. For example, each electrode group can contain all electrodes of which exactly one electrode is at connected to a non-reference potential while that group is energized, and all other electrodes are connected to the same reference potential while that group is energized. For example, the reference potential can either be a constant voltage or a time-varying voltage. For example, the non-reference potential applied to only one electrode in each group can either be a constant voltage or a time-varying voltage. The number of electrode groups can be equal to the total number of electrodes. Each electrode can assigned as the non-reference electrode in at least one electrode group. A controller can be configured to omit an electrode group from a duty-cycle period for the purpose of achieving an electrode-specific control objective on the non-reference electrode in that group. For an electrode group with a given non-reference electrode, a controller can include other electrode in that group so that the amount of electrical current flowing through each non-reference electrode in that group is at a level low enough that it does not substantially affect the parameter which is the subject of the electrode-specific control objective of each non-reference electrode. An advantage of the said single-non-reference-electrode-per-group restriction is that while a group is energized, the return current from the one non-reference electrode is divided among all other electrodes in that group. An advantage of the said single-non-reference-electrode-per-group restriction is that while an electrode group is energized, it can be that only the non-reference electrode carries enough electrical current to substantially affect the electrode-specific parameter which is the subject of its control objective.

In a system with the said single-non-reference-electrode-per-group restriction, a control update step 1160 can have a single variable Vⱼ which parameterizes the output level of the one non-reference electrode in each group j=1, . . . , M, and indicator variables Aᵢⱼ can be assigned value one (1) when electrode i is the one non-reference electrode in group j, and Aᵢⱼ can be assigned value zero (0) otherwise. The the upcoming duty-cycle the controller can determine electrode group assignments Hᵢⱼ for i=1, . . . , N and j=1, . . . , M, the output parameters Vⱼ, and electrode pair on-times tⱼ by equating, for each electrode i, the time-average output level Uᵢ over duty-cycle period t_{d} is equated to the time-average output level delivered to electrode i by duty-cycling using the parameters Hᵢ₁, . . . , H_{iM}, Aᵢ₁, . . . , A_{M}, V₁, . . . , V_{M}, t₁, . . . , t_{M}. The said equating can be performed by the following equations (7).

V₁×t₁×Aᵢ₁+ . . . +V_{M}×t_{M}×A_{iM}=Uᵢ×t_{d}, for i=1, . . . ,N (7)

In one example of a control update step 1160 with the said single-non-reference-electrode-per-group restriction, each electrode is assigned to exactly one group, so that the number of electrodes N is equal to the number of groups M. In this case, equations (7) can be written as equations (8).

Vᵢ×tᵢ=Uᵢ×t_{d}, for i=1, . . . ,N (8)

In another example of a control update step 1160 with the said single-non-reference-electrode-per-group restriction, each electrode is assigned to exactly one group, so that the number of electrodes N is equal to the number of groups M, and a single variable V parameterizes the output level of the one non-reference electrode for all electrode groups; that is Vⱼ=V for all j=1, . . . , M. In this example, equations (7) can be written as equations (9):

V×tᵢ=Uᵢ×t_{d}, for i=1, . . . ,N (9)

In another example of an electrosurgical system in accordance with the present invention, zero or more electrode groups can be configured in accordance with the said bipolar restriction, zero or more other electrode groups can be configured with the said unique-electrode restriction, and zero or more other electrode groups can be unrestricted. Such an example system can also be configured in accordance with the said single-output-level restriction. Combining some or all of said restrictions into a single controller can have the advantage of expanding the conditions under which the controller can achieve electrode-specific control objectives on all electrodes.

Referring now to FIG. 9 and in accordance with one example of the present invention, the algorithm provided can also be applied system for control of two or more treatment electrodes placed in a living body, with the addition of one or more indifferent electrodes placed in the same living body and with the modification that step 1600 does not specify a control objective for any indifferent electrode that is targeted by the duty-cycling implemented in step 1700. In one more specific example of the present invention, equations (1), (2), (3), (4), (5), (6), (7), (8), and (9) can be augmented to allow one or more indifferent electrodes to be included in some, but not all, of the electrode groups. In one example, the augmentation of the equations does not further restrict the overall solution by enforcing fulfillment of a control objective on the said indifferent electrodes; one advantage of such a set of augmented equations is that the indifferent can provide an additional degrees of freedom for overall solution of the equations.

In another example, the number of control objectives P can be greater than the number of electrodes N. In this example, integer k can index said control objective parameters Uₖ and take values k=1, . . . , P. The control objectives are parameterized by U₁, . . . , U_{P}. In this example, Equations (10) and (11) are analogous to equations (1) and (2). In equations (10), the functions Fₖ for k=1, . . . , P can model a physical relationship between each of the said N electrodes, indexed and the control objectives Uₖ for k=1, . . . , P. Said functions Fₖ for k=1, . . . , P can be determined by system identification methods. In equations (10), V denotes the combination of all values Vᵢⱼ for i=1, . . . , N and j=1, . . . , M, and H denotes the combination of all values Hᵢⱼ for i=1, . . . , N and j=1, . . . , M. In one example, equations (10) and (11) can have a solution if P≧M.

Fₖ(V,t₁, . . . t_{M},H)=fₖ(Uₖ,t_{d}), for k=1, . . . ,P (10)

Gⱼ(V₁ⱼ, . . . ,V_{Nj},H₁ⱼ, . . . ,H_{Nj})=0, for j=1, . . . ,M (11)

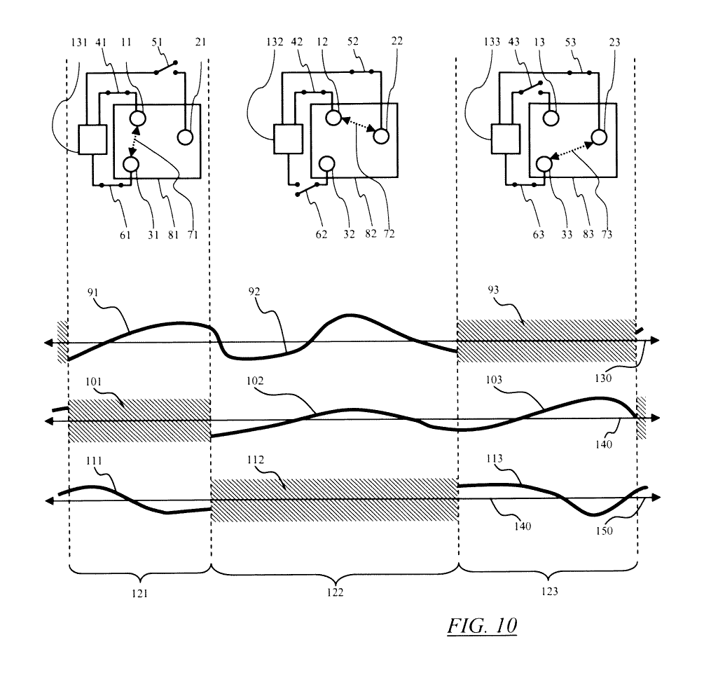

Referring now to FIG. 10 and in accordance with one example of the present invention, an example of the time-progression of electrode connections and the corresponding electrode waveforms during one duty-cycle period is presented. For instance, the depicted waveforms can be produced by the algorithmic steps contained in 1170. In this example, a three-electrode system is shown in subsequent time periods 121, 122, 123 of a duty-cycle period. One electrode is depicted as items 11, 12, 13 in time periods 121, 122, 123, respectively. A second electrode is depicted as items 21, 22, 23 in time periods 121, 122, 123, respectively. A third electrode is depicted as items 31, 32, 33 in time periods 121, 122, 123, respectively. The system power supply 3 is depicted as item 131, 132, 133 in time periods 121, 122, 123, respectively. In time period 121, the first group is active which contains electrodes 11 and 31. Electrodes 11 and 31 are connected to the system power supply 132 via switches 41 and 61, respectively. Output current 71 flows between electrodes 11 and 31. The waveform 91 on electrode 11 and the waveform 111 on electrode 31 are driven by the system power supply 132. Electrode 21 is substantially disconnected from the system power supply 132 by open switch 51. The electrical state 101 of electrode 21 is substantially determined by its surrounding in the common body in which all electrodes are placed. In time period 122, the second group is active which contains electrodes 12 and 22, which are solely energized in a bipolar manner, with electrode 32 is a passive electrical state. In time period 123, the third group is active which contains electrodes 23 and 33, which are solely energized in a bipolar manner, with electrode 13 floating. For example, a control update step 1160 using equations (4) or equations (5) can be used to determine the time-progression, switch states, and output waveforms depicted in this example. In one example, all electrodes can be treatment electrodes each associated with its own control objective, such as bringing that electrode's temperature near a set value. In another example, two of the electrodes can be treatment electrodes each associated with its own control objective, and the remaining electrode can be an indifferent electrode.

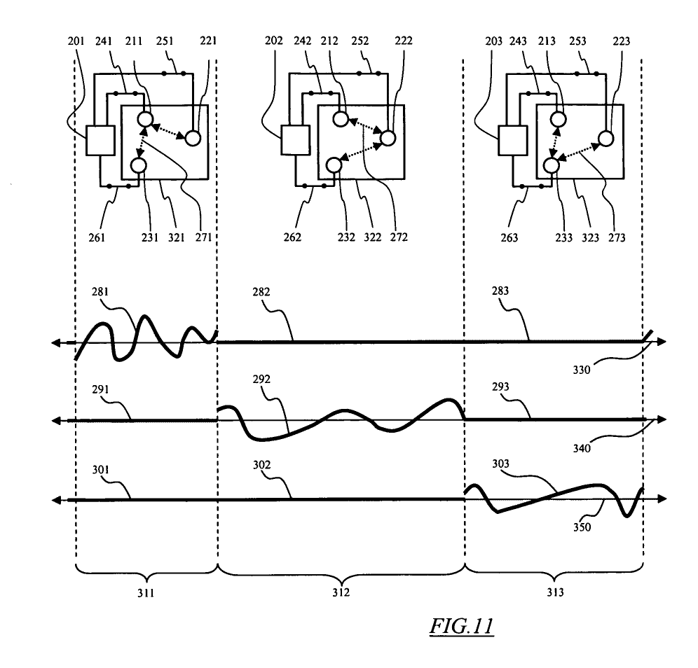

Referring now to FIG. 11 and in accordance with one example of the present invention, an example of the time-progression of electrode connections and the corresponding electrode waveforms during one duty-cycle period is presented. In this example, a three-electrode system is shown in subsequent time periods of a duty-cycle period, in a manner analogous to FIG. 10 For example, a system employing the said single-non-reference-electrode-per-group restriction can produce a duty-cycle period as shown in FIG. 11 For example, a control update step 1160 using equations (7), equations (8), or equations (9) can be used to determine the time-progression, switch states, and output waveforms depicted in this example. In one example, all electrodes can be treatment electrodes each associated with its own control objective. In another example, two of the electrodes can be treatment electrodes each associated with its own control objective, and the remaining electrode can be an indifferent electrode.

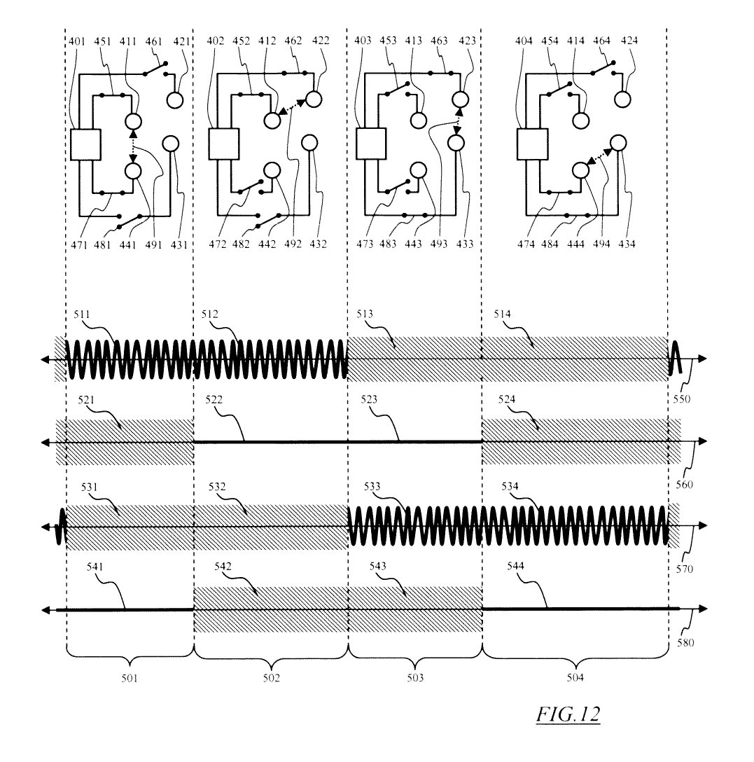

Referring now to FIG. 12 and in accordance with one example of the present invention, an example of the time-progression of electrode connections and the corresponding electrode waveforms during one duty-cycle period is presented. In this example, a four-electrode system is shown in subsequent time periods of a duty-cycle period, in a manner analogous to FIG. 10 For example, a control update step 1160 using equations (5) or equations (6) can be used to determine the time-progression, switch states, and output waveforms depicted in this example. In one example, all electrodes can be treatment electrodes each associated with its own control objective. In another example, three of the electrodes can be treatment electrodes each associated with its own control objective, and the remaining electrode can be an indifferent electrode.

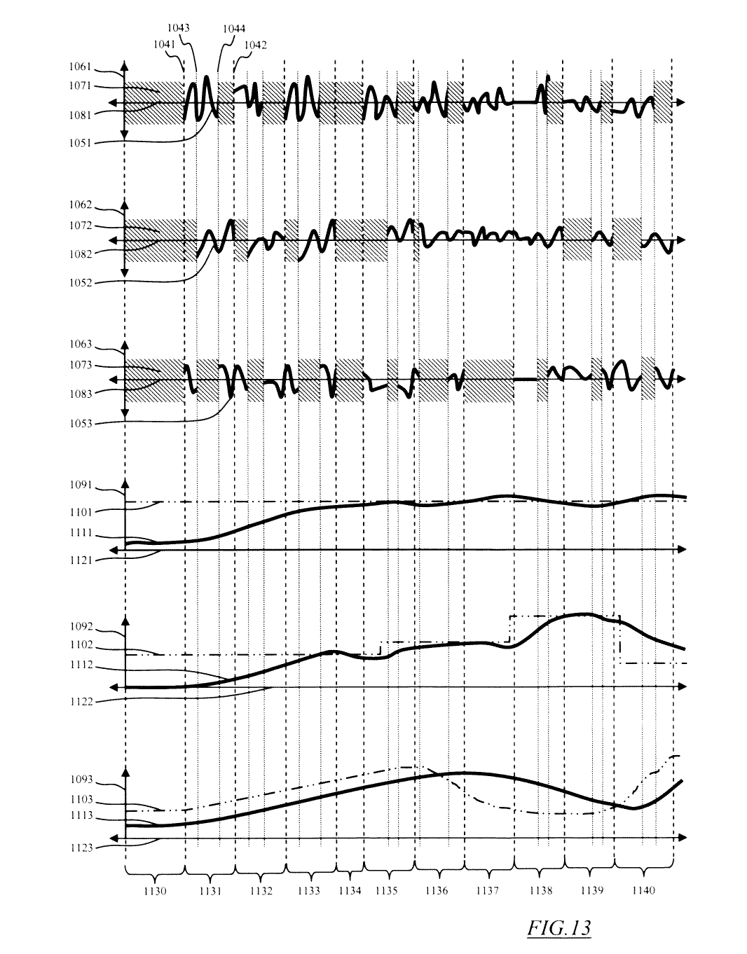

Referring now to FIG. 13 and in accordance with one example of the present invention, an example of the time-progression of electrode waveforms and controlled electrode-specific parameters during multiple subsequent duty-cycle periods 1130-1140 are presented. In this example, a three-electrode system is presented. Axis 1061 plots the output waveform, such as an electrical potential for the first electrode over time axis 1081. On these axes 1061, 1081, hatched regions, such as 1071, indicate a floating state for the first electrode, and lines such as 1051 indicate the output waveform for the first electrode. Analogous designations are used for the second electrode on axis 1072, 1082. Analogous designations are used for the third electrode on axis 1073, 1083. Axis 1091 plots the electrode-specific parameter subject to the control objective for the first electrode. For example, axis 1091 can measure the temperature of the first electrode. Axis 1121 measures time. Axes 1081, 1082, 1083, 1121, 1122, 1123 are aligned such that points which align vertical indicate the same point in time. Dash-double-dotted line 1101 plotted on axis 1091, 1121 shows the control objective for the first electrode's parameter, such as its temperature. Lines 1102 and 1103 show the control objective for the second and third electrodes' parameters, respectively. Solid line 1111 plots the measured parameter that is the subject of the first electrode's control objective; for example line 1111 can plot the temperature measured at the first electrode. Solid lines 1112 and 1113 plot the measured parameters that are the subject of the second and third electrodes' control objectives, respectively; for example, line 1112 can plot the temperature measured at the second electrode, and line 1113 can plot the temperature measured at the second electrode. Over multiple subsequent duty-cycle periods 1130-1140, the system can adjust the electrical output delivered to each electrode so that each measured parameter 1111, 1112, 1113 track with their corresponding control objective 1101, 1102, 1103, respectively.

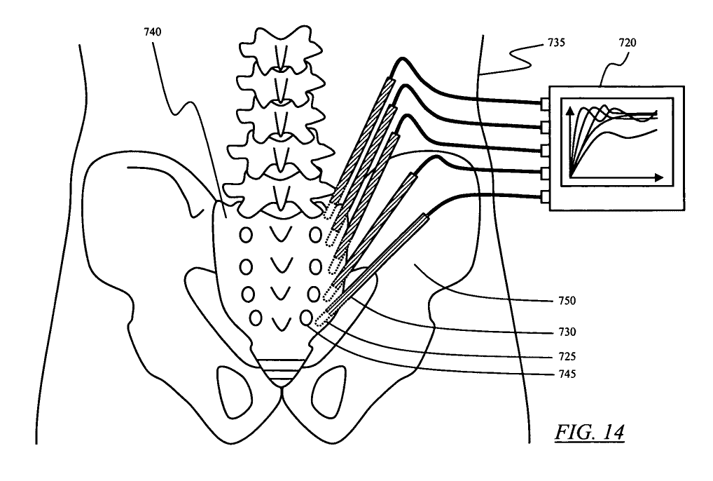

Referring now to FIG. 14 and in accordance with one example of the present invention, an example the five electrodes placed in the sacroiliac region of the human body is presented.

Referring now to FIG. 15 and in accordance with one example of the present invention, an example of four treatment electrodes placed in the spinal region of the human body is presented. For example, some or all of the electrodes can be placed near medial branch nerves.

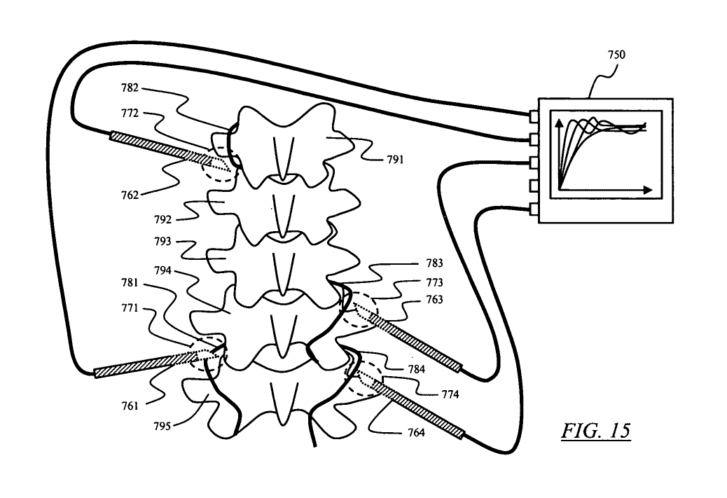

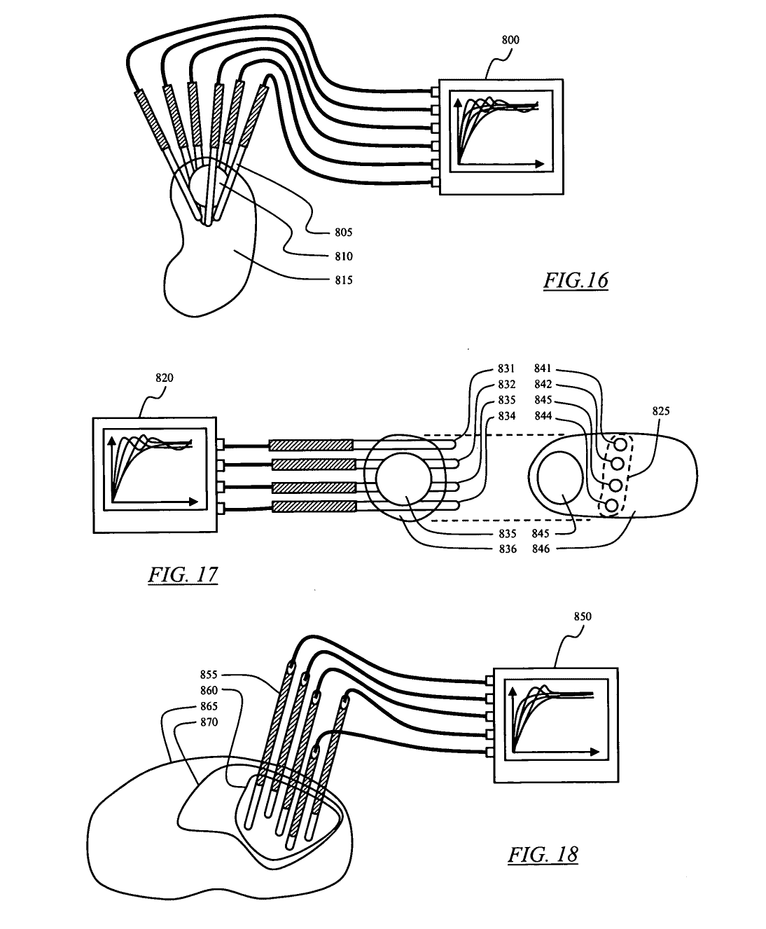

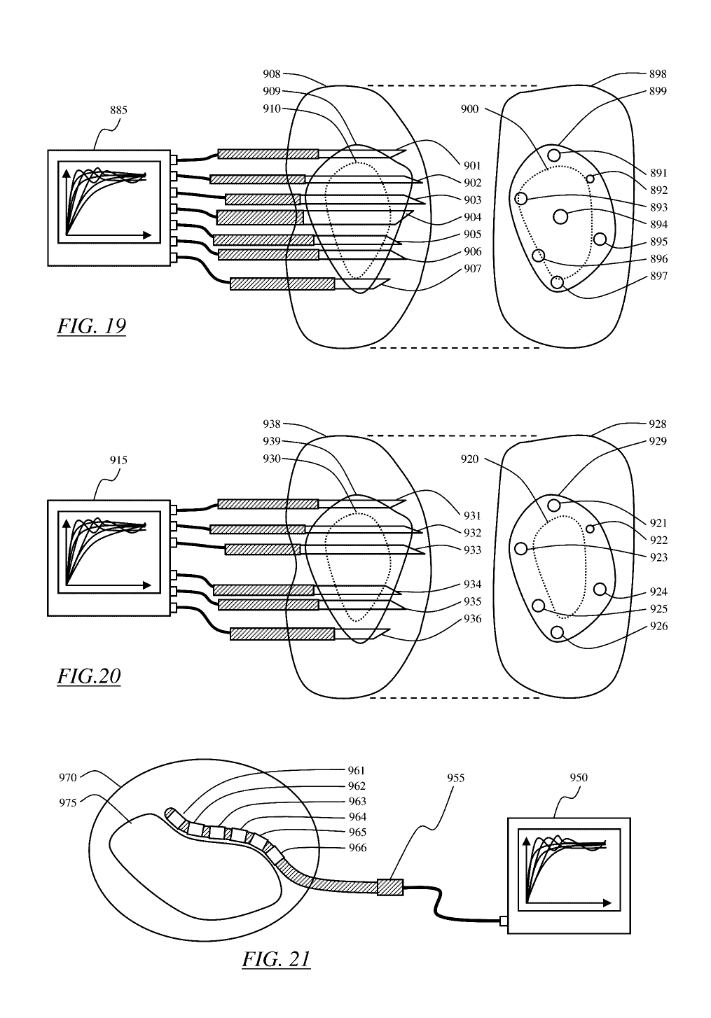

Referring now to FIGS. 16, 17, 18, 19, and FIG. 20 and in accordance with one example of the present invention, examples are presented of four, five, six, and seven electrodes placed in organs of the human body. For example, electrodes can be placed in the kidney or liver. For example, electrodes can be placed percutaneously, laproscopically, or in an open surgical procedure. For example, electrode placement can be configured to surround an tumor. For example, electrode placement can be configured to reduce blood flow in a region, such as that containing a tumor, to facilitate surgical resection of that region. For example, electrodes can be placed around a substructure of an organ, such as a tumor. For example, electrodes can be placed within a substructure of an organ, such as a tumor. It is understood that more than seven electrodes can also be used in manner shown in FIGS. 16, 17, 18, 19, and 20.

Referring now to FIG. 21 and in accordance with one example of the present invention, an example of electrodes that are integrated into a common structure is presented. For example, said structure can be a probe, a needle, a tissue-piecing elongated shaft, catheter, steerable catheter, or a laparoscopic instrument. For example, said structure can be conformed or conformable to a target structure or structures. For example, said structure can be placed in the sacroiliac region of the spine.

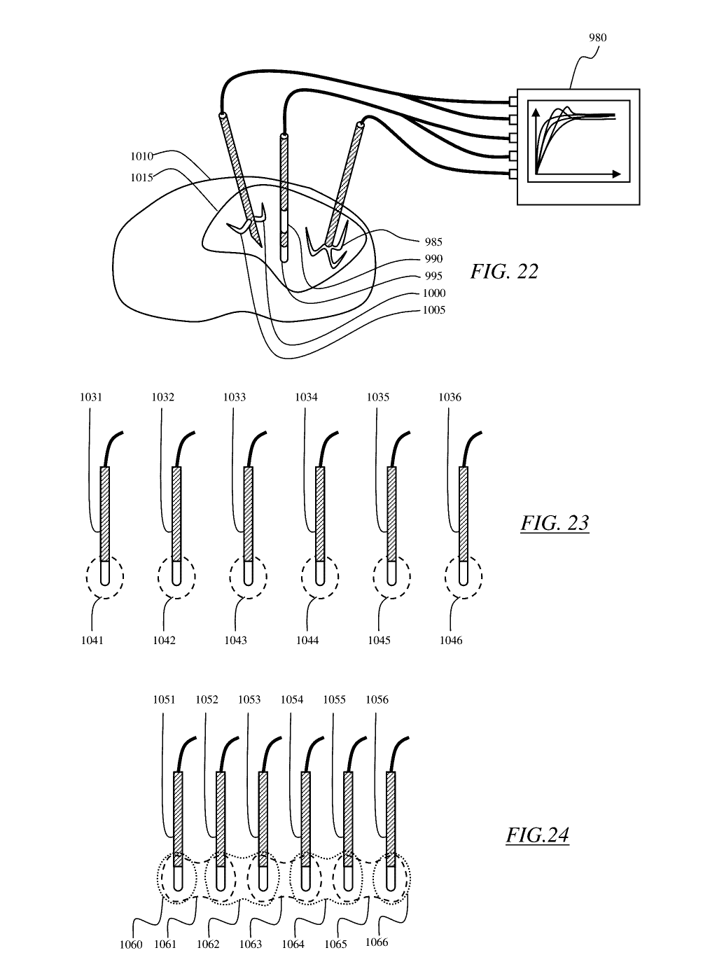

Referring now to FIG. 22 and in accordance with one example of the present invention, an example of a system that incorporates electrodes of different shapes, such as hook-shaped or umbrella-shaped, is presented.

Referring now to FIG. 23 and in accordance with one example of the present invention, an example of a system is presented in which electrodes are spaced sufficiently far apart that their respective spheres of influence do not overlap. For example, electrodes can be placed such that the electrical current and/or heating pattern is not substantially focused between the uninsulated tips of any electrodes.

Referring now to FIG. 24 and in accordance with one example of the present invention, an example of a system is presented in which electrodes are spaced sufficiently close together that their respective spheres of influence do overlap. For example, electrodes can be placed such that the electric current and/or heating pattern is substantially focused between some or all of the electrodes. It is understood that a system can incorporate electrode placements configured such that some electrodes' regions of influence overlap, and some electrode's regions of influence do not overlap.

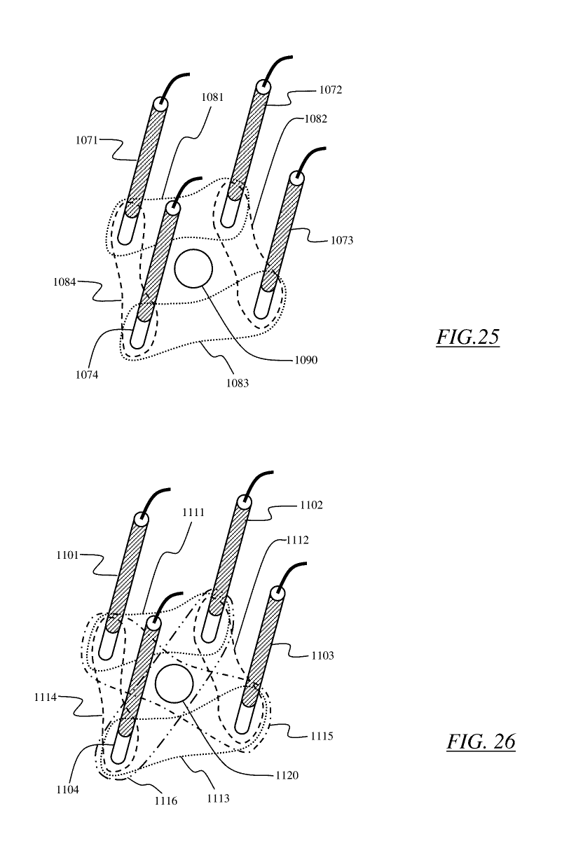

Referring now to FIG. 25 and in accordance with one example of the present invention, an example of a system is presented in which electrodes are placed to surround a structure and the electrode groups are restricted to influence tissue surrounding that structure, but not within that structure.

Referring now to FIG. 26 and in accordance with one example of the present invention, an example of a system is presented in which electrodes are placed to surround a structure and the electrode groups influence tissue both surrounding that structure and within that structure.

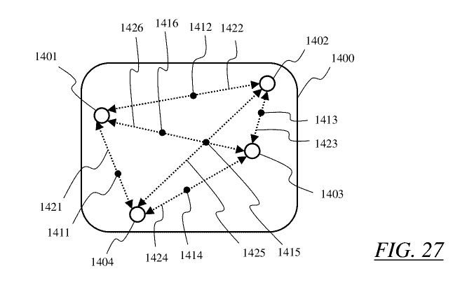

Referring now to FIG. 27 and in accordance with one example of the present invention, an example of an electrosurgical system is presented in schematic form in which the number of parameters controlled is greater than the number of electrodes 1401, 1402, 1403, 1404. In the presented example, four electrodes 1401, 1402, 1403, 1404 are placed in tissue 1400, and six sensors 1411, 1412, 1413, 1414, 1415, 1416 are placed in the said tissue 1400. The electrodes 1401, 1402, 1403, 1404 and probes 1411, 1412, 1413, 1414, 1415, 1416 are connected to a generator, such as generator 625 of FIG. 7A, that is not depicted in FIG. 27, but should be familiar one skilled in the art. In one embodiment, the said sensors 1411, 1412, 1413, 1414, 1415, 1416 can be temperature probes. A controller can energize pairs of electrodes 1401, 1402, 1403, 1404 in sequence, where each step of the sequence can be assigned to one of six possible electrode pairs. In one example, the arrow-headed lines 1421, 1422, 1423, 1424, 1425, 1426 can each represent the path that electrical current flows between a pair of electrode when that pair is energized in the said sequence. In another example, the arrow-headed lines 1421, 1422, 1423, 1424, 1425, 1426 can each represent schematically a region in which thermal energy is deposited by a pair of electrodes when that pair is energized in the said bipolar sequence. For instance, line 1421 is associated with the pairing of electrodes 1401 and 1404. Electrode pairs include the pair of electrode 1401 and electrode 1402, the pair of electrode 1401 and electrode 1403, the pair of electrode 1401 and electrode 1404, the pair of electrode 1402 and electrode 1403, the pair of electrode 1402 and electrode 1404, and the pair of electrode 1403 and electrode 1404. In one example, a controller can configure the sequence of pairs of electrodes 1401, 1402, 1403, 1404, the timing of each step of that sequence, and/or the output signal applied to the pair selected during each step of that sequence to control the parameters measured at all probes 1411, 1412, 1413, 1414, 1415, 1416 at the same time. In one example, the controller can make said configuration by solving equations (10) and (11). For example, a controller can control a distribution of temperatures measured by probes 1411, 1412, 1413, 1414, 1415, 1416. In one example, a controller can configure the sequence of pairs of electrodes 1401, 1402, 1403, 1404, the timing of each step of that sequence, and/or the output signal applied to the pair selected during each step of that sequence, to control the impedance measured between each pair of electrodes. In one example, a controller can configure the sequence of pairs of electrodes 1401, 1402, 1403, 1404, the timing of each step of that sequence, and/or the output signal applied to the pair selected during each step of that sequence, to control the impedance measured between more than one pair of electrodes. In one example, a controller can configure the sequence of pairs of electrodes 1401, 1402, 1403, 1404, the timing of each step of that sequence, and/or the output signal applied to the pair selected during each step of that sequence, to control the impedance measured between more than two pairs of electrodes. In one example, a controller can configure the sequence of pairs of electrodes 1401, 1402, 1403, 1404, the timing of each step of that sequence, and/or the output signal applied to the pair selected during each step of that sequence, to control the current that flows between a pair of electrodes, for some or all pairs of electrodes. In one example, a controller can configure the sequence of pairs of electrodes 1401, 1402, 1403, 1404, the timing of each step of that sequence, and/or the output signal applied to the pair selected during each step of that sequence, to control the power delivered between a pair of electrodes, for all pairs of electrodes. In one example, a controller can configure the sequence of pairs of electrodes 1401, 1402, 1403, 1404, the timing of each step of that sequence, and/or the output signal applied to the pair selected during each step of that sequence, to control the power delivered between a pair of electrodes, for a number of pairs of electrodes that is at least the number of electrodes in total activated during said sequence. It is understood that more general groupings of electrodes 1401, 1402, 1403, 1404 can allow a controller to control other parameters. One example of said more general grouping is a grouping is which one electrode is at one potential and multiple other electrodes are at another potential. In one example, parameters under control can include parameters measured in the electrodes 1401, 1402, 1403, 1404 themselves. It is understood that a greater number of parameters than the number of electrodes can be controlled for a similar configuration in which the number of electrodes is at least three. In one example, the

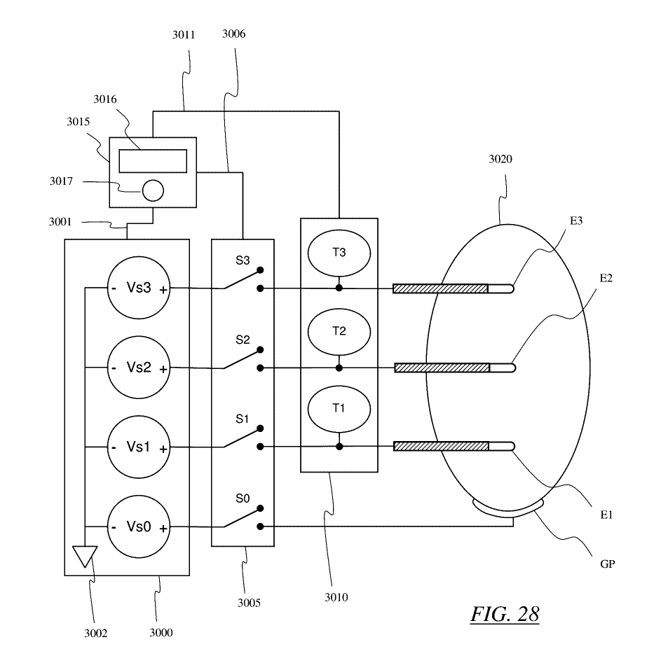

Referring now to FIG. 28 and in accordance with one example of the present invention, an example of a system configured for delivery of electrical output to a living body 3020 is presented. In this example, the power supply unit 3000 is configured to produce more than two poles of electrical output to which electrodes E1, E2, E3 and reference ground pad GP can be connected via the switching system 3005. Each electrode can be connected and disconnected from the power supply 3000 by closing and opening switches S1, S2, S3, respectively. The reference ground pad GP can be connected and disconnected from the power supply 3000 by closing and opening switch S0. The measurement system 3010 can collect a measurement T1, T2, T3 for each electrode E1, E2, E3 respectively. In one example, measurements T1, T2, T3 can include a temperature. In one example, measurements T1, T2, T3 can include a current. In one example, measurements T1, T2, T3 can include a voltage. In one example, measurements T1, T2, T3 can include a power. In one example, measurements T1, T2, T3 can include the average current over more than one step in a sequence of switch states. In one example, measurements T1, T2, T3 can include the average voltage over more than one step in a sequence of switch states. In one example, measurements T1, T2, T3 can include the average power over more than one step in a sequence of switch states. In one example, measurements T1, T2, T3 can include an impedance.

The controller 3015 is connected to the power supply 3000, switches 3005, measurement system 3010. The controller can coordinate the actions of the power supply 3000, switches 3005, measurement system 3010. For example, the controller can implement feedback control of the power supply 3000 and switches 3005 based on measurements T1, T2, T3 from the measurement system 3015.

Power supply 3000 consists of voltage supplies Vs0, Vs1, Vs2, Vs3, referenced to a common reference potential 3002. The controller 3015 can control each of the voltage supplies independently. In one example, each voltage supply can produce a different output signal. In one example, the voltage supplies Vs0, Vs1, Vs2, Vs3 can produce radiofrequency signals. In one example, a two-pole system can be produced by setting voltage supplies Vs0, Vs1, Vs2, Vs3 such that each supply produces one of two output signals. For example, in one specific example of a two-pole system, a sequence of power supply settings can include a step in which Vs0=Vs3=V+ and Vs1=Vs2=V−, and another step in which Vs1=Vs3=V+ and Vs0=Vs2=V−, such that V+ and V− can be set individually by the controller. In one example, a three-pole system can be produced by setting voltage supplies Vs0, Vs1, Vs2, Vs3 such that each supply produces one of three output signals. For example, in one specific example of a three-pole system, a sequence of power supply settings can include a step in which Vs0=V+, Vs1=V−, and Vs2=Vs3=V*; and a step in which Vs1=V+, Vs2=V−, and Vs3=Vs0=V*; where V+, V−, and V* can be set individually by the controller. In one example, a four-pole system can be produced by independently assigning output signals to each of Vs0, Vs1, Vs2, Vs3.

It is understood that, in one example, the number of electrodes can be any number N that is at least two when a reference ground pad GP is used. In this case, the electrodes E1, . . . , EN, where N≧2, can be respectively associated with measurement elements, switches and voltage supplies is a manner that is analogous to that shown from electrodes E1, E2, E3; measurement elements T1, T2, T3; switches S1, S2, S3; voltage supplies Vs1, Vs2, Vs3, as shown in FIG. 28. In this example, the system can produce from two to N+1 output poles, inclusive. It is understood that, in another example, the number of electrodes N can be any number that is at least three when the reference ground pad GP is omitted from the system. In this case, the electrodes E1, . . . , EN, where N≧3, can be respectively associated with measurement elements, switches and voltage supplies is a manner that is analogous to that shown from electrodes E1, E2, E3; measurement elements T1, T2, T3; switches S1, S2, S3; voltage supplies Vs1, Vs2, Vs3, as shown in FIG. 28. In this example, the system can produce from two to N output poles, inclusive.

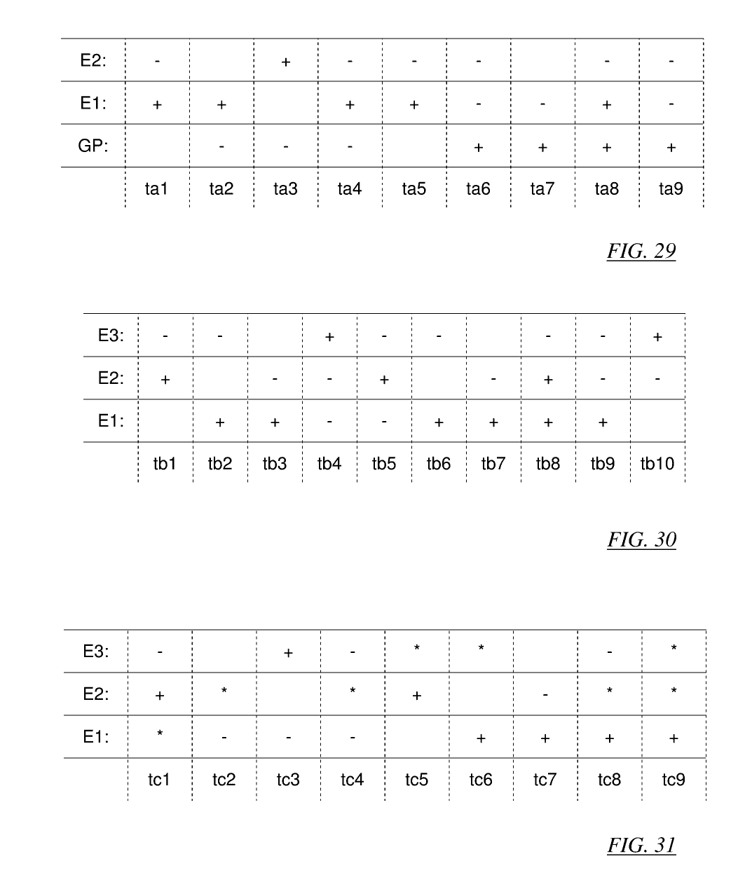

Referring now to FIG. 29, in accordance with one example of the present invention, a sequence is presented in which two electrodes E1, E2 and a ground pad GP are connected and disconnected from two electrical output poles of a system configured to deliver electrical energy to a living body. The said two electrical output poles are identified as + and −. Each step of the sequence occurs in one of the time periods ta1, ta2, ta3, ta4, ta5, ta6, ta7, ta8, and ta9. During the sequence, electrical signals are applied to each of the said electrical output poles. In one more specific example, the sequence can be produced by the system shown in FIG. 1. In another more specific example, the sequence can be produced by the system shown in FIG. 7B. In another more specific example, the sequence can be produced by the system shown in FIG. 8B. In another more specific example, the sequence can be produced by the system shown in FIG. 28.

In the first step of the sequence, during time period ta1, the ground pad GP is not connected to any output pole, electrode E1 is connected to output pole +, and electrode E2 is connected to output pole −. In the next step, during time period ta2, the ground pad GP is connected to output pole −, electrode E1 is connected to output pole +, and electrode E2 is not connected to any output pole. In the next step, during time period ta3, the ground pad GP is connected to output pole −, electrode E1 is not connected to any output pole, and electrode E2 is connected to output pole +. In the next step, during time period ta4, the ground pad GP is connected to output pole −, electrode E1 is connected to output pole +, and electrode E2 is connected to output pole −. In the next step, during time period ta5, the ground pad GP is not connected to any output pole, electrode E1 is connected to output pole +, and electrode E2 is connected to output pole −. This configuration of connections is a repetition of the configuration of the first step ta1. In the next step, during time period ta6, the ground pad GP is connected to output pole +, electrode E1 is connected to output pole −, and electrode E2 is connected to output pole −. In the next step, during time period ta7, the ground pad GP is connected to output pole +, electrode E1 is connected to output pole −, and electrode E2 is not connected to any output pole. In the next step, during time period ta8, the ground pad GP is connected to output pole +, electrode E1 is connected to output pole +, and electrode E2 is connected to output pole −. In the next step, during time period ta9, the ground pad GP is connected to output pole +, electrode E1 is connected to output pole −, and electrode E2 is connected to output pole −. This configuration of connections is a repetition of the configuration in time period ta6.

One advantage of the sequence presented in FIG. 29 is that it contains at least one step in which at least three of the following elements are connected to the system's output poles at the same time: electrode E1, electrode E2, and ground pad GP. Another advantage of the sequence presented in FIG. 29 is that contains at least one step in which the ground pad GP is not connected to an electrical output pole, and at least one other step in which the ground pad is connected to an electrical output pole. Another advantage of the sequence presented in FIG. 29 is that it contains steps in which the connections from electrodes and the ground pad to the output pole are repeated. Another advantage of the sequence presented in FIG. 29 is that it contains at least one step in which an electrode is connected to each of the electrical output poles with the ground pad not connected to any electrical output pole; it contains at least one other step in which the ground pad is connected to one of the electrode output poles and at least one electrode is connected to another output pole; and at least one of these steps is repeated in the sequence.

In one example of the system presented in FIG. 29, the output poles of the system can include a radiofrequency output pole. In one example, the output poles of the system can be connected to a radiofrequency power supply. In another example, the output poles of the system can be connected to a pulsed radiofrequency power supply. In one example, the sequence can be automated. In another example, the steps in the sequence can occur rapidly relative to the physical dynamics of parameters that are being controlled. In another example, the steps of the sequence can each last less than 100 milliseconds. In another example, the steps of the sequence can each last less than 1 second. In another example, the steps of the sequence can each last less than 3 seconds. In another example, the steps of the sequence can each less than 3 seconds. In one example, the total duration of the switching sequence can up to 10 seconds. In another example, the total duration of the switching sequence can up to 30 seconds. In another example, the total duration of the switching sequence can up to 60 seconds. In another example, the total duration of the switching sequence can up to 120 seconds. In another example, the total duration of the switching sequence can up to 150 seconds. In another example, the total duration of the switching sequence can up to 180 seconds. In another example, the total duration of the switching sequence can up to 600 seconds. In another example, the total duration of the switching sequence can up to 1800 seconds. In another example, the total duration of the switching sequence can be greater than 1800 seconds.

In another example, the sequence shown in FIG. 29 can continue beyond the steps shown and can include steps which contain additional type of connections from the output poles +, − to the electrodes E1, E2 and ground pad GP. In another example, the sequence of connection from output poles to electrodes and the ground pad can differ from the specific sequence shown in FIG. 29. For example, the number of steps in the sequence can be different that the number shown in FIG. 29. For example, the number of steps in the sequence can be configured to a control objective and/or a clinical objective. For example, the identity of the connections in each step can be different than the identities shown in FIG. 29. For example, the identity of the connections in each step can be configured to achieve a control and/or clinical objective.

The example shown in FIG. 29 can be generalized such that the number of electrodes N can be any number that is at least two N≧2. In another example, the number of steps in the sequence can be any number greater than one. In another example, a sequence can contains hundreds of steps. In another example, a sequence can contains thousands of steps. In another example, the sequence can consist of repeated configurations of connection from the output poles to the electrodes and/or ground pad. In another example, the sequence can be periodic in its steps. In another example, the number of output poles in the sequence can be any number that is at least 2. For example, three output poles can be used.

Referring now to FIG. 30, in accordance with one example of the present invention, a sequence is presented in which three electrodes E1, E2, E3 are connected and disconnected from two electrical output poles of a system configured to deliver electrical energy to a living body. The said two electrical output poles are identified as + and −. Each step of the sequence occurs in one of the time periods tb1, tb2, tb3, tb4, tb5, tb6, tb7, tb8, tb9 and tb10. During the sequence, electrical signals are applied to each of the said electrical output poles. In one more specific example, the sequence can be produced by the system shown in FIG. 1. In another more specific example, the sequence can be produced by the system shown in FIG. 7A. In another more specific example, the sequence can be produced by the system shown in FIG. 8A. In another more specific example, the sequence can be produced by the system shown in FIG. 28.

In the first step of the sequence, during time period tb1, electrode E1 is not connected to any output pole, electrode E2 is connected to output pole +, and electrode E3 is connected to output pole −. In the next step, during time period tb2, electrode E1 is connected to output pole +, electrode E2 is not connected to any output pole, and electrode E3 is connected to output pole −. In the next step, during time period tb3, electrode E1 is connected to output pole +, electrode E2 is connected to output pole −, and electrode E3 is not connected to any output pole. In the next step, during time period tb4, electrode E1 is connected to output pole −, electrode E2 is connected to output pole −, and electrode E3 is connected to output pole +. In the next step, during time period tb5, electrode E1 is connected to output pole −, electrode E2 is connected to output pole +, and electrode E3 is connected to output pole −. In the next step, during time period tb6, electrode E1 is connected to output pole +, electrode E2 is not connected to any output pole, and electrode E3 is connected to output pole −. In the next step, during time period tb7, electrode E1 is connected to output pole +, electrode E2 is connected to output pole −, and electrode E3 is not connected to any output pole. This is is the same connection configuration as that in time period tb3. In the next step, during time period tb8, electrode E1 is connected to output pole +, electrode E2 is connected to output pole +, and electrode E3 is connected to output pole −. In the next step, during time period tb9, electrode E1 is connected to output pole +, electrode E2 is connected to output pole −, and electrode E3 is connected to output pole −. In the next step, during time period tb10, electrode E1 is not connected to any output pole, electrode E2 is connected to output pole −, and electrode E3 is connected to output pole +.

One advantage of the sequence presented in FIG. 30 is that it contains at least one step in which at least three of the electrodes E1, E2, E3 are connected to the system's output poles at the same time. Another advantage of the sequence presented in FIG. 30 is that a ground pad is not used.

In one example of the system presented in FIG. 30, the output poles of the system can include a radiofrequency output pole. In one example, the output poles of the system can be connected to a radiofrequency power supply. In another example, the output poles of the system can be connected to a pulsed radiofrequency power supply. In one example, the sequence can be automated. In another example, the steps in the sequence can occur rapidly relative to the physical dynamics of parameters that are being controlled. In another example, the steps of the sequence can each last less than 100 milliseconds. In another example, the steps of the sequence can each last less than 1 second. In another example, the steps of the sequence can each last less than 3 seconds. In another example, the steps of the sequence can each less than 3 seconds. In one example, the total duration of the switching sequence can up to 10 seconds. In another example, the total duration of the switching sequence can up to 30 seconds. In another example, the total duration of the switching sequence can up to 60 seconds. In another example, the total duration of the switching sequence can up to 120 seconds. In another example, the total duration of the switching sequence can up to 150 seconds. In another example, the total duration of the switching sequence can up to 180 seconds. In another example, the total duration of the switching sequence can up to 600 seconds. In another example, the total duration of the switching sequence can up to 1800 seconds. In another example, the total duration of the switching sequence can be greater than 1800 seconds.

In another example, the sequence shown in FIG. 30 can continue beyond the steps shown and can include steps which contain additional type of connections from the output poles +, − to the electrodes E1, E2, and E3. In another example, the sequence of connection from output poles to electrodes and the ground pad can differ from the specific sequence shown in FIG. 30. For example, the number of steps in the sequence can be different that the number shown in FIG. 30. For example, the number of steps in the sequence can be configured to a control objective and/or a clinical objective. For example, the identity of the connections in each step can be different than the identities shown in FIG. 30. For example, the identity of the connections in each step can be configured to achieve a control and/or clinical objective.

The example shown in FIG. 30 can be generalized such that the number of electrodes N can be any number that is at least three N≧3. In another example, the number of steps in the sequence can be any number greater than one. In another example, a sequence can contains hundreds of steps. In another example, a sequence can contains thousands of steps. In another example, the sequence can consist of repeated configurations of connection from the output poles to the electrodes and/or ground pad. In another example, the sequence can be periodic in its steps. In another example, the number of output poles in the sequence can be any number that is at least 2. For example, three output poles can be used.

Referring now to FIG. 31, in accordance with one example of the present invention, a sequence is presented in which three electrodes E1, E2, E3 are connected and disconnected from three electrical output poles of a system configured to deliver electrical energy to a living body. The said three electrical output poles are identified as +, −, and *. Each step of the sequence occurs in one of the time periods tc1, tc2, tc3, tc4, tc5, tc6, tc7, tc8, tc9 and tc10. During the sequence, electrical signals are applied to each of the said electrical output poles. In one more specific example, the sequence can be produced by the system shown in FIG. 1. In another more specific example, the sequence can be produced by the system shown in FIG. 7A. In another more specific example, the sequence can be produced by the system shown in FIG. 8A. In another more specific example, the sequence can be produced by the system shown in FIG. 28.

In the first step of the sequence, during time period tc1, electrode E1 is connected to output pole *, electrode E2 is connected to output pole +, and electrode E3 is connected to output pole −. In the next step, during time period tc2, electrode E1 is connected to output pole −, electrode E2 is connected to output pole *, and electrode E3 is not connected to any output pole. In the next step, during time period tc3, electrode E1 is connected to output pole −, electrode E2 is not connected to any output pole, and electrode E3 is connected to output pole +. In the next step, during time period tc4, electrode E1 is connected to output pole −, electrode E2 is connected to output pole *, and electrode E3 is connected to output pole −. In the next step, during time period tc5, electrode E1 is not connected to any output pole, electrode E2 is connected to output pole +, and electrode E3 is connected to output pole *. In the next step, during time period tc6, electrode E1 is connected to output pole +, electrode E2 is not connected to any output pole, and electrode E3 is connected to output pole *. In the next step, during time period tc7, electrode E1 is connected to output pole +, electrode E2 is connected to output pole −, and electrode E3 is not connected to any output pole. In the next step, during time period tc8, electrode E1 is connected to output pole +, electrode E2 is connected to output pole *, and electrode E3 is connected to output pole −. In the next step, during time period tc9, electrode E1 is connected to output pole +, electrode E2 is connected to output pole *, and electrode E3 is connected to output pole *.

One advantage of the sequence presented in FIG. 31 is that it contains at least one step in which at least three of the electrodes E1, E2, E3 are connected to the system's output poles at the same time. Another advantage sequence presented in FIG. 31 is that a ground pad is not connected to the system output poles in any step.

In one example of the system presented in FIG. 31, the output poles of the system can include a radiofrequency output pole. In one example, the output poles of the system can be connected to a radiofrequency power supply. In another example, the output poles of the system can be connected to a pulsed radiofrequency power supply. In one example, the sequence can be automated. In another example, the steps in the sequence can occur rapidly relative to the physical dynamics of parameters that are being controlled. In another example, the steps of the sequence can each last less than 100 milliseconds. In another example, the steps of the sequence can each last less than 1 second. In another example, the steps of the sequence can each last less than 3 seconds. In another example, the steps of the sequence can each less than 3 seconds. In one example, the total duration of the switching sequence can up to 10 seconds. In another example, the total duration of the switching sequence can up to 30 seconds. In another example, the total duration of the switching sequence can up to 60 seconds. In another example, the total duration of the switching sequence can up to 120 seconds. In another example, the total duration of the switching sequence can up to 150 seconds. In another example, the total duration of the switching sequence can up to 180 seconds. In another example, the total duration of the switching sequence can up to 600 seconds. In another example, the total duration of the switching sequence can up to 1800 seconds. In another example, the total duration of the switching sequence can be greater than 1800 seconds.

In another example, the sequence shown in FIG. 31 can continue beyond the steps shown and can include steps which contain additional type of connections from the output poles +, −, * to the electrodes E1, E2, and E3. In another example, the sequence of connection from output poles to electrodes and the ground pad can differ from the specific sequence shown in FIG. 31. For example, the number of steps in the sequence can be different that the number shown in FIG. 31. For example, the number of steps in the sequence can be configured to a control objective and/or a clinical objective. For example, the identity of the connections in each step can be different than the identities shown in FIG. 31. For example, the identity of the connections in each step can be configured to achieve a control and/or clinical objective.

The example shown in FIG. 31 can be generalized such that the number of electrodes N can be any number that is at least three N≧3. In another example, the number of steps in the sequence can be any number greater than one. In another example, a sequence can contains hundreds of steps. In another example, a sequence can contains thousands of steps. In another example, the sequence can consist of repeated configurations of connection from the output poles to the electrodes and/or ground pad. In another example, the sequence can be periodic in its steps. In another example, the number of output poles in the sequence can be any number that is at least 2. For example, four output poles can be used.

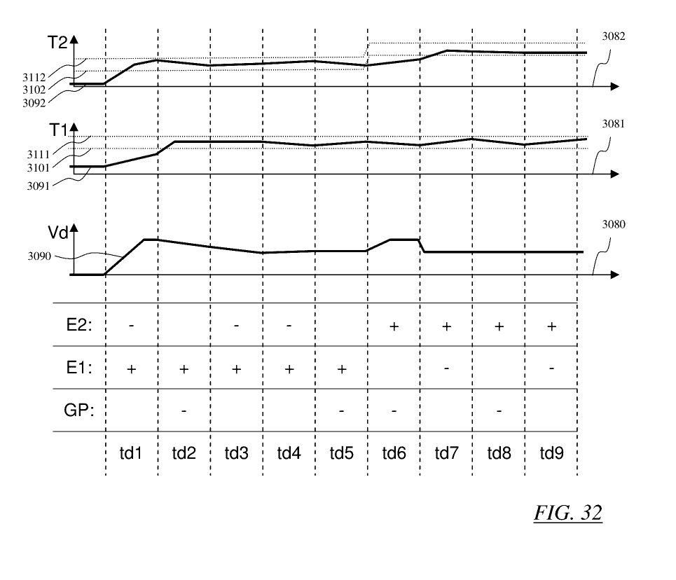

Referring now to FIG. 32, in accordance with one example of the present invention, a sequence is presented in which two electrodes E1, E2 and a ground pad GP are connected and disconnected from two electrical output poles of a system configured to deliver electrical energy to a living body. The said two electrical output poles are identified as + and −. Each step of the sequence occurs in one of the time periods td1, td2, td3, td4, td5, td6, td7, td8, and td9. During the sequence, an electrical output signal is delivered to the electrical output poles + and −. A parameter Vd characterizing the electric output signal during the time periods of the sequence is plotted as line 3090 over the time-axis 3080. For example, the parameter Vd can give the level of the electrical output signal. For example, the parameter Vd can give the difference in potential between the two output poles + and −. The sequence of is configured to control measurements T1 and T2 to within respective target ranges of values over time. For example, T1 can be a measurement of temperature at electrode E1. For example, T2 can be a measurement of temperature at electrode E2. Measurement T1 is plotted during steps of the sequence as line 3091 over time-axis 3081. The lower bound of the target range for measurement T1 is plotted during the steps of the sequence as dotted line 3101 over time-axis 3081. The upper bound of the target range for measurement T1 is plotted during the steps of the sequence as dotted line 3111 over time-axis 3081. Measurement T2 is plotted during steps of the sequence as line 3092 over time-axis 3082. The lower bound of the target range for measurement T2 is plotted during the steps of the sequence as dotted line 3102 over time-axis 3082. The upper bound of the target range for measurement T2 is plotted during the steps of the sequence as dotted line 3112 over time-axis 3082. In one more specific example, the sequence can be produced by the system shown in FIG. 1. In another more specific example, the sequence can be produced by the system shown in FIG. 7B. In another more specific example, the sequence can be produced by the system shown in FIG. 8B. In another more specific example, the sequence can be produced by the system shown in FIG. 28.

Before the first step of the sequence, before time period td1, the measurements T1 and T2 are at their initial values. In the first step of the sequence, during time period td1, the ground pad GP is not connected to any output pole, electrode E1 is connected to output pole +, and electrode E2 is connected to output pole −. The output signal level Vd is increased to provide electrical energy configured to increase the measurements T1 and T2 toward their respective target ranges. Then, the output signal level Vd is decreased to prevent measurement T2 from exceeding the upper limit of its target range. During time period td1, the measurement T2 increases and achieves values within its target range, whereas the measurement T1 increases toward its target range, but T1 still remains outside its target range. In the next step, during time period td2, the ground pad GP is connected to output pole −, electrode E1 is connected to output pole +, electrode E2 is not connected to any output pole, and the output level Vd is decreased. This step is configured to deliver electrical energy to electrode E1 for the purpose of increasing the measurement T1 to within its target range, while limiting energy delivery to electrode E2 that might cause measurement T2 to rise above the upper bound of its target range. During time period td2, the measurement T2 decreases slightly but stays within its target range; and the measurement T1 increases to within its target range. In the next step, during time period td3, the ground pad GP is not connected to any output pole, electrode E1 is connected to output pole +, and electrode E2 is connected to output pole −. The output signal level Vd is decreased slightly to maintain measurements T1 and T2 within their respective target ranges. An advantage of this step is that electrical current is directed in the space between electrodes E1 and E2, rather than in the direction of the ground pad GP. In the next step, during time period td4, the ground pad GP is not connected to any output pole, electrode E1 is connected to output pole +, and electrode E2 is connected to output pole −. This repeats the connections of time period td3. The output signal level Vd is increased slightly to moderate a decline in the measurement T1, while still maintaining measurements T1 and T2 within their respective target ranges. During time period td4, measurement T2 approaches the upper bound of its target range, and measurement T1 approaches the lower bound of its target range. In the next step, during time period td5, the ground pad GP is connected to output pole −, electrode E1 is connected to output pole +, electrode E2 is not connected to any output pole, and the output level Vd is maintained at a constant value. The connections and output level Vd in this step are configured to increase the value of T1 away from the lower bound of its target range and toward the center of its target range. The connections in this step are also configured to reduce the value of T2 away from the upper bound of its target range and toward the center of its target range. Near the end of time period td5 and the beginning of time period td6, the target range for measurement T2 is shifted to subsume higher values, as indicated by the dotted lines 3102 and 3112. At the beginning of time period td6, the measurement T1 is within its target range, and measurement T2 is below its target range. In the next step, during time period td6, the ground pad GP is connected to output pole −, electrode E2 is connected to output pole +, electrode E1 is not connected to any output pole, and the output level Vd is increased to apply more energy to electrode E1 and to increase measurement T1 toward its target range. By the beginning of time period td7, the measurement T1 is approaching the lower bound of its target range, and measurement T2 remains below the lower bound of its target range. In the next step, during time period td7, the ground pad GP is not connected to any output pole, electrode E1 is connected to output pole −, electrode E2 is not connected to output pole +, and the output level Vd is decreased to moderate delivery of energy to electrodes E1 and E2, and thus, to hold measurement T1 within its target range, and to increase measurement T2 to within its target range. By the end of time period td7, the measurement T1 is approaching the upper bound of its target range. In the next step, during time period td8, the ground pad GP is connected to output pole −, electrode E2 is connected to output pole +, electrode E1 is not connected to any output pole, and the output level Vd is held constant to hold measurement T2 within its target range. Since electrode E1 is disconnected from all output poles, measurement T1 toward the lower bound of its target range. In the next step, during time period td9, the ground pad GP is not connected to any output pole, electrode E1 is connected to output pole −, electrode E2 is connected to output pole +, and the output level Vd is held constant to hold measurements T1 and T2 within their respective target ranges.

One advantage of the sequence presented in FIG. 32 is that it contains at least one step in which the electrodes E1 and E2 are connected to opposite poles of the power supply, with the ground pad GP disconnected from all poles; and it contains at least one other step in which one electrodes is connected to a different output pole than the ground pad GP is connected to. Another advantage of the example presented in FIG. 32 is that the measurements T1 and T2 are brought within their target ranges, and the sequence contains at least one step in which electrical current flows between electrodes E1 and E2.

In one example of the system presented in FIG. 32, the output poles of the system can include a radiofrequency output pole. In one example, the output poles of the system can be connected to a radiofrequency power supply. In another example, the output poles of the system can be connected to a pulsed radiofrequency power supply. In one example, the sequence can be automated. In another example, the steps in the sequence can occur rapidly relative to the physical dynamics of measurements that are being controlled. In another example, the steps of the sequence can each last less than 100 milliseconds. In another example, the steps of the sequence can each last less than 1 second. In another example, the steps of the sequence can each last less than 3 seconds. In another example, the steps of the sequence can each less than 3 seconds. In one example, the total duration of the switching sequence can up to 10 seconds. In another example, the total duration of the switching sequence can up to 30 seconds. In another example, the total duration of the switching sequence can up to 60 seconds. In another example, the total duration of the switching sequence can up to 120 seconds. In another example, the total duration of the switching sequence can up to 150 seconds. In another example, the total duration of the switching sequence can up to 180 seconds. In another example, the total duration of the switching sequence can up to 600 seconds. In another example, the total duration of the switching sequence can up to 1800 seconds. In another example, the total duration of the switching sequence can be greater than 1800 seconds.

In another example, the duration of each step in the sequence can be variable. In another example, the duration of each step in the sequence can be adjusted for the purpose of bringing measurements to within their respective target ranges. In another example, the duration of each step in the sequence can be adjusted for the purpose of controlling the average electrical output to an electrode over a time window greater than one step in the sequence. In another example, the duration of each step in the sequence can be adjusted for the purpose of controlling the average electrical output to a group of electrodes over a time window greater than one step in the sequence. In another example, the duration of each step in the sequence can be different for each step. In another example, the duration of each step in the sequence can have the same value as the duration of all other steps in the sequence.

In another example, the sequence shown in FIG. 32 can continue beyond the steps shown and can include steps which contain additional type of connections from the output poles +, − to the electrodes E1, E2 and ground pad GP. In another example, the sequence of connection from output poles to electrodes and the ground pad can differ from the specific sequence shown in FIG. 32. For example, the number of steps in the sequence can be different that the number shown in FIG. 32. For example, the number of steps in the sequence can be configured to a control objective and/or a clinical objective. For example, the identity of the connections in each step can be different than the identities shown in FIG. 32. For example, the identity of the connections in each step can be configured to achieve a control and/or clinical objective.

The example shown in FIG. 32 can be generalized such that the number of electrodes N and can be any number that is at least two N≧2, and the number of measurements can be the same number N. In another example, the number of steps in the sequence can be any number greater than one. In another example, a sequence can contains hundreds of steps. In another example, a sequence can contains thousands of steps. In another example, the sequence can consist of repeated configurations of connection from the output poles to the electrodes and/or ground pad. In another example, the sequence can be periodic in its steps. In another example, the number of output poles in the sequence can be any number that is at least 2. For example, three output poles can be used.

In another example, the number of electrodes E1, . . . , EN and the number of measurements T1, . . . , TN are both a number N that is at least two, and the sequence contains at least one step in which the ground pad GP is disconnected from all electrical output poles and at least two electrodes are connected to opposite electrical output poles. For example, the number of electrodes and number of measurements can both be N=3. For example, the number of electrodes and number of measurements can both be N=4. For example, the number of electrodes and number of measurements can both be N=5. For example, the number of electrodes and number of measurements can both be N=6. For example, the number of electrodes and number of measurements can both be a number greater than six.

In another example, the number of electrical output poles can be a number that is at least two. For example, the number of electrical output poles can be three. For example, the number of electrical output poles can be four. For example, the number of electrical output poles can be five. For example, the number of electrical output poles can be six. For example, the number of electrical output poles can be greater than six. For example, the number of electrical output poles can be equal to the number of electrodes. It is understood that for examples where the number of output poles is greater than two, there can be more than output electrical output signal parameter Vd.

In another example, the electrical output signal parameter Vd can have a fixed value throughout all steps of the sequence. In another example, the electrical output signal parameter Vd can be varied during the sequence. In another example, the electrical output signal parameter Vd can be a voltage. In another example, the electrical output signal parameter Vd can be a current. In another example, the electrical output signal parameter Vd can be a power. In another example, the electrical output signal parameter Vd can be the amplitude of a radiofrequency signal.

It is understood that some measurements increase with the application of electrical energy, and that other measurements decrease with the application of electrical energy. It is understood that some measurements, such as an impedance, have a non-linear relationship between the applied energy and the direction of change. It is understood that a measurement that decreases with the application of energy can be converted into a measurement that increase with the application of energy by inverting the sign of the first said measurement. It is understood that a measurement that has a non-linear or time-varying relationship to the application energy can be converted into a measurement that increase with the application of energy by applying a properly configured mathematical function to the first said measurement.

In one example, the measurements T1 and T2 include a temperature. In another example, the measurements T1 and T2 are the temperatures of the electrodes E1 and E2, respectively. In another example, the measurements T1 and T2 are associated with electrodes E1 and E2, respectively. In another example, the measurements T1 and T2 include a measurement from a remote temperature probe. In another example, the measurements T1 and T2 include an impedance. In another example, the measurements T1 and T2 are a function of impedances associated with electrodes E1 and E2, respectively. In another example, the measurement T1 is the impedance between the electrode E1 and the ground pad GP. In another example, the measurement T2 is the impedance between the electrode E2 and the ground pad GP. In another example, the measurements T1 and T2 include a function of an electrical parameter over a duration of time that includes at least two steps of the sequence. In another example, the measurements T1 and T2 include a function of an electrical parameter over a duration of time that is configured to match the rate of a physical process within the tissue into which electrodes E1 and/or E2 are situated. For example, the time duration can be 100 milliseconds. For example, the time duration can be 200 milliseconds. For example, the time duration can be 300 milliseconds. For example, the time duration can be 500 milliseconds. For example, the time duration can be 1000 milliseconds. For example, the time duration can be a value less than 100 millisecond. For example, the time duration can be a value greater than 1000 milliseconds. In another example, the measurements T1 and T2 include the time-average of an electrical parameter over at least two steps of the sequence. In another example, the measurements T1 and T2 include the time-average of a power over at least two steps of the sequence. In another example, the measurements T1 and T2 include the time-average of a current over at least two steps of the sequence. In another example, the measurements T1 and T2 include the time-average of a voltage over at least two steps of the sequence. In another example, the measurement T1 is a function of the electrical output delivered to electrode E1 over a time period that includes at least two steps in the sequence. In another example, the measurement T1 is the average power delivered to electrode 1 over a number of steps in the sequence, where that number is at least two. In another example, the measurement T1 is the average current delivered to electrode 1 over a number of steps in the sequence, where that number is at least two. In another example, the measurement T1 is the average voltage delivered to electrode 1 over a number of steps in the sequence, where that number is at least two. In another example, the measurement T1 is the average power delivered to electrode 1 over one period of a periodic sequence of steps, where each period includes at least two steps. In another example, the measurement T2 is a function of the electrical output delivered to electrode E2 over a time period that includes at least two steps in the sequence. In another example, the measurement T2 is the average power delivered to electrode 2 over a number of steps in the sequence, where that number is at least two. In another example, the measurement T2 is the average current delivered to electrode 2 over a number of steps in the sequence, where that number is at least two. In another example, the measurement T2 is the average voltage delivered to electrode 2 over a number of steps in the sequence, where that number is at least two. In another example, the measurement T2 is the average power delivered to electrode 2 over one period of a periodic sequence of steps, where each period includes at least two steps.

In another example, the upper and lower bounds of target ranges for each measurement, i.e. 3101 and 3111 for measurement T1, and 3102 and 3112 for measurement T2, can vary arbitrarily over the course of the sequence. For example, either the upper and lower bounds of the target range for a measurement can both move upward or both move downward at any time during a sequence. For example, the upper and lower bound of the target range for a measurement can change independently of the other bound. For example, the target range can become more narrow or become wider during a sequence.

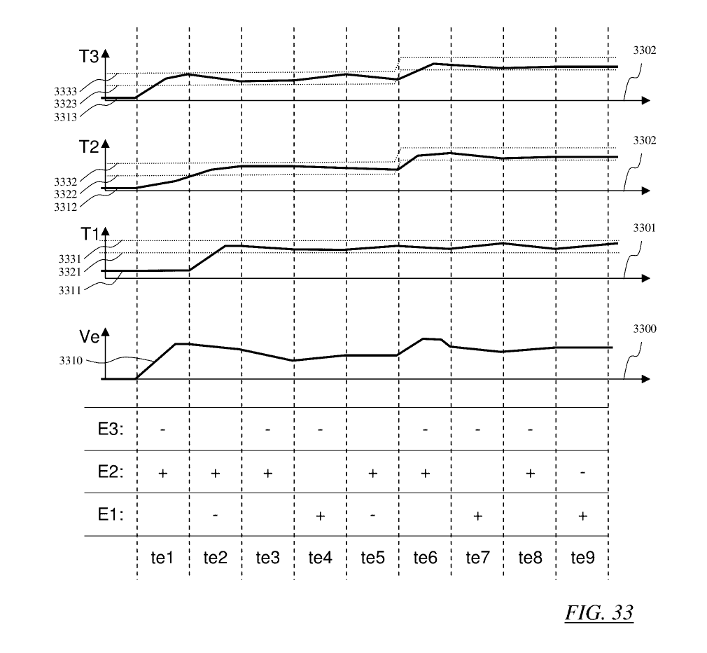

Referring now to FIG. 33, in accordance with one example of the present invention, a sequence is presented in which three electrodes E1, E2, and E3 are connected and disconnected from two electrical output poles of a system configured to deliver electrical energy to a living body. The said two electrical output poles are identified as + and −. Each step of the sequence occurs in one of the time periods te1, te2, te3, te4, te5, te6, te7, te8, and te9. During the sequence, an electrical output signal is delivered to the electrical output poles + and −. A parameter Ve characterizing the electric output signal during the time periods of the sequence is plotted as line 3310 over the time-axis 3300. For example, the parameter Ve can give the level of the electrical output signal. For example, the parameter Ve can give the difference in potential between the two output poles + and −. The sequence of is configured to control measurements T1, T2, and T3 to within respective target ranges of values over time. For example, T1 can be a measurement of temperature at electrode E1, T2 can be a measurement of temperature at electrode E2, and T3 can be a measurement of temperature at electrode E3. Measurement T1 is plotted during steps of the sequence as line 3311 over time-axis 3301. The lower bound of the target range for measurement T1 is plotted during the steps of the sequence as dotted line 3321 over time-axis 3301. The upper bound of the target range for measurement T1 is plotted during the steps of the sequence as dotted line 3331 over time-axis 3301. Measurement T2 is plotted during steps of the sequence as line 3311 over time-axis 3301. The lower bound of the target range for measurement T2 is plotted during the steps of the sequence as dotted line 3322 over time-axis 3302. The upper bound of the target range for measurement T2 is plotted during the steps of the sequence as dotted line 3332 over time-axis 3302. Measurement T3 is plotted during steps of the sequence as line 3313 over time-axis 3303. The lower bound of the target range for measurement T3 is plotted during the steps of the sequence as dotted line 3323 over time-axis 3303. The upper bound of the target range for measurement T3 is plotted during the steps of the sequence as dotted line 3333 over time-axis 3303. In one more specific example, the sequence can be produced by the system shown in FIG. 1. In another more specific example, the sequence can be produced by the system shown in FIG. 7A. In another more specific example, the sequence can be produced by the system shown in FIG. 8A. In another more specific example, the sequence can be produced by the system shown in FIG. 28.

Before the first step of the sequence, before time period te1, the measurements T1, T2, and T3 are at their initial values. In the first step of the sequence, during time period te1, electrode E1 is not connected to any output pole, electrode E2 is connected to output pole +, and electrode E3 is connected to output pole −. At first, the output signal level Ve is increased to provide electrical energy to electrodes E2 and E3 for the purpose of increasing the measurements T2 and T3 toward their respective target ranges. Then, the output signal level Ve is held at a constant value to prevent measurement T3 from exceeding the upper limit of its target range. During time period te1, the measurement T3 increases and achieves values within its target range, whereas the measurement T2 increases toward its target range, but T2 still remains below its target range. In the next step of the sequence, during time period te2, electrode E1 is connected to output pole −, electrode E2 is connected to output pole +, electrode E3 is not connected to any output pole, so that energy is applied to electrode E1 and E2 for the purpose of bringing measurements T1 and T2 to values within their respective target ranges. Over the time period te2, T1 increases to a value within its target range, T2 increases above the center of its target range, and T3 falls below the center of its target range. The output level Ve is decreased slightly over duration te2 to maintain measurements T1 and T2 near the center of their respective target ranges. In the next step of the sequence, during time period te3, electrode E1 is not connected to any output pole, electrode E2 is connected to output pole +, electrode E3 is connected to output pole −. The output level Ve is decreased to moderate the power delivered to electrodes E2 since measurement T2 is above the center of its target range. During time period te3, measurements T2 and T3 remain in their target ranges, and T1 declines toward the lower bound of its target range since no substantial energy is being delivered to E1. In the next step of the sequence, during time period te4, electrode E1 is connected to output pole +, electrode E2 is not connected to any output pole, electrode E3 is connected to output pole −, and the output level Ve is increased slightly to apply energy to electrodes E1 and E3. During time period te4, the measurement T1 is roughly level, the measurement T2 declines, and the measurement T3 increases toward the upper bound of its target range. In the next step of the sequence, during time period te5, electrode E1 is connected to output pole −, electrode E2 is connected to output pole +, electrode E3 is not connected to any output pole, and the output level Ve is substantially constant to apply a constant rate of energy to electrodes E1 and E2. During time period te5, measurement T1 increases toward the center of its target range, measurement T2 decreases, and measurement T3 decreases. Near the end of time period te5, the target ranges for T2 and T3 both increase to cover a higher range of measurement values. At the beginning of time period te6, the measurements T2 and T3 are below their target ranges. In the next step of the sequence, during time period te6, electrode E1 is not connected to any output pole, electrode E2 is connected to output pole +, electrode E3 is connected to output pole −, and the output level Ve is increased and then decreased to raise the measurements T2 and T3 to within their respective target ranges, while reducing the degree to which T2 and T3 overshoot the central values of their respective target ranges. During time period te6, the measurement T1 declines since energy is not being delivered to electrode E1. In the next step of the sequence, during time period te7, electrode E1 is connected to output pole +, electrode E2 is not connected to any output pole, electrode E3 is connected to output pole − to apply energy to electrodes E1 and E3. Over time period te7, the measurement T1 increases above the central region of its target range, so the output level Ve is decreased slightly to reduce the degree to which T1 overshoots said central region. This reduction in output level causes T3 to decline below the central region of its target range. Since electrical energy is not applied to electrode E2, measurement T2 declines below the central region of its target range. To counteract the decline in measurement T2 and T3, the next step of the control sequence is initiated. In the next step of the sequence, during time period te8, electrode E1 is not connected to any output pole, electrode E2 is connected to output pole +, electrode E3 is connected to output pole − to deliver energy to electrodes E2 and E3 and, thus, to counteract the decline in measurement T2 and T3. The output level Ve is increased to increase the energy delivered to electrode E2 as compared with the last step of the sequence. During time period te8, the measurements T2 and T3 are held at substantially constant values within their target ranges, and T1 decreases within its target range. In the next step of the sequence, during time period te9, electrode E1 is connected to output pole +, electrode E2 is connected to output pole −, electrode E3 is not connected to any output pole, and the output level Ve is substantially constant to apply a constant rate of energy to electrodes E1 and E2. During time period te9, the measurement T1 increases toward the upper bound of its target range, the measurement T2 has a roughly constant value within its target range, and the measurement T3 has a roughly constant value within its target range.

One advantage of the sequence presented in FIG. 33 is that measurements T1, T2, and T3 associated with electrodes T1, T2, and T3, respectively, are controlled at the same time without the use of an additional reference structure, such as a ground pad. Another advantage of the control sequence provided in FIG. 33 is that electrical energy is delivered only to electrodes for which a control objective is targeted. Another advantage of the control sequence provided in FIG. 33 is that electrical energy is delivered to electrodes E1, E2, and E3 a pairwise, bipolar manner in each step of the sequence. Another advantage of the control sequence provided in FIG. 33 is that the electrodes E1, E2, and E3 can be placed in biological tissue in a geometric pattern configured to direct electrical energy to the regions between pairs of electrodes.

In one example of the system presented in FIG. 33, the output poles of the system can include a radiofrequency output pole. In one example, the output poles of the system can be connected to a radiofrequency power supply. In another example, the output poles of the system can be connected to a pulsed radiofrequency power supply. In one example, the sequence can be automated. In another example, the steps in the sequence can occur rapidly relative to the physical dynamics of measurements that are being controlled. In another example, the steps of the sequence can each last less than 100 milliseconds. In another example, the steps of the sequence can each last less than 1 second. In another example, the steps of the sequence can each last less than 3 seconds. In another example, the steps of the sequence can each less than 3 seconds. In one example, the total duration of the switching sequence can up to 10 seconds. In another example, the total duration of the switching sequence can up to 30 seconds. In another example, the total duration of the switching sequence can up to 60 seconds. In another example, the total duration of the switching sequence can up to 120 seconds. In another example, the total duration of the switching sequence can up to 150 seconds. In another example, the total duration of the switching sequence can up to 180 seconds. In another example, the total duration of the switching sequence can up to 600 seconds. In another example, the total duration of the switching sequence can up to 1800 seconds. In another example, the total duration of the switching sequence can be greater than 1800 seconds.

In another example, the duration of each step in the sequence can be variable. In another example, the duration of each step in the sequence can be adjusted for the purpose of bringing measurements to within their respective target ranges. In another example, the duration of each step in the sequence can be adjusted for the purpose of controlling the average electrical output to an electrode over a time window greater than one step in the sequence. In another example, the duration of each step in the sequence can be adjusted for the purpose of controlling the average electrical output to a group of electrodes over a time window greater than one step in the sequence. In another example, the duration of each step in the sequence can be different for each step. In another example, the duration of each step in the sequence can have the same value as the duration of all other steps in the sequence.

In another example, the sequence shown in FIG. 33 can continue beyond the steps shown and can include steps which contain additional type of connections from the output poles +, − to the electrodes E1, E2, and E3. In another example, the sequence of connection from output poles to electrodes can differ from the specific sequence shown in FIG. 33. For example, the number of steps in the sequence can be different that the number shown in FIG. 33. For example, the number of steps in the sequence can be configured to a control objective and/or a clinical objective. For example, the identity of the connections in each step can be different than the identities shown in FIG. 33. For example, the identity of the connections in each step can be configured to achieve a control and/or clinical objective.

The example shown in FIG. 33 can be generalized such that the number of electrodes N and can be any number that is at least three N≧3, and the number of measurements can be the same number N. In another example, the number of steps in the sequence can be any number that is at least three. In another example, a sequence can contains hundreds of steps. In another example, a sequence can contains thousands of steps. In another example, the sequence can consist of repeated configurations of connection from the output poles to the electrodes and/or ground pad. In another example, the sequence can be periodic in its steps.

In another example, the number of electrodes E1, . . . , EN and the number of measurements T1, . . . , TN are both a number N that is at least N=3. For example, the number of electrodes and number of measurements can both be N=3. For example, the number of electrodes and number of measurements can both be N=4. For example, the number of electrodes and number of measurements can both be N=5. For example, the number of electrodes and number of measurements can both be N=6. For example, the number of electrodes and number of measurements can both be a number greater than six.

In another example, the number of electrical output poles can be a number that is at least two. For example, the number of electrical output poles can be three. For example, the number of electrical output poles can be four. For example, the number of electrical output poles can be five. For example, the number of electrical output poles can be six. For example, the number of electrical output poles can be greater than six. For example, the number of electrical output poles can be equal to the number of electrodes. It is understood that for examples where the number of output poles is greater than two, there can be more than output electrical output signal parameter Ve.

In another example, the electrical output signal parameter Ve can have a fixed value throughout all steps of the sequence. In another example, the electrical output signal parameter Ve can be varied during the sequence. In another example, the electrical output signal parameter Ve can be a voltage. In another example, the electrical output signal parameter Ve can be a current. In another example, the electrical output signal parameter Ve can be a power. In another example, the electrical output signal parameter Ve can be the amplitude of a radiofrequency signal.

It is understood that some measurements increase with the application of electrical energy, and that other measurements decrease with the application of electrical energy. It is understood that some measurements, such as an impedance, have a non-linear relationship between the applied energy and the direction of change. It is understood that a measurement that decreases with the application of energy can be converted into a measurement that increase with the application of energy by inverting the sign of the first said measurement. It is understood that a measurement that has a non-linear or time-varying relationship to the application energy can be converted into a measurement that increase with the application of energy by applying a properly configured mathematical function to the first said measurement.

In one example, the measurements T1, T2, and T3 include a temperature. In another example, the measurements T1, T2, and T3 are the temperatures of the electrodes E1, E2, and E3, respectively. In another example, the measurements T1, T2, and T3 are associated with electrodes E1, E2, and E3, respectively. In another example, the measurements T1, T2, and T3 include a measurement from a remote temperature probe. In another example, the measurements T1, T2, and T3 include an impedance. In another example, the measurements T1, T2, and T3 are a function of impedances associated with electrodes E1, E2, and E3, respectively. In another example, the measurement T1 is the impedance to current flow from electrode E1 to both electrodes E2 and E3. In another example, the measurement T2 is the impedance to current flow from electrode E2 to both electrodes E1 and E3. In another example, the measurement T3 is the impedance to current flow from electrode E3 to both electrodes E2 and E1. In another example, the measurement T1 is the impedance measured when electrode E1 is connected to one output pole and all other electrodes are connected to a different output pole. In another example, the measurement T2 is the impedance measured when electrode E2 is connected to one output pole and all other electrodes are connected to a different output pole. In another example, the measurement T3 is the impedance measured when electrode E3 is connected to one output pole and all other electrodes are connected to a different output pole. In another example, the measurements T1, T2, and T3 include a function of an electrical parameter over a duration of time that includes at least two steps of the sequence. In another example, the measurements T1, T2, and T3 include a function of an electrical parameter over a duration of time that is configured to match the rate of a physical process within the tissue into which electrodes E1, E2, and/or E3 are situated. For example, the time duration can be 100 milliseconds. For example, the time duration can be 200 milliseconds. For example, the time duration can be 300 milliseconds. For example, the time duration can be 500 milliseconds. For example, the time duration can be 1000 milliseconds. For example, the time duration can be a value less than 100 millisecond. For example, the time duration can be a value greater than 1000 milliseconds. In another example, the measurements T1, T2, and T3 include the time-average of a parameter that characterizes the electrical signal applied to output poles over a duration of time that is greater than the duration of one step in the sequence. In another example, the measurements T1, T2, and T3 include the time-average of the power delivered to one or more electrodes over at least two steps of the sequence. In another example, the measurements T1, T2, and T3 include the time-average of the current delivered to one or more electrodes over at least two steps of the sequence. In another example, the measurements T1, T2, and T3 include the RMS current delivered to one or more electrodes over at least two steps of the sequence. In another example, the measurements T1, T2, and T3 include the time-average of the voltage applied to an electrode over at least two steps of the sequence. In another example, the measurements T1, T2, and T3 include the RMS voltage delivered to one or more electrodes over at least two steps of the sequence.

In another example, the upper and lower bounds of target ranges for each measurement (i.e. 3321 and 3331 for measurement T1, 3322 and 3332 for measurement T2, and 3323 and 3333 for measurement T3) can vary arbitrarily over the course of the sequence. For example, either the upper and lower bounds of the target range for a measurement can both move upward or both move downward at any time during a sequence. For example, the upper and lower bound of the target range for a measurement can change independently of the other bound. For example, the target range can become more narrow or become wider during a sequence.

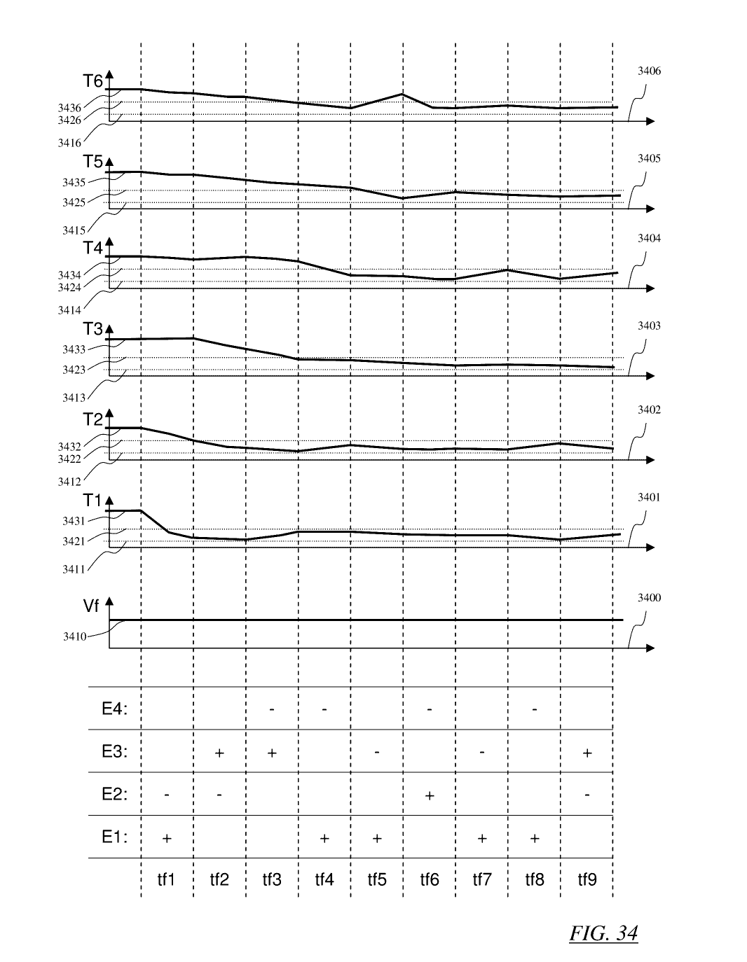

Referring now to FIG. 34, in accordance with one example of the present invention, a sequence is presented in which three electrodes E1, E2, and E3 are connected and disconnected from two electrical output poles of a system configured to deliver electrical energy to a living body. The said two electrical output poles are identified as + and −. Each step of the sequence occurs in one of the time periods tf1, tf2, tf3, tf4, tf5, tf6, tf7, tf8, and tf9. During the sequence, an electrical output signal is delivered to the electrical output poles + and −. A parameter Vf characterizing the electric output signal during the time periods of the sequence is plotted as line 3310 over the time-axis 3300. For example, the parameter Vf can give the level of the electrical output signal. For example, the parameter Vf can give the difference in potential between the two output poles + and −. The sequence of is configured to control measurements T1, T2, T3, T4, T5, and T6 to within respective target ranges of values over time. In the example shown, the measurements tend to decrease with the application of electrical energy. For example, T1 can be a measurement of impedance between electrodes E1 and E2, T2 can be a measurement of impedance between electrodes E2 and E3, T3 can be a measurement of impedance between electrodes E3 and E4, T4 can be a measurement of impedance between electrodes E4 and E1, T5 can be a measurement of impedance between electrodes E1 and E3, T6 can be a measurement of impedance between electrodes E2 and E4. In another example, T1, T2, T3, and T4 can be inverted measurements of the temperatures at electrodes E1, E2, E3, and E4, respectively; T5 can be the impedance between electrodes E1 and E3; and T6 can be the impedance between electrodes E2 and E4.

Measurement T1 is plotted during steps of the sequence as line 3431 over time-axis 3401. The lower bound of the target range for measurement T1 is plotted during the steps of the sequence as dotted line 3411 over time-axis 3401. The upper bound of the target range for measurement T1 is plotted during the steps of the sequence as dotted line 3421 over time-axis 3401. Measurement T2 is plotted during steps of the sequence as line 3432 over time-axis 3402. The lower bound of the target range for measurement T2 is plotted during the steps of the sequence as dotted line 3412 over time-axis 3402. The upper bound of the target range for measurement T2 is plotted during the steps of the sequence as dotted line 3422 over time-axis 3402. Measurement T3 is plotted during steps of the sequence as line 3433 over time-axis 3403. The lower bound of the target range for measurement T3 is plotted during the steps of the sequence as dotted line 3413 over time-axis 3403. The upper bound of the target range for measurement T3 is plotted during the steps of the sequence as dotted line 3423 over time-axis 3403. Measurement T4 is plotted during steps of the sequence as line 3434 over time-axis 3404. The lower bound of the target range for measurement T4 is plotted during the steps of the sequence as dotted line 3414 over time-axis 3404. The upper bound of the target range for measurement T4 is plotted during the steps of the sequence as dotted line 3424 over time-axis 3404. Measurement T5 is plotted during steps of the sequence as line 3435 over time-axis 3405. The lower bound of the target range for measurement T5 is plotted during the steps of the sequence as dotted line 3415 over time-axis 3405. The upper bound of the target range for measurement T5 is plotted during the steps of the sequence as dotted line 3425 over time-axis 3405. Measurement T6 is plotted during steps of the sequence as line 3436 over time-axis 3406. The lower bound of the target range for measurement T6 is plotted during the steps of the sequence as dotted line 3416 over time-axis 3406. The upper bound of the target range for measurement T6 is plotted during the steps of the sequence as dotted line 3426 over time-axis 3406.

In one more specific example, the sequence can be produced by the system shown in FIG. 1. In another more specific example, the sequence can be produced by the system shown in FIG. 7A. In another more specific example, the sequence can be produced by the system shown in FIG. 8A. In another more specific example, the sequence can be produced by the system shown in FIG. 28.

In the example sequence shown in FIG. 34, the output level Vf is held at a constant value for all steps tf1, tf2, tf3, tf4, tf5, tf6, tf7, tf8, tf9. Before the first step of the sequence, before time period tf1, the measurements T1, T2, T3, T4, T5, and T6 are at their initial values, outside their target ranges.

In the first step of the sequence, during time period tf1, electrode E1 is connected to output pole +, electrode E2 is connected to output pole −, and electrodes E3 and E4 are not connected to any output pole. During time period tf1, the measurement T1 decreases and achieves values within its target range; measurements T2, T4, T5, and T6 decrease slightly toward their respective target ranges; and measurement T3 is substantially unchanged.

In the next step of the sequence, during time period tf2, electrode E3 is connected to output pole +, electrode E2 is connected to output pole −, and electrodes E1 and E4 are not connected to any output pole. During time period tf2, the measurement T2 decreases and achieves values within its target range; measurements T1, T3, T5, and T6 decrease, with T1 staying within its target range; and measurement T4 increases slightly toward its initial value.

In the next step of the sequence, during time period tf3, electrode E3 is connected to output pole +, electrode E4 is connected to output pole −, and electrodes E1 and E2 are not connected to any output pole. During time period tf3, the measurement T3 decreases and achieves values within its target range; measurements T2, T4, T5, and T6 decrease, with T2 remaining in its target range; and measurement T1 increases slightly but stays within its target range.

In the next step of the sequence, during time period tf4, electrode E1 is connected to output pole +, electrode E4 is connected to output pole −, and electrodes E2 and E3 are not connected to any output pole. During time period tf4, the measurements T4 and T6 decrease and achieve values within their respective target ranges; measurements T1, T3, and T5 decrease slightly, with T1 and T3 remaining in their respective target ranges; and measurement T2 increases but stays within its target range.

In the next step of the sequence, during time period tf5, electrode E1 is connected to output pole +, electrode E3 is connected to output pole −, and electrodes E2 and E4 are not connected to any output pole. During time period tf5, the measurement T5 decreases and achieves values within its target range; measurements T1, T2, T3, and T4 decrease slightly and stay within their respective target ranges; and measurement T6 increases towards its initial value outside its target range.

In the next step of the sequence, during time period tf6, electrode E2 is connected to output pole +, electrode E4 is connected to output pole −, and electrodes E1 and E3 are not connected to any output pole. During time period tf6, the measurement T6 decreases and achieves values within its target range; measurements T1 and T2 are substantially unchanged within their respective target ranges; T3 and T4 decrease slightly, with T4 approaching the lower limit of its target range; and measurement T5 increases toward the upper limit of its target range.

In the next step of the sequence, during time period tf7, electrode E1 is connected to output pole +, electrode E3 is connected to output pole −, and electrodes E2 and E4 are not connected to any output pole. During time period tf7, the measurement T5 decreases from the upper limit of its target range to a more central values within its target range; measurements T1, T2, T3, and T6 are maintained at values near the centers of their respective target ranges; and measurement T4 increases toward the upper limit of its target range.

In the next step of the sequence, during time period tf8, electrode E1 is connected to output pole +, electrode E4 is connected to output pole −, and electrodes E2 and E3 are not connected to any output pole. During time period tf8, the measurement T4 decreases from the upper limit of its target range toward the bottom of its target range; measurements T3, T5, and T6 are maintained at values within their respective target ranges; measurement T1 decreases to near the bottom limit of its target range; and measurement T2 increases toward the upper limit of its target range.

In the next step of the sequence, during time period tf9, electrode E3 is connected to output pole +, electrode E2 is connected to output pole −, and electrodes E1 and E4 are not connected to any output pole. During time period tf9, the measurement T2 decreases from the upper limit of its target range toward the bottom of its target range; measurements T3, T5, and T6 are maintained at values within their respective target ranges; and measurements T1 and T4 increases toward the upper limits of their respective target ranges.

One advantage of the sequence presented in FIG. 34 is that measurements T1, T2, T3, T4, T5, and T6 associated with electrodes E1, E2, E3, and E4 are controlled at the same time without the use of an additional reference structure, such as a ground pad. Another advantage of the control sequence provided in FIG. 34 is that the number of parameters being controlled at the same time exceeds the number of electrodes, without the use of an additional reference structure, such as a ground pad. Another advantage of the control sequence provided in FIG. 34 is that electrical energy is delivered only to electrodes for which a control objective is targeted. Another advantage of the control sequence provided in FIG. 34 is that electrical energy is delivered to electrodes E1, E2, E3, and E4 in a pairwise, bipolar manner in each step of the sequence. Another advantage of the control sequence provided in FIG. 34 is that the electrodes E1, E2, E3, and E4 can be placed in biological tissue in a geometric pattern configured to direct electrical energy to the regions between pairs of electrodes. Another advantage of the control sequence provided in FIG. 34 is that the electrodes E1, E2, E3, and E4 can be placed in biological tissue to control simultaneously a physical parameter associated with the tissue between each pair of electrodes, such as the impedance between each pair of electrode. Another advantage of the control sequence provided in FIG. 34 is that the frequency with which each group of electrode is energized is varied to control the measurements T1, T2, T3, T4, T5, and T6 at the same time (note that group E1-E3 is energized in steps tf5 and tf7; group E1-E4 is energized in steps tf4 and tf8; and group E2-E3 is energized in steps tf2 and tf9).

In one example of the system presented in FIG. 34, the output poles of the system can include a radiofrequency output pole. In one example, the output poles of the system can be connected to a radiofrequency power supply. In another example, the output poles of the system can be connected to a pulsed radiofrequency power supply. In one example, the sequence can be automated. In another example, the steps in the sequence can occur rapidly relative to the physical dynamics of measurements that are being controlled. In another example, the steps of the sequence can each last for a duration less than 100 milliseconds. In another example, the steps of the sequence can each last 100 milliseconds. In another example, the steps of the sequence can each last 200 milliseconds. In another example, the steps of the sequence can each last 300 milliseconds. In another example, the steps of the sequence can each last 400 milliseconds. In another example, the steps of the sequence can each last 500 milliseconds. In another example, the steps of the sequence can each last 600 milliseconds. In another example, the steps of the sequence can each last 700 milliseconds. In another example, the steps of the sequence can each last 800 milliseconds. In another example, the steps of the sequence can each last 900 milliseconds. In another example, the steps of the sequence can each last 1 second. In another example, the steps of the sequence can each last less than 1 second. In another example, the steps of the sequence can each last less than 3 seconds. In another example, the steps of the sequence can each less than 15 seconds. In another example, the steps of the sequence can each be greater than 15 seconds. In another example, the duration of each step in the sequence can be variable. In another example, the duration of each step in the sequence can be adjusted for the purpose of bringing measurements to within their respective target ranges. In another example, the duration of each step in the sequence can be adjusted for the purpose of controlling the average electrical output to an electrode over a time window greater than one step in the sequence. In another example, the duration of each step in the sequence can be adjusted for the purpose of controlling the average electrical output to a group of electrodes over a time window greater than one step in the sequence. In another example, the duration of each step in the sequence can be different for each step. In another example, the duration of each step in the sequence can have the same value as the duration of all other steps in the sequence.

In one example, the total duration of the switching sequence can up to 10 seconds. In another example, the total duration of the switching sequence can up to 30 seconds. In another example, the total duration of the switching sequence can up to 60 seconds. In another example, the total duration of the switching sequence can up to 90 seconds. In another example, the total duration of the switching sequence can up to 120 seconds. In another example, the total duration of the switching sequence can up to 150 seconds. In another example, the total duration of the switching sequence can up to 180 seconds. In another example, the total duration of the switching sequence can up to 600 seconds. In another example, the total duration of the switching sequence can up to 1800 seconds. In another example, the total duration of the switching sequence can be greater than 1800 seconds.

In another example, the sequence shown in FIG. 34 can continue beyond the steps shown and can include steps which contain additional type of connections from the output poles +, − to the electrodes E1, E2, E3, and E4. In another example, the sequence of connection from output poles to electrodes can differ from the specific sequence shown in FIG. 34. For example, the number of steps in the sequence can be different that the number shown in FIG. 34. For example, the number of steps in the sequence can be configured to a control objective and/or a clinical objective. For example, the identity of the connections in each step can be different than the identities shown in FIG. 34. For example, the identity of the connections in each step can be configured to achieve a control and/or clinical objective.

The example shown in FIG. 34 can be generalized such that the number of electrodes N and can be any number that is at least three N≧3, and the number of measurements can be a number P greater than or equal to N. For example, the number of measurements can be equal to the number of pairs of electrodes P=N*(N−1)/2. In another example, the number of steps in the sequence can be any number that is at least three. In another example, a sequence can contains hundreds of steps. In another example, a sequence can contains thousands of steps. In another example, the sequence can consist of repeated configurations of connection from the output poles to the electrodes and/or ground pad. In another example, the sequence can be periodic in its steps.

In another example, the number of electrodes E1, . . . , EN is a number N that is at least 3, and the number of measurements T1, . . . , TP is a number P that is great than or equal to the number N. For example, the number of electrodes and number of measurements can both be N=P=3. For example, the number of electrodes and number of measurements can both be N=4. For example, the number of electrodes can be N=4, and the number of measurements can be P=6. For example, the number of electrodes and number of measurements can both be N=P=5. For example, the number of electrodes can be N=5, and the number of measurements can be P=10. For example, the number of electrodes and number of measurements can both be N=P=6. For example, the number of electrodes can be N=6, and the number of measurements can be P=15. For example, the number of electrodes and number of measurements can both be a number greater than six. For example, the number of electrodes can be a number greater than six, and the number of measurements can be a number than that number of electrodes.

In another example, the number of electrical output poles can be a number that is at least two. For example, the number of electrical output poles can be three. For example, the number of electrical output poles can be four. For example, the number of electrical output poles can be five. For example, the number of electrical output poles can be six. For example, the number of electrical output poles can be greater than six. For example, the number of electrical output poles can be equal to the number of electrodes. It is understood that for examples where the number of output poles is greater than two, there can be more than output electrical output signal parameter Vf.

In another example, the electrical output signal parameter Vf can have a fixed value throughout all steps of the sequence. In another example, the electrical output signal parameter Vf can be varied during the sequence. In another example, the electrical output signal parameter Vf can be a voltage. In another example, the electrical output signal parameter Vf can be a current. In another example, the electrical output signal parameter Vf can be a power. In another example, the electrical output signal parameter Vf can be the amplitude of a radiofrequency signal.

It is understood that some measurements increase with the application of electrical energy, and that other measurements decrease with the application of electrical energy. It is understood that some measurements, such as an impedance, have a non-linear relationship between the applied energy and the direction of change. For example, the impedance measured between an electrode and another structure can decrease as the application of energy increases the temperature of the tissue in which the electrode is placed, while that temperature is substantially below boiling; however, the said impedance can then increase with the application of energy if the temperature of said tissue exceeds boiling. It is understood that a measurement that decreases with the application of energy can be converted into a measurement that increases with the application of energy by inverting the sign of the first said measurement. It is understood that a measurement that has a non-linear or time-varying relationship to the application energy can be converted into a measurement that increase with the application of energy by applying a properly configured mathematical function to the first said measurement.

In one example, the measurements T1, T2, T3, T4, T5, and T6 include a temperature. In another example, the measurements T1, T2, T3, T4, T5, and T6 include a measurement from a remote temperature probe. In another example, the measurements T1, T2, T3, T4 are the temperatures of the electrodes E1, E2, E3, and E4, respectively, and T5 and T6 are temperatures of remote temperature probes. In another example, the measurements T1, T2, T3, T4, T5, and T6 are measurements from temperature probes place between each pair of electrodes E1, E2, E3, and E4. In another example, the measurements T1, T2, T3 and T4 are associated with electrodes E1, E2, E3 and E4, respectively. In another example, the measurements T1, T2, T3, T4, T5, and T6 include an impedance. In another example, the measurements T1, T2, T3, T4, T5, and T6 are a function of impedances associated with electrodes E1, E2, E3 and E4, respectively. In another example, the measurements T1, T2, T3, T4, T5, and T6 are the impedance to current flow between each pair of electrodes E1, E2, E3, and E4. In another example, each measurement T1, T2, T3, T4, T5, and T6 is the impedance to current flow from one electrode to at least one other electrode. In another example, the measurements T1, T2, T3, T4, T5, and T6 include the impedance measured when one electrode is attached to one output pole, and all other electrodes are connected to a different output pole. In another example, the measurements T1, T2, T3, T4, T5, and T6 include a function of an electrical parameter over a duration of time that includes at least two steps of the sequence. In another example, the measurements T1, T2, T3, T4, T5, and T6 include a function of an electrical parameter over a duration of time that is configured to match the rate of a physical process within the tissue into which electrodes E1, E2, E3 and/or E4 are situated. For example, the time duration can be 100 milliseconds. For example, the time duration can be 200 milliseconds. For example, the time duration can be 300 milliseconds. For example, the time duration can be 500 milliseconds. For example, the time duration can be 1000 milliseconds. For example, the time duration can be a value less than 100 millisecond. For example, the time duration can be a value greater than 1000 milliseconds. In another example, the measurements T1, T2, T3, T4, T5, and T6 include the time-average of a parameter that characterizes the electrical signal applied to output poles over a duration of time that is greater than the duration of one step in the sequence. In another example, the measurements T1, T2, T3, T4, T5, and T6 include the time-average of the power delivered to one or more electrodes over at least two steps of the sequence. In another example, the measurements T1, T2, T3, T4, T5, and T6 include the time-average of the current delivered to one or more electrodes over at least two steps of the sequence. In another example, the measurements T1, T2, T3, T4, T5, and T6 include the RMS current delivered to one or more electrodes over at least two steps of the sequence. In another example, the measurements T1, T2, T3, T4, T5, and T6 include the time-average of the voltage applied to an electrode over at least two steps of the sequence. In another example, the measurements T1, T2, T3, T4, T5, and T6 include the RMS voltage delivered to one or more electrodes over at least two steps of the sequence.

In another example, the upper and lower bounds of target ranges for each measurement (i.e. 3421 and 3431 for measurement T1, 3422 and 3432 for measurement T2, 3423 and 3433 for measurement T3, 3424 and 3434 for measurement T4, 3425 and 3435 for measurement T5, and 3426 and 3436 for measurement T6) can vary arbitrarily over the course of the sequence. For example, either the upper and lower bounds of the target range for a measurement can both move upward or both move downward at any time during a sequence. For example, the upper and lower bound of the target range for a measurement can change independently of the other bound. For example, the target range can become more narrow or become wider during a sequence.

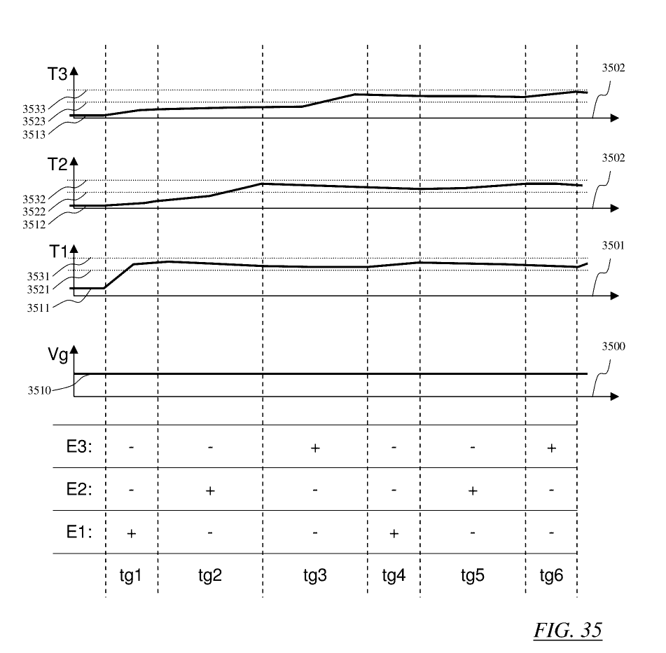

Referring now to FIG. 35, in accordance with one example of the present invention, a sequence is presented in which three electrodes E1, E2, and E3 are connected and disconnected from two electrical output poles of a system configured to deliver electrical energy to a living body. The said two electrical output poles are identified as + and −. Each step of the sequence occurs in one of the time periods tg1, tg2, tg3, tg4, tg5, and tg6. During the sequence, an electrical output signal is delivered to the electrical output poles + and −. A parameter Vg characterizing the electric output signal during the time periods of the sequence is plotted as line 3410 over the time-axis 3400. In this example, the parameter is held at a fixed value over the entire sequence. Measurements T1, T2, and T3 are associated with each electrode E1, E2, and E3, respectively. Measurements T1, T2, and T3 and their target bounds are depicted in FIG. 35 in a manner analogous to measures of the same names in FIG. 33. In the example shown in FIG. 35, the step tg2, tg3, and tg5 each have durations that are longer than steps tg1, tg3, and tg6. In the example shown in FIG. 35, in each step of the sequence, only one electrode is connected to pole +, and the other electrodes are connected to pole −. One advantage of the sequence presented in FIG. 35 is that measurements T1, T2, and T3 associated with electrodes T1, T2, and T3, respectively, are controlled at the same time without the use of an additional reference structure, such as a ground pad. Another advantage of the control sequence provided in FIG. 35 is that electrical energy is delivered only to electrodes for which a control objective is targeted. Another advantage of the control sequence provided in FIG. 35 is that electrical energy is delivered to electrodes E1, E2, and E3 such that, in each step, the return currents from one electrodes are distributed over more than one other electrode. Another advantage of the control sequence provided in FIG. 35 is that the electrodes E1, E2, and E3 can be placed in biological tissue in a geometric pattern configured to direct electrical energy to the regions between pairs of electrodes. It is understood that the example shown in FIG. 35 can be generalized to any number of electrodes that is greater than or equal to three.

Referring to the example sequences provided in FIGS. 32, 33, 34, and 35, in more specific examples of the provided sequences, a measurement can take one of a number of different forms. For example, a measurement can be a temperature. For example, a measurement can be the temperature measured at an electrode. For example, a measurement can be the temperature of tissue adjacent to an electrode. For example, a measurement can be the temperature of tissue nearby an electrode. For example, a measurement can be a temperature measured at a location remote of an electrode. For example, a measurement can be an impedance. For example, a measurement can be the impedance between an electrodes and a reference structure, such as a ground pad. For example, a measurement can be the impedance between an electrode and other electrodes that are placed in contact with the same body of tissue. For example, a measurement can be the impedance to electrical current flowing from one electrode to other electrodes. For example, a measurement can be an average power. For example, a measurement can be an average power over a duration of time. For example, a measurement can be an average power over a duration of time that includes more than one step in a sequence of connections, where said more than one steps include more than one configurations of connections between electrodes and output poles of an electrical generator. For example, a measurement can be a voltage. For example, a measurement can be a root-mean-square (RMS) voltage. For example, a measurement can be an average voltage over a duration of time that includes more than one step in a sequence of connections, where said more than one steps include more than one configurations of connections between electrodes and output poles of an electrical generator. For example, a measurement can be a current. For example, a measurement can be a root-mean-square (RMS) current. For example, a measurement can be an average current over a duration of time that includes more than one step in a sequence of connections, where said more than one steps include more than one configurations of connections between electrodes and output poles of an electrical generator. For example, a measurement can be the function of characteristics of the electrical signals applied to output poles of the generator. For example, a measurement can be the function of characteristics of the electrical signals applied to output poles of the generator over a duration of time that includes more than one step in a sequence of connections, where said more than one steps include more than one configurations of connections between electrodes and output poles of an electrical generator. For example, a measurement can be the output of probe that measures tissue water content. For example, a measurement can the output of probe that measures some physical property of biological tissue.

Referring to the example sequences provided in FIGS. 32, 33, 34, and 35, in more specific examples of the provided sequences, the target range for a measurement can be one or more intervals of measurement values. For example, the target range for a measurement can be a single contiguous set of measurement values. For example, the target range for a measurement can be range of measurement values including a gap. For example, the target range for a measurement can be two disjoint intervals of measurement values. For example, for a measurement that is a temperature, the target range can span less than 1° C. For example, for a measurement that is a temperature, the target range can span 1° C. For example, for a measurement that is a temperature, the target range can span 2° C. For example, for a measurement that is a temperature, the target range can span 4° C. For example, for a measurement that is a temperature, the target range can span 5° C. For example, for a measurement that is a temperature, the target range can span 10° C. For example, for a measurement that is a temperature, the target range can span 15° C. For example, for a measurement that is a temperature, the target range can span 20° C. For example, for a measurement that is a temperature, the target range can span 30° C. For example, for a measurement that is a temperature, the target range can span more than 30° C. For example, for a measurement that is an impedance, the target range can span less than 1 Ohms. For example, for a measurement that is an impedance, the target range can span 1 Ohms. For example, for a measurement that is an impedance, the target range can span 2 Ohms. For example, for a measurement that is an impedance, the target range can span 4 Ohms. For example, for a measurement that is an impedance, the target range can span 5 Ohms. For example, for a measurement that is an impedance, the target range can span 10 Ohms. For example, for a measurement that is an impedance, the target range can span 15 Ohms. For example, for a measurement that is an impedance, the target range can span 20 Ohms. For example, for a measurement that is an impedance, the target range can span 30 Ohms. For example, for a measurement that is an impedance, the target range can span more than 30 Ohms. For example, for a measurement that is a power, the target range can span less than 1 Watts. For example, for a measurement that is a power, the target range can span 1 Watts. For example, for a measurement that is a power, the target range can span 2 Watts. For example, for a measurement that is a power, the target range can span 4 Watts. For example, for a measurement that is a power, the target range can span 5 Watts. For example, for a measurement that is a power, the target range can span 10 Watts. For example, for a measurement that is a power, the target range can span 15 Watts. For example, for a measurement that is a power, the target range can span 20 Watts. For example, for a measurement that is a power, the target range can span 30 Watts. For example, for a measurement that is a power, the target range can span 40 Watts. For example, for a measurement that is a power, the target range can span 50 Watts. For example, for a measurement that is a power, the target range can span more than 50 Watts. For example, for a measurement that is a voltage, the target range can span less than 1 Volts. For example, for a measurement that is a voltage, the target range can span 1 Volts. For example, for a measurement that is a voltage, the target range can span 2 Volts. For example, for a measurement that is a voltage, the target range can span 4 Volts. For example, for a measurement that is a voltage, the target range can span 5 Volts. For example, for a measurement that is a voltage, the target range can span 10 Volts. For example, for a measurement that is a voltage, the target range can span 15 Volts. For example, for a measurement that is a voltage, the target range can span 20 Volts. For example, for a measurement that is a voltage, the target range can span 30 Volts. For example, for a measurement that is a voltage, the target range can span 40 Volts. For example, for a measurement that is a voltage, the target range can span 50 Volts. For example, for a measurement that is a voltage, the target range can span more than 50 Volts. For example, for a measurement that is a current, the target range can span less than 1 Milliamps. For example, for a measurement that is a current, the target range can span 1 Milliamps. For example, for a measurement that is a current, the target range can span 2 Milliamps. For example, for a measurement that is a current, the target range can span 4 Milliamps. For example, for a measurement that is a current, the target range can span 5 Milliamps. For example, for a measurement that is a current, the target range can span 10 Milliamps. For example, for a measurement that is a current, the target range can span 15 Milliamps. For example, for a measurement that is a current, the target range can span 20 Milliamps. For example, for a measurement that is a current, the target range can span 30 Milliamps. For example, for a measurement that is a current, the target range can span 40 Milliamps. For example, for a measurement that is a current, the target range can span 50 Milliamps. For example, for a measurement that is a current, the target range can span 100 Milliamps. For example, for a measurement that is a current, the target range can span 200 Milliamps. For example, for a measurement that is a current, the target range can span 300 Milliamps. For example, for a measurement that is a current, the target range can span 500 Milliamps. For example, for a measurement that is a current, the target range can span 1000 Milliamps. For example, for a measurement that is a current, the target range can span 1500 Milliamps. For example, for a measurement that is a current, the target range can span 2000 Milliamps. For example, for a measurement that is a current, the target range can span more than 2000 Milliamps.

Referring to the example sequences provided in FIGS. 32, 33, 34, and 35, in more specific examples of the provided sequences, a target range for a measurement can be characterized as range of values around a target value. For example, for a measurement of temperature, the target value can be a temperature capable of inducing cell death. For example, for a measurement of temperature, the target value can be a value that is greater than or equal to 42° C. For example, for a measurement of temperature, the target value can be 50° C. For example, for a measurement of temperature, the target value can be 55° C. For example, for a measurement of temperature, the target value can be 60° C. For example, for a measurement of temperature, the target value can be 65° C. For example, for a measurement of temperature, the target value can be 70° C. For example, for a measurement of temperature, the target value can be 75° C. For example, for a measurement of temperature, the target value can be 80° C. For example, for a measurement of temperature, the target value can be 85° C. For example, if a measurement is a temperature, the target value can be 90° C. For example, for a measurement of temperature, the target value can be 95° C. For example, for a measurement of temperature, the target value can be 100° C. For example, for a measurement of temperature, the target range can be +/−2° C. of a target temperature. For example, for a measurement of temperature, the target range can be +/−5° C. of a target temperature. For example, for a measurement of temperature, the target range can be +/−10° C. of a target temperature. For example, for a measurement of temperature, the target range can be +/−15° C. of a target temperature. For example, for a measurement of temperature, the target range can be +/−20° C. of a target temperature. For example, for a measurement of temperature, the target range can be +/−25° C. of a target temperature. For example, for a measurement of temperature, the target range can be +/−30° C. of a target temperature. For example, for a measurement of temperature, the target range can be +/−1° C. of a target temperature. For example, for a measurement of temperature, the target range can be a range around a target value, where the size of the range is configured to suit a clinical objective.

For example, for a measurement that is an impedance, the target value can be a value configured to indicate a temperature that is near 100° C. For example, for a measurement of impedance, the target value can be a value configured to indicate a temperature that is below 100° C. For example, for a measurement of impedance, the target value can be a value configured to indicate that tissue near an electrode is not boiling. For example, for a measurement of impedance, the target value can be 10 Ohms. For example, for a measurement of impedance, the target value can be 10 Ohms. For example, for a measurement of impedance, the target value can be 10 Ohms. For example, for a measurement of impedance, the target value can be 20 Ohms. For example, for a measurement of impedance, the target value can be 30 Ohms. For example, for a measurement of impedance, the target value can be 40 Ohms. For example, for a measurement of impedance, the target value can be 50 Ohms. For example, for a measurement of impedance, the target value can be 60 Ohms. For example, for a measurement of impedance, the target value can be 70 Ohms. For example, for a measurement of impedance, the target value can be 80 Ohms. For example, for a measurement of impedance, the target value can be 90 Ohms. For example, for a measurement of impedance, the target value can be 100 Ohms. For example, for a measurement of impedance, the target value can be 150 Ohms. For example, for a measurement of impedance, the target value can be 200 Ohms. For example, for a measurement of impedance, the target value can be 500 Ohms. For example, for a measurement of impedance, the target value can be 1000 Ohms. For example, for a measurement of impedance, the target value can be a value less than 10 Ohms. For example, for a measurement of impedance, the target value can be a value greater than 1000 Ohms. For example, for a measurement of impedance, the target range can be +/−10 Ohms of a target value. For example, for a measurement of impedance, the target range can be +/−20 Ohms of a target value. For example, for a measurement of impedance, the target range can be +/−30 Ohms of a target value. For example, for a measurement of impedance, the target range can be +/−50 Ohms of a target value. For example, for a measurement of impedance, the target range can be +/−100 Ohms of a target value. For example, for a measurement that is an impedance, the target range can contain values that are less than 10 Ohms of a target value. For example, for a measurement that is an impedance, the target range can contain values that are greater than 100 Ohms of a target value. For example, for a measurement that is an impedance, the target range can contain values that indicate the tissue near an electrodes is not boiling.

For example, for a measurement of power, the target power can be a value less than 50 Watts. For example, for a measurement of power, the target power can be a value greater than 50 Watts. For example, for a measurement of power, the target power can be 1 Watts. For example, for a measurement of power, the target power can be 5 Watts. For example, for a measurement of power, the target power can be 10 Watts. For example, for a measurement of power, the target power can be 20 Watts. For example, for a measurement of power, the target power can be 25 Watts. For example, for a measurement of power, the target power can be 50 Watts. For example, for a measurement of power, the target power can be 100 Watts. For example, for a measurement of power, the target power can be 200 Watts. For example, for a measurement of power, the target range can be +/−1 Watts of a target value. For example, for a measurement of power, the target range can be +/−2 Watts of a target value. For example, for a measurement of power, the target range can be +/−5 Watts of a target value. For example, for a measurement of power, the target range can be +/−10 Watts of a target value. For example, for a measurement of power, the target range can be +/−20 Watts of a target value. For example, for a measurement of power, the target range can contain values that are less than 1 W different from a target value. For example, for a measurement of power, the target range can contain values that are greater than 20 W different from a target value.

For example, for a measurement of voltage, the target voltage can be a value less than 100 Volts. For example, for a measurement of voltage, the target voltage can be a value greater than 100 Volts. For example, for a measurement of voltage, the target voltage can be 1 Volts. For example, for a measurement of voltage, the target voltage can be 5 Volts. For example, for a measurement of voltage, the target voltage can be 10 Volts. For example, for a measurement of voltage, the target voltage can be 15 Volts. For example, for a measurement of voltage, the target voltage can be 20 Volts. For example, for a measurement of voltage, the target voltage can be 25 Volts. For example, for a measurement of voltage, the target voltage can be 30 Volts. For example, for a measurement of voltage, the target voltage can be 50 Volts. For example, for a measurement of voltage, the target voltage can be 100 Volts. For example, for a measurement of voltage, the target voltage can be 200 Volts. For example, for a measurement of voltage, the target range can be +/−1 Volts of a target value. For example, for a measurement of voltage, the target range can be +/−2 Volts of a target value. For example, for a measurement of voltage, the target range can be +/−5 Volts of a target value. For example, for a measurement of voltage, the target range can be +/−10 Volts of a target value. For example, for a measurement of voltage, the target range can be +/−20 Volts of a target value. For example, for a measurement of voltage, the target range can be +/−25 Volts of a target value. For example, for a measurement of voltage, the target range can be +/−50 Volts of a target value. For example, for a measurement of voltage, the target range can contain values that are less than 1 V different from a target value. For example, for a measurement of voltage, the target range can contain values that are greater than 50 V different from a target value.

For example, for a measurement of current, the target current can be a value less than 1000 Milliamps. For example, for a measurement of current, the target current can be a value greater than 1000 Milliamps. For example, for a measurement of current, the target current can be 1 Milliamps. For example, for a measurement of current, the target current can be 5 Milliamps. For example, for a measurement of current, the target current can be 10 Milliamps. For example, for a measurement of current, the target current can be 50 Milliamps. For example, for a measurement of current, the target current can be 100 Milliamps. For example, for a measurement of current, the target current can be 200 Milliamps. For example, for a measurement of current, the target current can be 250 Milliamps. For example, for a measurement of current, the target current can be 500 Milliamps. For example, for a measurement of current, the target current can be 1000 Milliamps. For example, for a measurement of current, the target current can be 2000 Milliamps. For example, for a measurement of current, the target range can be +/−1 Milliamps of a target value. For example, for a measurement of current, the target range can be +/−2 Milliamps of a target value. For example, for a measurement of current, the target range can be +/−5 Milliamps of a target value. For example, for a measurement of current, the target range can be +/−10 Milliamps of a target value. For example, for a measurement of current, the target range can be +/−20 Milliamps of a target value. For example, for a measurement of current, the target range can be +/−25 Milliamps of a target value. For example, for a measurement of current, the target range can be +/−50 Milliamps of a target value. For example, for a measurement of current, the target range can be +/−100 Milliamps of a target value. For example, for a measurement of current, the target range can be +/−200 Milliamps of a target value. For example, for a measurement of current, the target range can be +/−500 Milliamps of a target value. For example, for a measurement of current, the target range can contain values that are less than 1 Milliamps different from a target value. For example, for a measurement of current, the target range can contain values that are greater than 2000 mA different from a target value.

For example, for a measure of any type, the target value can be suited to a clinical objective. For example, for a measure of any type, the range of target values can be suited to a clinical objective. For example, for a measure of any type, the size of the target range can be different for different target values. For example, for a measure of any type, the target range can be specified relative to the target value. For example, for a measure of any type, the target range can be a specified as a percentage of the target value. For example, for a measurement of any type, the target range can be +/−1% of a target value. For example, for a measurement of any type, the target range can be +/−2% of a target value. For example, for a measurement of any type, the target range can be +/−5% of a target value. For example, for a measurement of any type, the target range can be +/−10% of a target value. For example, for a measurement of any type, the target range can be +/−15% of a target value. For example, for a measurement of any type, the target range can be +/−20% of a target value. For example, for a measurement of any type, the target range can be +/−25% of a target value. For example, for a measurement of any type, the target range can be +/−30% of a target value. For example, for a measurement of any type, the target range can be greater than +/−30% of a target value.

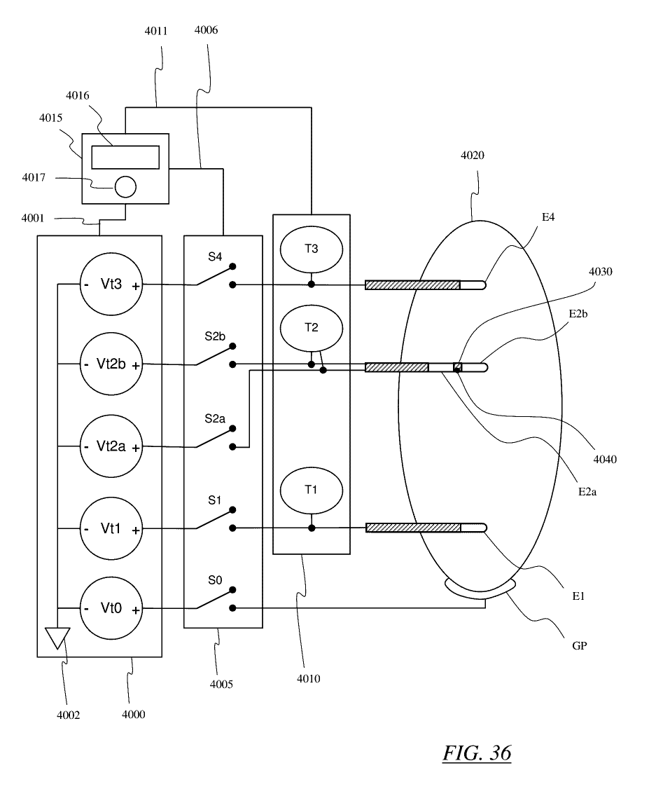

Referring now to FIG. 36 and in accordance with one example of the present invention, an example of a system configured for delivery of electrical output to a living body 4020 is presented. In this example, the power supply unit 4000 is configured to produce more than two poles of electrical output to which electrodes E1, E2a, E2b, E3 and reference ground pad GP can be connected via the switching system 4005. Electrodes E2a and E2b are mechanically connected but electrically isolated by an electrically-insulating element 4030 at their interface. In one example, Electrode E2a, electrode E2b, and their relative positions can be configured so that the region of influence of both electrodes has substantial common tissue volume. For example, electrodes E2a and E2b can be sized and positioned such that a temperature measured at location 4040 is indicative of the temperature at both E2a and E2b. In one example, electrodes E2a and E2b each have substantially the same effect on the tissue 4020. Each electrode E1, E2a, E2b, E3 can be connected and disconnected from the power supply 4000 by closing and opening switches S1, S2a, S2b, S3, respectively. The reference ground pad GP can be connected and disconnected from the power supply 4000 by closing and opening switch S0. The measurement system 4010 can collect measurements T1, T2, T3, where T1 is associated with electrode E1, T2 is associated with both electrodes E2a and E2b, and T3 is associated with electrode 3. In one example, measurements T1, T2, T3 can include a temperature. In one example, a temperature sensor 4040 can be configured to indicate the common temperature of both electrodes E2a and E2b and that temperature can be measured by T2. For example, the temperature sensor 4040 can be situated at the interface of the two electrodes E2a and E2b. In one example, measurements T1, T2, T3 can include a current. In one example, T2 can measure the total current delivered to both electrodes E2a and E2b. In one example, measurements T1, T2, T3 can include a power. In one example, T2 can measure the total power delivered to both electrodes E2a and E2b. In one example, measurements T1, T2, T3 can include a voltage. In one example, T2 can measure the voltage delivered to both electrodes E2a and E2b. In one example, measurements T1, T2, T3 can include the average current over more than one step in a sequence of switch states. In one example, measurements T1, T2, T3 can include the average power over more than one step in a sequence of switch states. In one example, measurements T1, T2, T3 can include the average voltage over more than one step in a sequence of switch states. In one example, measurements T1, T2, T3 can include an impedance. In one example, T2 can measure an aggregate measurement derived from the impedances of both electrodes E2a and E2b, such as the average of the impedance from E2a and the impedance from E2b, relative to some reference potential.

The controller 4015 is connected to the power supply 4000, switches 4005, and measurement system 4010. The controller can coordinate the actions of the power supply 4000, switches 4005, and measurement system 4010. For example, the controller can implement feedback control of the power supply 4000 and switches 4005 based on measurements T1, T2, T3 from the measurement system 4015.

Power supply 4000 consists of voltage supplies Vt0, Vt1, Vt2a, Vt2b, Vt3, referenced to a common reference potential 4002. The controller 4015 can control each of the voltage supplies independently. In one example, each voltage supply can produce a different output signal. In one example, the voltage supplies Vt0, Vt1, Vt2a, Vt2b, Vt3 can produce radiofrequency signals. In one example, a two-pole system can be produced by setting voltage supplies Vt0, Vt1, Vt2a, Vt2b, Vt3 such that each supply produces one of two output signals. For example, in one specific example of a two-pole system, a sequence of power supply settings can include a step in which Vt0=Vt3=V+ and Vt1=Vt2a=Vt2b=V−, and another step in which Vt1=Vt3=V+ and Vt0=Vt2a=Vt2b=V−, such that V+ and V− can be set individually by the controller. In one example, a three-pole system can be produced by setting voltage supplies Vt0, Vt1, Vt2a, Vt2b, Vt3 such that each supply produces one of three output signals. For example, in one specific example of a three-pole system, a sequence of power supply settings can include a step in which Vt0=V+, Vt1=V−, and Vt2a=Vt2b=Vt3=V*; and a step in which Vt1=V+, Vt2a=Vt2b=V−, and Vt3=Vt0=V*; where V+, V−, and V* can be set individually by the controller. In one example, a four-pole system can be produced by assigning output signals to each of Vt0, Vt1, Vt2a=Vt2b, Vt3.

In one example, it is understood that the system presented in FIG. 36 can be operated in the manner presented in FIGS. 29, 30, 31, 32, 33, and 35 by splitting the output delivered to electrode “E2” in FIGS. 29, 30, 31, 32, 33, and 35, apportioning part of the output to electrode E2a (of FIG. 36) and apportioning the remaining part of the output to electrode E2b (of FIG. 36). For example, for each step of the sequences in FIGS. 29, 30, 31, 32, 33, and 35 where “E2” is connected to an output pole of the generator, instead E2a can be connected to that output for half the duration of the step, and E2b can be connected to that output pole for the remaining duration of the step.

In another example, the system in FIG. 36 can be expanded to accommodate and additional electrode E4 associated with measurement T4, and the augmented system can be operated in the manner presented in FIG. 34 by splitting the output delivered to electrode “E2” in FIGS. 29, 30, 31, 32, 33, and 35, apportioning part of the output to electrode E2a (of FIG. 36) and apportioning the remaining part of the output to electrode E2b (of FIG. 36). In another example, it is understood that the system in FIG. 36 can be expanded to accommodate any number of additional electrodes.

It is understood that, in another example, more than two electrodes can be configured as are electrodes E2a and E2b shown in FIG. 36, such that the said more than two electrodes are positioned and sized such that a single measurement can characterize a parameter that is common to all said more than two electrodes, such as the parameter of temperature. For example, three electrodes can be positioned around a central temperature sensor. For example, for the six-electrode device presented in FIG. 21, the electrodes 961, 962, 963, 964, 965, 966 can be sized and spaced such that a temperature probe can be placed between each pair of electrodes in order that the temperature distribution along the length of the probe can be controlled.

In another aspect, the examples of FIGS. 1 through 36 can also include bipolar multiplexing and/or switching between groups of electrodes that each have internal cooling channels and the high frequency system can include a source of coolant fluid that can be circulated through the electrodes in a way similar to the systems and electrodes shown in the references herein. Therefore the multiplexed bipolar system and method claimed in this patent can also include cooled high frequency electrodes, coolant supplies, and related control of the coolant rate and temperature in the production of the high frequency lesioning.

A number of embodiments of the invention have been described. Nevertheless, it will be understood that various modifications may be made without departing from the spirit and scope of the invention. Accordingly, other embodiments are within the scope of the following claims:

1. A system for the application of electrical energy to bodily tissue comprising:
At least three electrodes, each of the at least three electrodes having an exposed conductive tip and a temperature sensor in the conductive tip, each electrode being adapted to be inserted into a patient's body so that said conductive tip will contact the tissue in the patient's body and the temperature sensor will sense the temperature of the tissue near said conductive tip;
a generator that produces high-frequency signal output between at least two output poles;
a temperature detector that measures the temperatures from the temperature sensors;
a switching system by which each of the at least three electrodes can be connected to and disconnected from each of the at least two output poles; and
a controller that automatically produces a sequence of steps to regulate at the same time the temperatures measured from all of the at least three electrodes, such that the temperature measured from each of the at least three electrodes is held at a set temperature for the electrode; wherein, for each of the steps of the sequence, the controller configures the switching system to connect one or more of the at least three electrodes to a first output pole of the least two output poles forming a first group of electrodes, and to connect one or more of the at least three electrodes to a second output pole of the at least two output poles forming a second group of electrodes, so that high-frequency signal output is electrically conducted through the patient's body between the first group of electrodes and the second group of electrodes; the controller sets the duration of each of the steps in the sequence, the identity of electrodes in the first group for each of the steps in the sequence, and the identity of the electrodes in the second group for each of the steps in the sequence; and during the sequence, the generator does not deliver high-frequency signal output to any electrode in contact with the patient's body other than the at least three electrodes.

2. (canceled)

3. The system of claim 1 wherein the high frequency signal output of said generator is in the radiofrequency frequency range.

4. The system of claim 1 wherein the said at least three electrodes include cooled electrodes.

5. A method for the application of electrical energy to bodily tissue comprising:
Inserting at least three electrodes into a patient's body, the at least three electrodes each having an exposed conductive tip and a temperature sensor in the conductive tip so that said conductive tip will contact the tissue in the patient's body and the temperature sensor will sense the temperature of the tissue near said conductive tip;
connecting the said at least three electrodes through a controller to a generator that produces a high frequency signal output across a first output jack and a second output jack;
connecting the at least three electrodes to a temperature detector a temperature detector that measures the temperatures from the temperature sensors;
connecting the at least three electrodes to a controller that can connect to the first and the second output jacks and can connect to the at least three electrodes, the controller comprising a switching system that enables switching the said signal output from said first jack to a subset of n electrodes of said least three electrodes and switching the signal output from the said second jack to a different subset of m electrodes of said at least three electrodes;
said controller comprising a control algorithm that controls the signal output, the switching sequences of the switching system, the choice of the said n electrodes and the said m electrodes in each step of the switching sequences, and the duration of the connection of said signal output in each step of the switching sequences, so that the temperatures detected by said temperature detector achieves a temperature distribution objective at said at least three electrodes; and
supplying said signal output through said controller while measuring the temperatures on the said at least three electrodes, and initiating said control algorithm to bring said measured temperatures at said temperature sensors to the temperature distribution objective.

6. The method of claim 2 comprising inserting said at least three electrodes into the region of innervations of the sacroiliac joint in a patient's body and initiating the lesioning to achieve a temperature distribution objective to reduce pain in the SI joint.

7. The method of claim 2 comprising inserting said at least three electrodes into the region of innervations of the spine in the patient's body and initiating the lesioning for to achieve a temperature distribution objective reduce pain in the spine.

8. The method of claim 2 comprising inserting said at least three electrodes into the region of a tumor in the patient's body and initiating the lesioning for to achieve a temperature distribution objective reduce thermally ablate the tumor.

9. A system for the application of electrical energy to bodily tissue comprising:
At least two electrodes each having an exposed conductive tip and a temperature sensor in the conductive tip, the at least two electrode being adapted to be inserted into a patient's body so that said conductive tip will contact the tissue in the patient's body and the temperature sensor will sense the temperature of the tissue near said conductive tip;
a reference electrode that is adapted to be placed on the skin of the patient's body;
a generator that produces a high frequency signal output across a first output jack and a second output jack;
a temperature detector that measures the temperatures from the temperature sensors; and
a controller that can connect to the first and the second output jacks and can connect to said at least two electrodes and to said reference electrode, the controller comprising a switching system that enables switching the said signal output from said first jack to a subset of n electrodes of said least two electrodes and switching the signal output from the said second jack to a different subset of m electrodes of said at least two electrodes and to the said reference electrode;
said controller comprising a control algorithm that controls the signal output; the switching sequences of the switching system; the choice of the said n electrodes, the said m electrodes, and the said reference electrode in each step of the switching sequences; and the duration of the connection of said signal output in each step of the switching sequences, so that the temperatures detected by said temperature detector achieve a temperature distribution objective on the said at least two electrodes.

10. The system of claim 9 wherein the temperature distribution objective includes having the temperatures on said at least three electrodes rise to a set temperature level.

11. The system of claim 9 wherein the high frequency signal output of said generator is in the radiofrequency frequency range.

12. The system of claim 9 wherein the said at least three electrodes include cooled electrodes.

13. A method for the application of electrical energy to bodily tissue comprising:
Inserting at least two electrodes into a patient's body, the said at least two electrodes each having an exposed conductive tip and a temperature sensor in the conductive tip so that said conductive tip will contact the tissue in the patient's body and the temperature sensor will sense the temperature of the tissue near said conductive tip;
placing a reference electrode on the skin of the patient's body;
connecting the said at least two electrodes and the said reference electrode through a controller to a generator that produces a high frequency signal output across a first output jack and a second output jack;
connecting the said at least two electrodes to a temperature detector that measures the temperatures from the temperature sensors;
connecting the said at least two electrodes and the said reference electrode to a controller that can connect to the first and the second output jacks, to the said at least two electrodes, and to the said reference electrode;
said controller comprising a switching system that enables switching the said signal output from said first jack to a subset of n electrodes of said least two electrodes and switching the signal output from the said second jack to a different subset of m electrodes of said at least two electrodes and to the said reference electrode;
said controller comprising a control algorithm that controls the signal output; the switching sequences of the switching system; the choice of the said n electrodes, the said m electrodes, and the said reference electrode in each step of the switching sequences; and the duration of the connection of said signal output in each step of the switching sequences, so that the temperatures detected by said temperature detector achieve a temperature distribution objective on the said at least two electrodes; and
supplying said signal output through said controller while measuring the temperatures on the said at least two electrodes and initiating said control algorithm to bring said measured temperatures at said temperature sensors to the temperature distribution objective.

14. The method of claim 13 comprising inserting said at least three electrodes into the region of innervations of the sacroiliac joint in a patient's body and initiating the lesioning to achieve a temperature distribution objective to reduce pain in the SI joint.

15. The method of claim 13 comprising inserting said at least three electrodes into the region of innervations of the spine in the patient's body and initiating the lesioning for to achieve a temperature distribution objective reduce pain in the spine.

16. The method of claim 13 comprising inserting said at least three electrodes into the region of a tumor in the patient's body and initiating the lesioning for to achieve a temperature distribution objective reduce thermally ablate the tumor.

17. A system for the application of electrical energy to bodily tissue comprising:
At least three electrodes, each of the at least three electrodes having an exposed conductive tip and a temperature sensor in the conductive tip, each electrode being adapted to be inserted into a patient's body so that said conductive tip will contact the tissue in the patient's body and the temperature sensor will sense the temperature of the tissue near said conductive tip;
a generator that produces high-frequency signal output between at least two output poles;
a switching system by which each of the at least three electrodes can be connected to and disconnected from each of the output poles; and
a controller that automatically produces a sequence of at least two steps; wherein, for each step in the sequence, the controller configures the switching system to connect one or more of the at least three electrodes to the first output pole forming a first group of electrodes and to connect one or more of the at least three electrodes to the second output pole forming a second group of electrodes, and the controller determines the duration of each step in the sequence, an identity of the electrodes in the first group for each step in the sequence, and the identity of the electrodes in the second group for each step in the sequence; such that for at least one step in the sequence, a total number of the at least three electrodes that are in the union of the first group and the second group for the step is greater than two; and such that the first group during a first step in the sequence is different from the first group during a second step in the sequence, and the second group during the first step is different from the second group during the second step.

18. A system for the application of electrical energy to bodily tissue comprising:
at least three electrodes each comprising a conductive element adapted to be placed in contact with bodily tissue;
a generator that produces an electrical signal output across a first output jack and a second output jack;
a controller that can connect to the said first and second output jacks and can connect to said at least three electrodes;
the said controller comprising a switching system that enables switching the said signal output from said first jack to a subset of n electrodes of the said least three electrodes and switching the signal output from the said second jack to a different subset of m electrodes of the said at least three electrodes;
the said controller comprising a measurement system that measures a parameter for each of the said at least three electrodes;
the said controller comprising a control algorithm that controls the signal output, the switching sequences of the switching system, the choice of the said n electrodes and the said m electrodes in each step of the switching sequences, and the duration of the connection of said signal output in each step of the switching sequences so that the values of all said measured parameters can be brought within their respective targeted ranges.

19. The system in claim 18, where one parameter is the average power delivered to an electrode over a duration that exceeds one step of a switching sequence.

20. The system in claim 18, where one parameter is root-mean-squared current delivered to an electrode over a duration that exceeds one step of a switching sequence.

21. The system in claim 18, where one parameter is root-mean-squared voltage delivered to an electrode over a duration that exceeds one step of a switching sequence.

22. The system in claim 18, where one parameter is a function of the electrical signal delivered to an electrode over a duration that exceeds one step of a switching sequence.

23. The system in claim 18, where one parameter is a function of the electrical signal delivered to an electrode over a duration that is less than or equal to one step of a switching sequence.

24. The system in claim 18, where one parameter is the electrical impedance between an electrode and another structure or structures.

25. The system in claim 18, where one parameter is the electrical resistance between an electrode and another structure or structures.

26. The system in claim 18, where one parameter is the temperature of an electrode.

27. A system for the application of electrical energy to bodily tissue comprising:
at least three electrodes each comprising a conductive element adapted to be placed in contact with bodily tissue;
a generator that produces an electrical signal output across a first output jack and a second output jack;
a controller that can connect to the said first and second output jacks and can connect to said at least three electrodes;
the said controller comprising a switching system that enables switching the said signal output from said first jack to a subset of n electrodes of the said least three electrodes and switching the signal output from the said second jack to a different subset of m electrodes of the said at least three electrodes;
the said controller comprising a measurement system that measures parameters whose number exceeds that of the number of electrodes;
the said controller comprising a control algorithm that controls the signal output, the switching sequences of the switching system, the choice of the said n electrodes and the said m electrodes in each step of the switching sequences, and the duration of the connection of said signal output in each step of the switching sequences so that the values of all said measured parameters can be brought within their respective targeted ranges.

28. The system in claim 27, where one parameter is the average power delivered between two electrodes over a duration that exceeds one step of a switching sequence.

29. The system in claim 27, where one parameter is root-mean-squared current delivered between two electrodes over a duration that exceeds one step of a switching sequence.

30. The system in claim 27, where one parameter is root-mean-squared voltage delivered between two electrodes over a duration that exceeds one step of a switching sequence.

31. The system in claim 27, where one parameter is a function of the electrical signal delivered between two electrodes over a duration that exceeds one step of a switching sequence.

32. The system in claim 27, where one parameter is a function of the electrical signal delivered between two electrodes over a duration that is less than or equal to one step of a switching sequence.

33. The system in claim 27, where one parameter is the electrical impedance between two electrodes.

34. The system in claim 27, where one parameter is the electrical resistance between two electrodes.

35. The system in claim 27, where one parameter is the temperature measured between two electrodes.

36. A system for the application of electrical energy to bodily tissue comprising:
At least two electrodes each having an exposed conductive tip and a temperature sensor in the conductive tip, the at least two electrode being adapted to be inserted into a patient's body so that said conductive tip will contact the tissue in the patient's body and the temperature sensor will sense the temperature of the tissue near said conductive tip;
a reference electrode that is adapted to be placed on the skin of the patient's body;
a generator that produces a high frequency signal output across a first output jack and a second output jack;
a temperature detector that measures the temperatures from the temperature sensors; and
a controller that can connect to the first and the second output jacks and can connect to said at least two electrodes and to said reference electrode, the controller comprising a switching system that enables switching the said signal output from said first jack to a subset of n electrodes of said least two electrodes and switching the signal output from the said second jack to a different subset of m electrodes of said at least two electrodes and to the said reference electrode;
the said controller comprising a measurement system that measures a parameter for each of the said at least two electrodes;
the said controller comprising a control algorithm that controls the signal output, the switching sequences of the switching system, the choice of the said n electrodes and the said m electrodes in each step of the switching sequences, and the duration of the connection of said signal output in each step of the switching sequences so that the values of all said measured parameters can be brought within their respective targeted ranges.

37. The system in claim 36, where one parameter is the average power delivered to an electrode over a duration that exceeds one step of a switching sequence.

38. The system in claim 36, where one parameter is root-mean-squared current delivered to an electrode over a duration that exceeds one step of a switching sequence.

39. The system in claim 36, where one parameter is root-mean-squared voltage delivered to an electrode over a duration that exceeds one step of a switching sequence.

40. The system in claim 36, where one parameter is a function of the electrical signal delivered to an electrode over a duration that exceeds one step of a switching sequence.

41. The system in claim 36, where one parameter is a function of the electrical signal delivered to an electrode over a duration that is less than or equal to one step of a switching sequence.

42. The system in claim 36, where one parameter is the electrical impedance between an electrode and another structure or structures.

43. The system in claim 36, where one parameter is the electrical resistance between an electrode and another structure or structures.

44. The system in claim 36, where one parameter is the temperature of an electrode.

45. A system for the application of electrical energy to bodily tissue comprising:
At least two electrodes each having an exposed conductive tip and a temperature sensor in the conductive tip, the at least two electrode being adapted to be inserted into a patient's body so that said conductive tip will contact the tissue in the patient's body and the temperature sensor will sense the temperature of the tissue near said conductive tip;
a reference electrode that is adapted to be placed on the skin of the patient's body;
a generator that produces a high frequency signal output across a first output jack and a second output jack;
a temperature detector that measures the temperatures from the temperature sensors; and
a controller that can connect to the first and the second output jacks and can connect to said at least two electrodes and to said reference electrode, the controller comprising a switching system that enables switching the said signal output from said first jack to a subset of n electrodes of said least two electrodes and switching the signal output from the said second jack to a different subset of m electrodes of said at least two electrodes and to the said reference electrode;
the said controller comprising a measurement system that measures parameters whose number exceeds that of the number of electrodes;
the said controller comprising a control algorithm that controls the signal output, the switching sequences of the switching system, the choice of the said n electrodes and the said m electrodes in each step of the switching sequences, and the duration of the connection of said signal output in each step of the switching sequences so that the values of all said measured parameters can be brought within their respective targeted ranges.

46. The system in claim 45, where one parameter is the average power delivered between two electrodes over a duration that exceeds one step of a switching sequence.

47. The system in claim 45, where one parameter is root-mean-squared current delivered between two electrodes over a duration that exceeds one step of a switching sequence.

48. The system in claim 45, where one parameter is root-mean-squared voltage delivered between two electrodes over a duration that exceeds one step of a switching sequence.

49. The system in claim 45, where one parameter is a function of the electrical signal delivered between two electrodes over a duration that exceeds one step of a switching sequence.

50. The system in claim 45, where one parameter is a function of the electrical signal delivered between two electrodes over a duration that is less than or equal to one step of a switching sequence.

51. The system in claim 45, where one parameter is the electrical impedance between two electrodes.

52. The system in claim 45, where one parameter is the electrical resistance between two electrodes.

53. The system in claim 45, where one parameter is the temperature measured between two electrodes.

54. A system consisting of at least two electrical output poles that can generate different electrical potentials, of at least three electrodes that are configured to deliver electrical output to a living body, and of a measurement system that can measure a parameter associated with each of said electrodes; where said system is configured to generate a sequence of connections between said electrodes and said electrical output poles; where, during each step of said sequence, said system can control the signal output delivered to said output poles, the connections between output poles and electrodes, and the duration of the step, for the purpose of controlling all said measured parameters at the same time.

55. The system in claim 54, where one potential is a high frequency potential.

56. The system in claim 54, where one parameter is the average power delivered to an electrode over a duration that exceeds one step of a switching sequence.

57. The system in claim 54, where one parameter is root-mean-squared current delivered to an electrode over a duration that exceeds one step of a switching sequence.

58. The system in claim 54, where one parameter is root-mean-squared voltage delivered to an electrode over a duration that exceeds one step of a switching sequence.

59. The system in claim 54, where one parameter is a function of the electrical signal delivered to an electrode over a duration that exceeds one step of a switching sequence.

60. The system in claim 54, where one parameter is a function of the electrical signal delivered to an electrode over a duration that is less than or equal to one step of a switching sequence.

61. The system in claim 54, where one parameter is the electrical impedance between an electrode and another structure or structures.

62. The system in claim 54, where one parameter is the electrical resistance between an electrode and another structure or structures.

63. The system in claim 54, where one parameter is the temperature of an electrode.

64. A system consisting of at least two electrical output poles that can generate different electrical potentials, of at least two treatment electrodes that are configured to deliver electrical output to a living body, of at least reference electrode, and of a measurement system that can measure a parameter associated with each of said treatment electrodes; where said system is configured to generate a sequence of system states; where, during each step of said sequence, said system can control the signal output delivered to said output poles, the treatment electrodes and reference electrodes that are connected to each output pole, and the duration of the step, for the purpose of controlling all said measured parameters at the same time.

65. The system in claim 64, where one potential is a high frequency potential.

66. The system in claim 64, where one reference electrode is a ground pad configured to be place on a skin said living body.

67. The system in claim 64, where one parameter is the average power delivered to a treatment electrode over a duration that exceeds one step of a switching sequence.

68. The system in claim 64, where one parameter is root-mean-squared current delivered to a treatment electrode over a duration that exceeds one step of a switching sequence.

69. The system in claim 64, where one parameter is root-mean-squared voltage delivered to a treatment electrode over a duration that exceeds one step of a switching sequence.

70. The system in claim 64, where one parameter is a function of the electrical signal delivered to a treatment electrode over a duration that exceeds one step of a switching sequence.

71. The system in claim 64, where one parameter is a function of the electrical signal delivered to a treatment electrode over a duration that is less than or equal to one step of a switching sequence.

72. The system in claim 64, where one parameter is the electrical impedance between a treatment electrode and another structure or structures.

73. The system in claim 64, where one parameter is the electrical resistance between a treatment electrode and another structure or structures.

74. The system in claim 64, where one parameter is the temperature of a treatment electrode.

75. A system consisting of at least two electrical output poles that can generate different electrical potentials, of at least three electrodes that are configured to deliver electrical output to a living body, and of a measurement system that can measure more parameters than the number of electrodes; where said system is configured to generate a sequence of connections between said electrodes and said electrical output poles; where, during each step of said sequence, said system can control the signal output delivered to said output poles, the connections between output poles and electrodes, and the duration of the step, for the purpose of controlling all said measured parameters at the same time.

76. The system in claim 75, where one potential is a high frequency potential.

77. The system in claim 75, where one parameter is the average power delivered between two electrodes over a duration that exceeds one step of a switching sequence.

78. The system in claim 75, where one parameter is root-mean-squared current delivered between two electrodes over a duration that exceeds one step of a switching sequence.

79. The system in claim 75, where one parameter is root-mean-squared voltage delivered between two electrodes over a duration that exceeds one step of a switching sequence.

80. The system in claim 75, where one parameter is a function of the electrical signal delivered between two electrodes over a duration that exceeds one step of a switching sequence.

81. The system in claim 75, where one parameter is a function of the electrical signal delivered between two electrodes over a duration that is less than or equal to one step of a switching sequence.

82. The system in claim 75, where one parameter is the electrical impedance between two electrodes.

83. The system in claim 75, where one parameter is the electrical resistance between two electrodes.

84. The system in claim 75, where one parameter is the temperature measured between two electrodes.

85. A system consisting of at least two electrical output poles that can generate different electrical potentials, of at least two treatment electrodes that are configured to deliver electrical output to a living body, of at least reference electrode, and of a measurement system that can measure can measure more parameters than the number of treatment electrodes; where said system is configured to generate a sequence of system states; where, during each step of said sequence, said system can control the signal output delivered to said output poles, the treatment electrodes and reference electrodes that are connected to each output pole, and the duration of the step, for the purpose of controlling all said measured parameters at the same time.

86. The system in claim 85, where one potential is a high frequency potential.

87. The system in claim 85, where one reference electrode is a ground pad configured to be place on a skin said living body.

88. The system in claim 85, where one parameter is the average power delivered between two treatment electrodes over a duration that exceeds one step of a switching sequence.

89. The system in claim 85, where one parameter is root-mean-squared current delivered between two treatment electrodes over a duration that exceeds one step of a switching sequence.

90. The system in claim 85, where one parameter is root-mean-squared voltage delivered between two treatment electrodes over a duration that exceeds one step of a switching sequence.

91. The system in claim 85, where one parameter is a function of the electrical signal delivered between two treatment electrodes over a duration that exceeds one step of a switching sequence.

92. The system in claim 85, where one parameter is a function of the electrical signal delivered between two treatment electrodes over a duration that is less than or equal to one step of a switching sequence.

93. The system in claim 85, where one parameter is the electrical impedance between two treatment electrodes.

94. The system in claim 85, where one parameter is the electrical resistance between two treatment electrodes.

95. The system in claim 85, where one parameter is the temperature measured between two treatment electrodes.

96. A system consisting of at least two electrical output poles that can generate different electrical potentials, and of at least three electrodes that are configured to deliver electrical output to a living body; where said system is configured to generate a sequence of connections between said electrodes and said electrical output poles; where at least two steps in said sequence differ in the connections made between said electrodes and said electrical output poles; where said sequence contains at least one step is which each of at least three electrodes is connected to an electrical output pole; and where no electrode serves as the path for return currents from other electrodes in all steps of said sequence.

97. The system in claim 96 where said sequence can be generated automatically.

98. A system consisting of at least two electrical output poles that can generate different electrical potentials, and of at least three electrodes that are placed in a living body;
where no electrode is a ground pad; where said system is configured to generate a sequence of connections between said electrodes and said electrical output poles; where at least two steps in said sequence differ in the connections made between said electrodes and said electrical output poles; where said sequence contains at least one step is which each of at least three electrodes is connected to an electrical output pole.

99. The system in claim 98 where said sequence can be generated automatically.

100. The system of claim 1 wherein the generator produces high-frequency signal output across exactly two output poles, each output pole having a different electrical potential from that of the other output pole.

101. The system of claim 1, further comprising a reference electrode that is not one of the at least three electrodes, wherein during the sequence, the reference electrodes is disconnected from the generator so that high-frequency signal output is not substantially conducted from the reference electrode into the patient's body.

102. The system of claim 101 wherein the reference electrode is adapted to contact the patient's skin or other anatomy remote of a treatment location.

103. The system of claim 101 wherein the reference electrode is adapted to penetrate into the patient's body and includes a temperature sensor.

104. The system of claim 1 wherein for each of the steps in the sequence, the first group of electrodes includes one and only one of the at least three electrodes, and the second group of electrodes includes one and only one other electrode of the at least three electrodes.

105. The system of claim 1 wherein for each of the steps in the sequence, the first group of electrodes includes one and only one of the at least three electrodes, and the second group of electrodes includes at least two other electrodes of the at least three electrodes.

106. The system of claim 1 wherein for each of the steps in the sequence, the first group of electrodes includes one and only one of the at least three electrodes, and the second group of electrodes comprises all of the other at least three electrodes.

107. The system of claim 1 wherein the temperature measured from each of the at least three electrodes is held within 2 degrees Centigrade of the set temperature for the electrode.

108. The system of claim 1 wherein the set temperatures are identical for all of the at least three electrodes.

109. The system of claim 1 wherein each set temperature is a value selected from the range 45 to 95 degrees Centigrade.

110. The system of claim 1 wherein each set temperature is a value selected from the range 37 to 42 degrees Centigrade.

111. The system of claim 1 wherein the set temperature for at least one of the at least three electrodes is configured to produced thermal damage to tissue in the patient's body.

112. The system of claim 1 wherein the set temperature for at least one of the at least three electrodes is configured to prevent substantial thermal damage to nerve cells within the patient's body.

113. The system of claim 1 wherein each of the at least three electrodes is configured for independent percutaneous placement of its conductive tip near a medial branch nerve within the patient's body.

114. The system of claim 1 wherein each of the at least three electrodes is configured for independent percutaneous placement of its conductive tip near the dorsal innervation of a sacroiliac joint within the patient's body, and the sequence is configured to preferentially heat the spaces in between adjacent pairs of the at least three electrodes.

115. A system for delivering electrical energy to a bodily tissue by conducting radiofrequency current through the bodily tissue among, and only among, at least three treatment electrodes in contact with the bodily tissue, wherein the system measures a temperature for each of the treatment electrodes, and the system raises and regulates the temperatures measured for all of the treatment electrodes at the same time such that the temperature measured at each of the treatment electrodes is held at a set temperature value for that electrode.
